(19) United States
(12) Patent Application Publication
(10) Pub. No.: US 2005/0019927 A1
(43) Pub. Date: Jan. 27, 2005

(54) DECREASING GENE EXPRESSION IN A MAMMALIAN SUBJECT IN VIVO VIA AAV-MEDIATED RNAi EXPRESSION CASSETTE TRANSFER

(76) Inventors: Markus Hildinger, Boston, MA (US); Alberto Auricchio, Napoli (IT)

Correspondence Address: MARKUS HILDINGER, ONE DEVONSHIRE PLACE, APT. 3401, BOSTON, MA, 02109, US

(21) Appl. No.: 10/604,340

(22) Filed: Jul. 13, 2003

(51) Int. Cl. A61K 48/00; C12N 15/86; C12N 5/02

(52) U.S. Cl. 435/456; 435/375; 514/44

(57) ABSTRACT

Decreasing the expression of genes in a mammalian subject has multiple applications ranging from cancer therapy to anti-infective therapy or treatment of autosomal dominant genetic disorders. Yet, there is still a lack of efficient technologies to achieve that goal in mammalian subjects in vivo. The present invention relates to methods for decreasing gene expression by administering to a mammalian subject a recombinant adeno-associated viral vector in vivo with said vector comprising an RNA interference (RNAi) expression cassette whose RNA expression products directly or indirectly lead to a decrease in expression of the corresponding RNAi target gene. Upon successful transduction with the recombinant adeno-associated viral vector, the RNA expression products of the RNAi expression cassette will decrease the cellular concentration of the mRNA transcripts of the RNAi target gene, thus resulting in decreased concentration of the protein encoded by the RNAi target gene.

### BACKGROUND OF INVENTION

(1) Field of the Invention

The present invention relates generally to methods for altering gene expression in a cell of a mammalian subject using recombinant adeno-associated viral vectors engineered to express one or more RNA molecules that induce RNA interference in said cell. In a more specific aspect, gene expression is decreased or down-regulated by administering in vivo to a mammalian subject a recombinant adeno-associated viral vector with said vector comprising an RNA interference (RNAi) expression cassette whose RNA expression product(s) directly or indirectly lead to a decrease in expression of the corresponding RNAi target gene.

Upon successful transduction with the recombinant adeno-associated viral vector, the RNA expression products of the RNAi expression cassette will decrease the cellular concentration of the mRNA transcript of the RNAi target gene, thus resulting in decreased concentration of the protein encoded by the RNAi target gene.

(2) Background of the Invention

(2.1) General Usefulness of Decreasing Expression of a Specific Gene In Vivo

Decreasing expression of a specific gene in a mammalian subject has multiple utilities in the medical field such as:
(1) Treatment of diseases where an endogenous gene is pathologically overexpressed, e.g., Tumor Necrosis Factor alpha in Rheumatoid Arthritis
(2) Treatment of genetically inherited diseases where one or more alleles are mutated, and the mutated allele(s) have pathologic effects, e.g., mutations in the Rhodopsin gene in autosomal-dominant Retinitis Pigmentosa
(3) Treatment of cancer where overexpression of a gene results into cancer, e.g., overexpression of Ras or the Epidermal Growth Factor Receptor (EGFR).
(4) Treatment of infectious diseases, primarily viral diseases, eases, where (a) exogenous (e.g., viral) genes contribute to disease pathogenesis (e.g., viral spread), such as the HIV integrase gene in AIDS; (b) endogenous genes contribute to disease pathogenesis (e.g., viral spread), such as the CCR5 Receptor gene in AIDS.

Thus, a method would be desirable that results in down-regulation of the expression of a specific gene with (1) high versatility/flexibility in terms of genes that can be targeted (i.e., broad potential applications); (2) high specificity for the target gene (i.e., no inadvertent inhibition of other genes); (3) high efficacy in terms of expression down-regulation of the target gene; (4) low/no side effects.

(2.2) Limitations of Prior Approaches

Different strategies have been tried so far to decrease gene expression such as
(1) nucleic acid based strategies, such as (a) ribozymes [1]; (b) antisense oligonucleotides [2]; and
(2) protein-based approaches, such as (a) artificial transcription factors [3, 4]; (b) intrabodies [5]

Unfortunately, their utility is limited mainly due to several factors:
(1) Their efficacy varies depending on the target gene;
(2) Their versatility/flexibility is low;
(3) Their generation and production is cumbersome and time consuming, especially in case of the protein-based approaches
(4) Introducing the therapeutic entity into the target cells is difficult in general and particularly in vivo, e.g., cells normally do not uptake extracellular nucleic acids or proteins
(5) In case of protein-based approaches: As these are foreign (non-self) proteins, artificial transcription factors and intrabodies might elicit an immune response, thus limiting a potential therapeutic effect.
(6) Their in vivo application meets a major hurdle in terms of delivery to the target cells in amounts high enough to provide a therapeutic benefit.

Antisense technology in particular has been the most commonly described approach in protocols to achieve down-regulation of gene expression. For antisense strategies, stochiometric amounts of single-stranded nucleic acid complementary to the messenger RNA for the gene of interest are introduced into the cell. Some difficulties with antisense-based approaches relate to delivery, stability, and dose requirements. In general, cells do not have an uptake mechanism for single-stranded nucleic acids, hence uptake of unmodified single-stranded material is extremely inefficient (see also point (4) above). Because antisense interference requires that the interfering material accumulate at a relatively high concentration (at or above the concentration of endogenous mRNA), the amount required to be delivered is a major constraint on efficacy (see also point (6) above). The use of antisense for gene therapy or other whole-organism applications has been limited by the large amounts of oligonucleotide that need to be synthesized from non-natural analogs, the cost of such synthesis, and the difficulty even with high doses of maintaining a sufficiently concentrated and uniform pool of interfering material in each cell.

(2.3) Advantages of RNAi Over Prior Approaches in General

The discovery that RNA interference (RNAi) seems to be a ubiquitous mechanism to silence genes suggests an alternative, novel approach to decrease gene expression, which is able to overcome the limitations of the other approaches outlined above. Short interfering RNAs (siRNAs) are at the heart of RNAi. The antisense strand of the siRNA is used by an RNAi silencing complex to guide cleavage of complementary mRNA molecules, thus silencing expression of the corresponding gene [6-10].

The present invention—leveraging RNAi—thus differs from other nucleic acid based strategies (antisense and ribozyme methods) in both approach and effectiveness:
(a) Compared to antisense strategies, RNAi leverages a catalytic process, i.e., a small amount of siRNA is capable of decreasing the concentration of the target gene mRNA within the target cell. As antisense is based on a stochiometric process, a much larger concentration of effector molecules is required within the target cell, i.e., a concentration is required that is equal to or greater than the concentration of endogenous mRNA. Thus, as RNAi is a catalytic process, a lower amount of effector molecules (i.e., siRNAs) is sufficient to mediate a therapeutic effect.
(b) Compared to ribozymes (which have a catalytic function as well), RNAi seems to be a more flexible strategy, which allows targeting a higher variety of target sequences and thus offers more flexibility in construct design. Moreover, design of RNAi constructs is fast and convenient as the artisan can design those constructs based on the sequence information of the RNAi target gene. With ribozymes, more trial-and-error experiments and more sophisticated design algorithms are required as ribozymes are more complex in nature. Last, RNAi is more efficacious in vivo compared to ribozymes as RNAi leverages ubiquitous, endogenous cell machinery.

The present invention also differs from protein-based strategies, as RNAi does not require the expression of non-endogenous proteins (such as artificial transcription factors), thus lowering the risk of an unintended immune response.

In summary, RNAi-mediated down-regulation of gene expression is a novel mechanism with clear advantages over existing gene expression down-regulation approaches.

(2.4) Advantages of RNAi Induced by rAAV-Mediated RNAi Expression Cassette Gene Transfer In Vivo

However, to decrease gene expression by RNAi, the siRNA molecules have to be within the target cell. Several methods have been used so far successfully in vitro such as transfection of in vitro synthesized siRNA molecules. However, these methods (based on in vitro synthesized RNA) are not highly effective in vivo for the following reasons:
(1) Due to the presence of RNAses in the extracellular milieu, RNAs have only a short half-life in vivo, which might require large amounts of RNA to be administered to a subject.
(2) Cells normally do not uptake naked RNAs or uptake naked RNA only at low rates.
(3) Naked nucleic acids outside of cells are assumed to induce autoimmune disorders and impose as such as safety concern (e.g., causing Systemic Lupus Erythematosus).
(4) Even if one succeeds in delivering the RNA to the target cell (e.g., by using liposomes), one still has to (a) readminister the RNA frequently as RNA is degraded intracellularly; (b) has to overcome the problems associated with non-viral delivery methods such as low efficiency and low cell tropism.

One first step to overcome these limitations partially, was the development of RNAi expression cassettes to mediate the expression of siRNA molecules in vivo. In that context, a gene transfer system is desirable that
(1) allows flexible targeting of a broad range of cells
(2) targets the intended target cells with (a) high specificity (e.g., through use of different serotypes), (b) high efficacy
(3) offers long-term gene expression
(4) is non-immunogenic (e.g., virus particles do not evoke an immune response)
(5) has an acceptable safety profile (e.g., non-integrating system).

Gene transfer vectors based on recombinant adeno-associated viruses (AAVs) meet all of these criteria and show great promise for in vivo gene transfer: rAAV virions can infect a broad spectrum of non-dividing cells with high efficacy and specificity (including cells of the CNS such as photoreceptor cells), are safe (replication defective, lack viral coding sequences) and induce no significant immune response to transgene products. This allows for long-term and stable gene expression [11-13].

The inventors are the first to describe the utility of AAV-mediated RNA interference in a mammalian subject in vivo by administering in vivo a recombinant adeno-associated viral gene transfer vector comprising an RNAi expression cassette. The inventors are also the first to show the usefulness of RNA Polymerase I promoters in that context. AAV-mediated RNA interference has clear advantages over other approaches for in vivo applications:
(1) AAV-mediated gene transfer allows the flexible, yet specific targeting of a broad range of cells by using alternative serotypes. More than eight AAV serotypes have been discovered so far, with each serotype having a distinct tropism. This is a clear advantage of AAV over all non-viral methods and also over retroviral gene transfer (as retroviral vectors can only transduce dividing cells).
(2) AAV-mediated gene transfer is more specific and more efficacious compared to non-viral approaches, i.e., a specific cell type can be targeted (without inadvertently transducing neighbouring cells), and transduction efficiency of the intended cell type is high.
(3) AAV offers long-term gene expression and does not induce an immune response—as compared to e.g., adenoviral vectors, which still harbor viral genes and induce an immune response.
(4) AAV vectors are relatively safe compared to retroviral or lentiviral constructs as they do not (or only to a limited extent) integrate into the host genome.

Thus, AAV-mediated RNA interference in a mammalian subject in vivo will provide useful and novel applications in at least 4 areas:
(1) Cancer therapy: siRNAs might be used to silence oncogenes [14-16]
(2) Anti-infective Therapy: siRNAs might inhibit the expression of essential viral genes or silence the expression of non-essential viral receptors [17-19], which could be used to treat infectious diseases such as virus infections (e.g., HIV) or bacterial infections.
(3) Treatment of (autosomal dominant) inherited disorders: siRNAs should be able to specifically silence mutated alleles (also in the context of gene therapy). To cure autosomal dominant diseases by gene therapy, the primary goal is not to introduce an intact copy of the mutated gene into the cells affected, but to inactivate the endogenous mutated copy, which causes the observed, undesired phenotype. Introduction of an intact copy in case of autosomal dominant mutations is only required if the patient is homozygous for the mutation, if the amount of correctly expressed protein is too low, or if the method chosen to inactivate the mutated copy also inactivates the second, non-mutated endogenous copy [20].
(4) Diseases caused by abnormal gene expression: Many diseases (such as endocrine disorders, immune disorders and so on) arise from the abnormal expression of a particular gene or group of genes within a mammal. The inhibition of the gene or group can therefore be used to treat these conditions.

(3) Description of Prior Art

(3.1) RNA Interference

Double-stranded RNA (dsRNA) can induce many different epigenetic gene-silencing processes in eukaryotes, including the degradation of homologous mRNAs—a process called RNA interference (RNAi) in animals and post-transcriptional gene silencing (PTGS) in plants. RNA interference (RNAi) has first been discovered in 1998 by Andrew Fire and Craig Mello in C. elegans, confirming former studies of PGTS in plants [21]. It now seems to be a ubiquitous mechanism—also applicable to humans [6, 7, 17, 22-29].

In both plants and animals, one key function of RNAi is to maintain genome integrity by suppressing the mobilization of transposons and the accumulation of repetitive DNA in the germ line. In plants, and perhaps also in animals, the RNAi machinery also defends cells against pathogens with double-stranded RNA genomes as part of an inborn antiviral immune response. Last, RNAi seems to regulate the expression of endogenous genes in developmental contexts [7, 29].

(3.1.1) Small RNA Species

Components of the RNAi and PTGS machinery are involved in the processing and function of different small RNA species: small interfering RNAs (siRNAs), short temporal RNAs (stRNAs) and microRNAs (miRNAs) [6, 28].

The generation of siRNAs is catalyzed by the enyzme complex Dicer [30]. Dicer recognizes the presence of dsRNA in the cytosol and catalyzes the degradation of dsRNA into 21-23 base pair (bp) dsRNA fragments (=siRNAs) with two or three 3′ overlaps on each side [31, 32]. These siRNAs subsequently function as substrates for the degradation of complementary mRNA species (RNA interference) [29].

In contrast to siRNAs, which are double-stranded and direct destruction of their target mRNAs, stRNAs are single-stranded and repress translation of their target mRNAs by binding to partially complementary sequences in the 3′-untranslated regions of their mRNA targets. stRNAs are synthesized as branch of an imperfect 70-nt RNA stemloop structure and released by the enzyme Dicer [33]. Two examples for stRNAs are lin-4 and let-7, which regulate the timing of development in C. elegans. Whereas lin-4 seems to be restricted to worms, let-7 is found more widely among animals with bilateral symmetry (including humans) [28, 34, 35].

miRNAs also exist single-stranded in vivo in many animals (from nematodes to humans). Their synthesis equals the one of stRNAs with the difference of being part of a perfect 70-nt RNA stem-loop structure. miRNAs silence genes through mRNA degradation (analogous to siRNAs; different from stRNAs) and might play a role in early development [28, 36-38].

Several groups have been reported successful siRNA-mediated knock-down of mammalian genes in tissue culture [6, 32, 39-44]. The genes targeted range from structural (e.g., lamin [32, 42]) to exogenous reporter genes (e.g., GFP [39]), confirming the hypothesis that RNAi is a ubiquitous mechanism and generally applicable to potentially any desired gene. Only recently, there have been reports of in vivo use of RNAi in mammals [39, 45, 46]: McCafrey et al. [45] were able to make a proof-of-principle in mice by co-transfecting a luciferase reporter construct together with an RNAi construct into mouse liver by hydrodynamic transfection. Similar results were obtained by Lewis et al. [46]. This group was able to silence an endogenous gene (GFP in a GFP-transgenic mouse) by hydrodynamic transfection of complementary siRNA oligonucleotides. Last, Xia et al. [39] were able to apply RNA interference in vivo in the context of a disease model (polyglutamine diseases).

(3.1.2) Mechanism of RNAi

The mechanism of RNA interference has been studied best in D. melanogaster. Researchers identified two phases: An initiation phase and an effector phase [29]:In the initiation phase, the RNAi enzyme complex Dicer recognizes dsRNA in the cytosol and catalyzes the degradation of dsRNA into 21-23 bp dsRNA fragments (=siRNAs). Structurally, Dicer contains two RNase III motifs, an RNA helicase domain, a dsRNA-binding domain (dsRBD), and a PAZ domain (PAZ: Piwi/Argonaute/Zwille). After processing, the siRNAs are integrated into the RNA-induced silencing complex (RISC), a 500 kD multiprotein complex, comprising the proteins EIF2C2, GEMIN3 and GEMIN4 in humans [6]. Within the RISC, the double-stranded siRNA is unwound, giving rise to a single-stranded guide RNA. This guide RNA is then used as a template for targeting of homologous mRNA sequences.

In the effector phase, if a complementary mRNA sequence has been found, the target mRNA will be cleaved in the middle of the annealed sequence through the RNase activity of RISC. The cleaved mRNA fragments are then released and degraded by cellular exonucleases.

It has been reported that in several lower eukaryotes an RNA-dependent polymerase amplifies the introduced dsRNA, possibly leading to a higher concentration of siRNAs [47]. Mammalian cells most likely lack this amplification mechanism. Thus, gene silencing in mammalian cells might require a much higher dosage of dsRNA compared to lower eukaryotes.

(3.1.3) Applications of RNAi

RNA interference has been successfully used to silence endogenous genes by introducing dsRNA with homology to the cellular gene transcript of interest [6, 32, 39-44]. This makes RNAi applicable in the context of basic science (reverse genetics) and medical therapy (including gene therapy) [48].

In forward genetics, one first isolates a mutant with a specific phenotype and then tries to identify the gene(s) involved; with reverse genetics, one starts with a specific gene of interest, downregulates its expression, and looks for phenotypic changes. Before the discovery of RNAi, downregulation was primarily achieved by knocking-out genes of interest through homologous recombination, a tedious process, which is difficult to upscale. With (inducible) RNAi as a new tool, this process can be standardized and even industrialized, allowing to downregulate each potential gene identified through the sequencing of the human genome for functional studies—without the need for homologous recombination or potentially even germ-line transmission [48].

The focus of the present invention is on medical applications of RNAi via AAV-mediated RNAi expression cassette transfer to a mammalian subject in vivo. Here, four areas might prove ideal candidates for leveraging RNAi:
(1) Cancer therapy: siRNAs might be used to silence oncogenes [14-16]
(2) Anti-infective Therapy: siRNAs might inhibit the expression of essential viral genes or silence the expression of non-essential viral receptors [17-19], which could be used to treat infectious diseases such as virus infections (e.g., HIV) or bacterial infections.
(3) Treatment of autosomal dominant inherited disorders: siRNAs should be able to specifically silence mutated alleles (also in the context of gene therapy). To cure autosomal dominant diseases by gene therapy, the primary goal is not to introduce an intact copy of the mutated gene into the cells affected, but to inactivate the endogenous mutated copy, which causes the observed, undesired phenotype. Introduction of an intact copy in case of autosomal dominanmutations is only required if the patient is homozygous for the mutation, if the amount of correctly expressed protein is too low, or if the method chosen to inactivate the mutated copy also inactivates the second, non-mutated endogenous copy [20].
(4) Diseases caused by abnormal gene expression: Many diseases (such as endocrine disorders, immune disorders and so on) arise from the abnormal expression of a particular gene or group of genes within a mammal. The inhibition of the gene or group can therefore be used to treat these conditions.

(3.2) Gene Transfer

Gene transfer systems can be classified along different dimensions

(A) Nature or origin of the system

(B) Delivery mechanism

(C) Site of gene transfer (e.g., ex vivo, in vitro, in vivo)

to (A): Based on the nature or origin of the gene transfer system, existing delivery systems for nucleic acid compositions can be subdivided into three groups: (1) viral vectors, (2) non-viral vectors, and (3) naked nucleic acids. Regarding vector targeting (specificity) and efficiency, viral vector systems are superior to conventional non-viral vectors and naked nucleic acids. On the other hand, non-viral vectors and naked nucleic acids are safer, easier to upscale in production and allow for the delivery of modified nucleic acids compared to viral vectors.

to (B): Alternatively, based on the delivery mechanism, gene transfer methods fall into the following three broad categories: (1) physical (e.g., electroporation, direct gene transfer and particle bombardment), (2) chemical (e.g. lipid-based carriers and other non-viral vectors) and (3) biological (e.g. virus or bacterium derived vectors).

to (C): Gene therapy methodologies can also be described by delivery site. Fundamental ways to deliver genes include ex vivo gene transfer, in vivo gene transfer, and in vitro gene transfer. In ex vivo gene transfer, cells are taken from the subject and grown in cell culture. The nucleic acid composition is introduced into the cells, the transduced or transfected cells are (in some instances) expanded in number and then reimplanted in the subject. In in vitro gene transfer, the transformed cells are cells growing in culture, such as tissue culture cells, and not particular cells from a particular subject. These “laboratory cells” are transfected or transduced; the transfected or transduced cells are then in some instances selected and/or expanded for either implantation into a subject or for other uses. In vivo gene transfer involves introducing the nucleic acid composition into the cells of the subject when the cells are within the subject.

Several delivery mechanisms may be used to achieve gene transfer in vivo, ex vivo, and/or in vitro.

Mechanical (i.e. physical) methods of DNA delivery can be achieved by direct injection of DNA, such as microinjection of DNA into germ or somatic cells, pneumatically deivered DNA-coated particles, such as the gold particles used in a “gene gun,” and inorganic chemical approaches such as calcium phosphate transfection. It has been found that physical injection of plasmid DNA into muscle cells yields a high percentage of cells that are transfected and have a sustained expression of marker genes. The plasmid DNA may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products. Particle-mediated gene transfer may also be employed for injecting DNA into cells, tissues and organs. With a particle bombardment device, or “gene gun,” a motive force is generated to accelerate DNA coated high-density particles (such as gold or tungsten) to a high velocity that allows penetration of the target organs, tissues or cells. Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. The techniques of particle-mediated gene transfer and electroporation are well known to those of ordinary skill in the art.

Chemical methods of gene therapy involve carrier mediated gene transfer through the use of fusogenic lipid vesicles such as liposomes or other vesicles for membrane fusion. A carrier harboring a DNA of interest can be conveniently introduced into body fluids or the bloodstream and then site specifically directed to the target organ or tissue in the body. Liposomes, for example, can be developed which are cell specific or organ specific. The foreign DNA carried by the liposome thus will be taken up by those specific cells. Injection of immunoliposomes that are targeted to a specific receptor on certain cells can be used as a convenient method of inserting the DNA into the cells bearing the receptor. Another carrier system that has been used is the asialoglycoprotein/polylysine conjugate system for carrying DNA to hepatocytes for in vivo gene transfer. Transfected DNA may also be complexed with other kinds of carriers so that the DNA is carried to the recipient cell and then resides in the cytoplasm or in the nucleoplasm of the recipient cell. DNA can be coupled to carrier nuclear proteins in specifically engineered vesicle complexes and carried directly into the nucleus. Carrier mediated gene transfer may also involve the use of lipid-based proteins which are not liposomes. For example, lipofectins and cytofectins are lipid-based positive ions that bind to negatively charged DNA, forming a complex that can ferry the DNA across a cell membrane. Another method of carrier mediated gene transfer involves receptor-based endocytosis. In this method, a ligand (specific to a cell surface receptor) is made to form a complex with a gene of interest and then injected into the bloodstream; target cells that have the cell surface receptor will specifically bind the ligand and transport the ligand-DNA complex into the cell.

Biological gene therapy methodologies usually employ viral vectors to insert genes into cells. The transduced cells may be cells derived from the patient's normal tissue, the patient's diseased tissue, or may be non-patient cells. Viral vectors that have been used for gene therapy protocols include but are not limited to, retroviruses, lentivruses, other RNA viruses such as pol1ovirus or Sindbis virus, adenovirus, adeno-associated virus, herpes viruses, simian virus 40, vaccinia and other DNA viruses.

Replication-defective murine retroviral vectors are commonly utilized gene transfer vectors. Murine leukemia retroviruses are composed of a single strand RNA complex with a nuclear core protein and polymerase (pol) enzymes, encased by a protein core (gag) and surrounded by a glycoprotein envelope (env) that determines host range. The genomic structure of retroviruses include the gag, pol, and env genes flanked by 5′ and 3′ long terminal repeats (LTR). Retroviral vector systems exploit the fact that a minimal vector containing the 5′ and 3′ LTRs and the packaging signal are sufficient to allow vector packaging, infection and integration into target cells providing that the viral structural proteins are supplied in trans in the packaging cell line. Fundamental advantages of retroviral vectors for gene transfer include efficient infection and gene expression in most dividing cell types, precise single copy vector integration into target cell chromosomal DNA, and ease of manipulation of the retroviral genome. For example, altered retrovirus vectors have been used in ex vivo and in vitro methods to introduce genes into peripheral and tumor-infiltrating lymphocytes, hepatocytes, epidermal cells, myocytes, or other somatic cells (which may then be introduced into the patient to provide the gene product from the inserted DNA). For descriptions of various retroviral systems, see, e.g., U.S. Pat. No. 5,219,740; [49-53]. The main disadvantage of retroviral systems is that retroviral vectors can only infect dividing cells. Lentiviral vectors overcome this limitation. Nevertheless, production of retro- and lentiviral vectors is complex, and the virions are not very stable compared to other viruses. More recently, the danger of inducing cancer through insertional mutagenesis has been raised as a major safety concern [54] [55].

A number of adenovirus based gene delivery systems have also been developed. Human adenoviruses are double stranded, linear DNA viruses with a protein capsid that enter cells by receptor-mediated endocytosis. Adenoviral vectors have a broad host range and are highly infectious, even at low virus titers. Moreover, adenoviral vectors can accommodate relatively long transgenes compared to other systems. A number of adenovirus based gene delivery systems have also been described [56-62]. The main limitation of adenoviral vectors is their high degree of immunogenicity, which limits their use in respect to applications that require long-term gene expression.

For many applications, long-term gene expression (over several years) will have to be achieved. This is also the case for the present invention. So far, primarily adeno-associated virus based vectors allow for this. Most other viral vectors are limited by expression of viral genes so that transduced cells will be eliminated by the immune system (e.g., adenoviral vectors), gene silencing (retroviral vectors or lentiviral vectors) or questionable safety profile (e.g., retroviral vectors or adenoviral vectors).

(3.2.1) Adeno-Associated Viral Vectors

The present invention uses adeno-associated virus-based vectors [63] [64] [65] for the transfer of an RNAi expression cassette into the appropriate target cells of a mammalian subject in vivo.

Adeno associated virus (AAV) is a small nonpathogenic virus of the parvoviridae family. AAV is distinct from the other members of this family by its dependence on a helper virus for replication. The approximately 5 kb genome of AAV consists of single stranded DNA of either plus or minus polarity. The ends of the genome are short inverted terminal repeats (ITRs), which can fold into hairpin structures and serve as the origin of viral DNA replication. Physically, the parvovirus virion is non-enveloped and its icosohedral capsid is approximately 20 nm in diameter. To date, at least 8 serologically distinct AAVs have been identified and isolated from humans or primates and are referred to as AAV types 1-8. The most extensively studied of these isolates are AAV type 2 (AAV2) and AAV type 5 (AAV5).

The genome of AAV2 is 4680 nucleotides in length and contains two open reading frames (ORFs). The left ORF encodes the non-structural Rep proteins, Rep40, Rep52, Rep68 and Rep78, which are involved in regulation of replication and transcription in addition to the production of single-stranded progeny genomes. Furthermore, two of the Rep proteins have been associated with the preferential integration of AAV2 genomes into a region of the q arm of human chromosome 19. Rep68/78 have also been shown to possess NTP binding activity as well as DNA and RNA helicase activities. The Rep proteins possess a nuclear localization signal as well as several potential phosphorylation sites. Mutation of one of these kinase sites resulted in a loss of replication activity. The ends of the genome are short inverted terminal repeats, which have the potential to fold into T-shaped hairpin structures that serve as the origin of viral DNA replication. Within the ITR region two elements have been described which are central to the function of the ITR, a GAGC repeat motif and the terminal resolution site (trs). The repeat motif has been shown to bind Rep when the ITR is in either a linear or hairpin conformation. This binding serves to position Rep68/78 for cleavage at the trs, which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The right ORF of AAV2 encodes related capsid proteins referred to as VP1, 2 and 3. These capsid proteins form the icosahedral, non-enveloped virion particle of ˜20 nm diameter. VP1, 2 and 3 are found in a ratio of 1:1:10. The capsid proteins differ from each other by the use of alternative splicing and an unusual start codon. Deletion analysis has shown that removal or alteration of VP1, which is translated from an alternatively spliced message, results in a reduced yield of infectious particles. Mutations within the VP3 coding region result in the failure to produce any single-stranded progeny DNA or infectious particles.

The findings described in the context of AAV2 are generally applicable to other AAV serotypes as well.

The following features of AAV have made it an attractive vector for gene transfer. AAV vectors possess a broad host range [66], transduce both dividing and non dividing cells in vitro and in vivo and maintain high levels of expression of the transduced genes in the absence of a significant immune response to the transgene product in general. Moreover, as wild-type AAV is non-pathogenic, AAV vector particles are assumed to be non-pathogenic as well (in contrast to adenoviral vectors). Viral particles are heat stable, resistant to solvents, detergents, changes in pH and temperature. The ITRs have been shown to be the only cis elements required for replication and packaging and may contain some promoter activities. Thus, AAV vectors encode no viral genes.

### SUMMARY OF INVENTION

(1) Substance or General Idea of the Claimed Invention

The present invention provides a method for decreasing or down-regulating gene expression at the mRNA level in a cell of a mammalian subject in vivo. The method involves administering to a (cell of a) mammalian subject in vivo a recombinant adeno-associated viral vector with said vector comprising an RNA interference (RNAi) expression cassette whose RNA expression product(s) directly or indirectly lead to a decrease in expression of the corresponding RNAi target gene by forming a double-stranded RNA complex which induces “RNA mediated interference” or “RNA interference” (“RNAi”), a post-transcriptional gene silencing mechanism. The dsRNA complex comprises a nucleotide sequence that hybridizes under physiologic conditions of the cell to the nucleotide sequence of at least a portion of the mRNA transcript of the gene to be down-regulated (i.e., the RNAi target gene). In particular, the RNA expression products of the RNAi expression cassette will decrease the cellular concentration of the mRNA transcript of the RNAi target gene, thus resulting in decreased concentration of the protein encoded by the RNAi target gene in the mammalian subject. Down-regulation of gene expression is specific in that a nucleotide sequence from a portion of the RNAi target gene is chosen in designing the sequence properties of the RNA coding region of the RNAi expression cassette to be transferred via rAAV-mediated gene transfer into the cells of a mammalian subject in vivo; or alternatively said: Inhibition is sequence-specific in that nucleotide sequences corresponding to the duplex region of the double-stranded RNA complex are targeted for RNA interference.

We disclose that the method of the present invention (1) Is effective in decreasing or down-regulating gene expression in a mammalian subject in vivo; (2) allows decreasing of gene expression of many different types of RNAi target genes; (3) allows decreasing of gene expression in many different cell types, tissues, and organs of a mammalian subject in vivo; (4)Allows decreasing of gene expression via rAAV-mediated RNAi expression cassette gene transfer to a mammalian subject in vivo using a multitude of RNAi expression cassette designs.

A significant aspect of the present invention relates to the demonstration that RNAi can in fact be accomplished in vivo in mammalian subjects by AAV-mediated gene transfer of an RNAi expression cassette. This had not been previously described in the art. Thus, the present invention provides, for the first time, a demonstration of the application of the RNAi technique in a mammalian subject in vivo using adeno-associated viral vectors: Upon successful in vivo transduction with the recombinant adeno-associated viral vector, the RNA expression products of the RNAi expression cassette will decrease the cellular concentration of the mRNA transcript of the RNAi target gene, thus resulting in decreased concentration of the protein encoded by the RNAi target gene in the mammalian subject.

Also disclosed are pharmaceutical kits containing the rAAV vector in a suitable pharmaceutical suspension for administration. In this aspect, the invention provides a pharmaceutical kit for delivery of said recombinant adeno-associated viral vector or virion. The kit may contain a container for administration of a predetermined dose. The kit further may contain a suspension containing the gene transfer vector or virion for delivery of a predetermined dose, said suspension comprising (a)the rAAV gene transfer vector or virion comprising an RNAi expression cassette(b)a physiologically compatible carrier.

In another aspect, the present invention relates to methods of controlling the expression of known genes or known nucleic acid sequences in mammalian cells in vivo by expressing sense and antisense RNA sequences (with respect to the gene or nucleic acid sequence) capable of forming double-stranded RNA complexes and inducing RNAi. In that context, the RNA molecules are expressed by administering in vivo a recombinant adeno-associated viral vector comprising an RNAi expression cassette encoding said RNA molecule(s). Thus, the invention also relates to rAAV-mediated expression of RNA molecules for forming dsRNA complexes, to DNA molecules (e.g., RNAi expression cassettes) encoding the RNA molecules for forming dsRNA complexes, to rAAV vectors and cells comprising such molecules, to rAAV virions comprising such rAAV vectors, to compositions comprising said rAAV virions, and to prophylactic and therapeutic methods for administering said rAAV vectors or virions.

The invention also provides RNAi expression cassettes that encode the RNA molecule(s) capable of forming a double-stranded RNA complex and thus capable of inducing RNA interference. Such RNAi expression cassettes may be a single DNA molecule as part of a rAAV genome which, when introduced into a cell, gives rise to a single RNA molecule capable of forming intramolecularly a dsRNA complex. However it will be understood from the following description that more than one rAAV genomes or RNAi expression cassettes or RNA coding regions may be introduced into a cell, either simultaneously or sequentially, to give rise to two or more RNA molecules capable of forming intermolecularly a dsRNA complex. Typically, the two RNA moieties capable of forming a dsRNA complex, whether intra- or intermolecularly, are at least in part sense and at least in part antisense sequences of a gene or nucleic acid sequence whose expression is to be down-regulated or decreased.

The design of the RNAi expression cassette does not limit the scope of the invention. Different strategies to design an RNAi expression cassette can be applied, and RNAi expression cassettes based on different designs will be able to induce RNA interference in vivo. (Although the design of the RNAi expression cassette does not limit the scope of the invention, some RNAi expression cassette designs are included in the detailed description of this invention and below. ) Features common to all RNAi expression cassettes are that they comprise an RNA coding region which encodes an RNA molecule which is capable of inducing RNA interference either alone or in combination with another RNA molecule by forming a double-stranded RNA complex either intramolecularly or intermolecularly.

Different design principles can be used to achieve that same goal and are known to those of skill in the art. For example, the RNAi expression cassette may encode one or more RNA molecules. After or during RNA expression from the RNAi expression cassette, a double-stranded RNA complex may be formed by either a single, self-complementary RNA molecule or two complementary RNA molecules. Formation of the dsRNA complex may be initiated either inside or outside the nucleus.

In one aspect there is provided a double-stranded RNA complex, which comprises, a first RNA portion capable of hybridizing under physiological conditions to at least a portion of an mRNA molecule, and a second RNA portion wherein at least a part of the second RNA portion is capable of hybridizing under physiological conditions to the first portion. Preferably the first and second portions are part of the same RNA molecule and are capable of hybridization at physiological conditions, such as those existing within a cell, and upon hybridization the first and second portions form a double-stranded RNA complex.

In another aspect there is provided a linear RNA molecule for forming a double-stranded RNA complex, which RNA comprises a first portion capable of hybridizing to at least a portion of an mRNA molecule, preferably within a cell and a second portion wherein at least part of the second portion is capable of hybridizing to the first portion to form a hairpin dsRNA complex.

In yet another aspect, the method comprises AAV-mediated expression of RNA with partial or fully double-stranded character in vivo.

A dsRNA complex containing a nucleotide sequence identical to a portion of the RNAi target gene is preferred for inhibition. RNA sequences with insertions, deletions, and single point mutations relative to the RNAi target sequence have also been found to be effective for inhibition. Thus, sequence identity may be optimized by alignment algorithms known in the art and calculating the percent difference between the nucleotide sequences. Alternatively, the duplex region of the dsRNA complex may be defined functionally as a nucleotide sequence that is capable of hybridizing with a portion of the target gene transcript.

In the preferred embodiment the RNAi expression cassette comprises at least one RNA coding region. Preferably the RNA coding region is a DNA sequence that can serve as a template for the expression of a desired RNA molecule in the host cell. In one embodiment, the RNAi expression cassette comprises two or more RNA coding regions. The RNAi expression cassette also preferably comprises at least one RNA Polymerase III promoter. The RNA Polymerase III promoter is operably linked to the RNA coding region, and the RNA coding region can also be linked to a terminator sequence. In addition, more than one RNA Polymerase III promoters may be incorporated.

In certain embodiments the invention employs ribozyme-containing RNA molecules to generate dsRNA complexes, thereby overcoming certain known difficulties associated with generating dsRNA. For example, the ribozyme functionality might be used to remove polyadenylation signals, thus preventing or minimizing release of the RNA molecule from the nucleus of a cell. In other embodiments the invention is based on the ability of a portion of the RNA molecule to encode an RNA or protein that enhances specific activity of dsRNA. One example of this specific activity-enhancing portion of the RNA molecule is a portion of the molecule encoding the HIV Tat protein to inhibit the cellular breakdown of dsRNA complexes. Such a portion is additionally useful in treating disorders such as HIV infection.

In another aspect of the invention, expression of the RNA coding region results in the down regulation of a target gene. Preferably the target gene comprises a sequence that is at least about 90% identical with the RNA coding region, more preferably at least about 95% identical, and even more preferably at least about 99% identical.

The RNAi target gene does not limit the scope of this invention and may be any gene derived from the cell: an endogenous gene, a transgene, or a gene of a pathogen that is present in the cell after infection thereof. Thus, the choice of the RNAi target gene is not limiting for the present invention: The artisan will know how to design an RNAi expression cassette to down-regulate the gene expression of any RNAi target gene of interest. Depending on the particular RNAi target gene and the dose of rAAV virions delivered, the procedure may provide partial or complete loss of function for the RNAi target gene.

Additionally, the RNAi target cell to be transduced in vivo does not limit the scope of this invention and may be from the germ line or somatic, totipotent or pluripotent, dividing or non-dividing, parenchyma or epithelium, immortalized or transformed, or the like. The RNAi target cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. The RNAi target cell might be a muscle cell, a liver cell, a lung cell or a brain cell. In its preferred embodiment, the RNAi target cell is a photoreceptor cell of the retina.

Moreover, the use of a specific AAV serotype does not limit the scope of this invention. Different AAV serotypes can be used to transduce different types of cells, and the tissue tropism of different AAV serotypes are known to those of skill in the art or can be determined by the artisan without undue effort. Thus, the artisan will choose the most appropriate AAV serotype for the transfer of an RNAi expression cassette into the corresponding RNAi target cell type.

According to a further aspect of the invention, the rAAV vector may also comprise a nucleotide sequence encoding a gene of interest. The gene of interest is preferably operably linked to a Polymerase II promoter. Such a construct also can contain, for example, an enhancer sequence operably linked with the Polymerase II promoter. The gene of interest is not limited in any way and includes any gene that the skilled practitioner desires to have expressed. For example, the gene of interest may be one that encodes a protein that serves as a marker to identify transduced cells. In other embodiments the gene of interest encodes a protein that has a therapeutic or palliative effect on the mammalian subject. In addition, more than one gene of interest may be included in the rAAV vector. For example a gene encoding a marker protein may be placed after the primary gene of interest to allow for identification of cells that are expressing the desired protein.

The gene of interest could encode any variety of proteins including, but not limited to viral proteins capable of modulating the global mammalian cell response to dsRNA, and would include but not be restricted to, mammalian viral proteins (vaccinia virus early protein E3L, reovirus p3 protein, vaccinia virus pK3, HIV-1 Tat) or cellular proteins (PKR dominant negative proteins, p58, and oncogenes such as v-erbB, sos or activated ras). In addition the gene of interest could encode any enzyme component of the host protein complex that acts specifically on dsRNA to enhance the efficacy of the dsRNA in controlling specific gene expression. In a preferred embodiment the protein that enhances the specific activity of dsRNA would be the HIV Tat protein. Moreover, the gene of interest might encode proteins involved in RNA interference within a cell, e.g., Argonaut proteins or Dicer proteins.

In one embodiment, the RNAi target gene is the Rhodopsin gene and the gene of interest is a version of the Rhodopsin transgene (cDNA) with silent point mutations in the RNAi target sequence so that this Rhodopsin gene version with silent point mutations will not be subject to the RNA interference induced by rAAV-mediated transfer of such an RNAi expression cassette.

In another embodiment a fluorescent marker protein, preferably green fluorescent protein (GFP), is incorporated into the construct along with the gene of interest. If a second reporter gene is included, an internal ribosomal entry site (IRES) sequence is also preferably included.

In yet another embodiment, the gene of interest is a gene included for safety concerns to allow for the selective killing of the transduced RNAi target cells within a heterogeneous population, for example within a mammal, or more particularly within a human patient. In one such embodiment, the gene of interest is a thymidine kinase gene (TK) the expression of which renders a target cell susceptible to the action of the drug gancyclovir.

Practice of the present invention will provide useful medical applications as described below under “Utility Of The Present Invention”. Moreover, this discovery of the value of AAV-gene transfer mediated RNAi for down-regulating, decreasing or inhibiting mammalian gene expression offers a tool for developing new strategies for blocking gene function, and for producing AAV-based RNAi vectors to treat human disease. The invention provides the method, wherein the cells are mammalian cells, and in one embodiment the cells are human. In the present invention, the double-stranded RNA complex expressed by the RNAi expression cassette transferred into the target cell via a rAAV vector can be used to inhibit a target gene which causes or is likely to cause disease, i.e. it can be used for the treatment or prevention of disease. In the prevention of disease, the RNAi target gene may be one which is required for initiation or maintenance of the disease, or which has been identified as being associated with a higher risk of contracting the disease. Thus, the invention provides a method for treating a mammalian subject with a genetic disorder or disease caused by overexpression of a gene or by expression of a mutated gene by administering to the mammalian subject in vivo a rAAV vector comprising an RNAi expression cassette for initiating down-regulation of the RNAi target gene expression at the mRNA level, wherein the method comprises using RNAi to achieve post-transcriptional gene silencing. In this embodiment, the preferred mammalian subject is a human patient. An embodied target cell in the method of the invention is a pathogen infected cell or a tumor cell, and the tumor cell may be malignant. In another preferred embodiment, the target cell is a photoreceptor cell, and the RNAi target gene is the Rhodopsin gene. Moreover, in these embodiments, as in the method above, the method further comprises initiating RNAi, wherein the dsRNA complex is specific for the intended RNAi target gene.

In another aspect the invention provides a mammalian cell in which a specified gene or a specified nucleic acid sequence has been suppressed by a method of the present invention.

In yet another aspect the invention provides a method of modulating expression of a gene or a nucleic acid sequence in mammalian cells including exposing said cells to recombinant adeno-associated viral vectors in vivo.

In another aspect the present invention provides a method of modulating a cellular response wherein said response is due either directly or indirectly to the expression of a gene or nucleic acid sequence and wherein expression of said gene or nucleic acid sequence is suppressed by a method of the present invention.

In a further aspect the present invention provides a method of treating a disorder resulting either directly or indirectly from expression of a gene or nucleic acid sequence wherein expression of said gene or nucleic acid sequence is suppressed by a method of the present invention.

This invention also provides a method of treating a subject having a disorder ameliorated by inhibiting the expression of a known gene in the subject's cells, comprising administering to the subject a therapeutically effective amount of the instant pharmaceutical compositions comprising rAAV virions comprising RNAi expression cassette(s) encoding (at least) one RNA molecule which is capable of forming a dsRNA complex wherein, under hybridizing conditions, the a portion of the dsRNA complex is able to hybridize to at least a portion of an mRNA encoded by the gene whose expression is to be inhibited.

This invention also provides a method of inhibiting in a subject the onset of a disorder ameliorated by inhibiting the expression of a known gene in the subject's cells, comprising administering to the subject a prophylactically effective amount of the instant pharmaceutical composition comprising rAAV virions comprising RNAi expression cassette(s) encoding (at least) one RNA molecule which is capable of forming a dsRNA complex wherein, under hybridizing conditions, the a portion of the dsRNA complex is able to hybridize to at least a portion of an mRNA encoded by the gene whose expression is to be inhibited.

(2) Advantages of the Invention Over Prior Approaches

AAV-mediated transfer of RNAi expression cassettes in vivo represents a useful, novel and non-obvious advancement. Prior approaches to induce RNAi in vivo include (1) direct transfection of RNA; (2) transfection with plasmids or generally DNA comprising an RNAi expression cassette; (3) use of lentiviral or adenoviral vectors. However, all these prior approaches reveal significant shortcomings when compared to rAAV-mediated transfer of RNAi expression cassettes.

to (1): Direct in vivo transfection of in vitro synthesized RNA is not highly effective in vivo for the following reasons:
(a) Due to the presence of RNAses in the extracellular milieu, RNAs have only a short half-life in vivo, which might require large amounts of RNA to be administered to a subject.
(b) Cells normally do not uptake naked RNAs or uptake naked RNA only at low rates.
(c) Even if one succeeds in delivering the RNA to the target cell (e.g., by using liposomes), one still has to readminister the RNA frequently as RNA is degraded intracellularly and to overcome the problems associated with non-viral delivery methods such as low efficiency and low cell tropism.

One first step to overcome these limitations partially, was the development of RNAi expression cassettes to mediate the expression of siRNA molecules in vivo. In that context, a gene transfer system is desirable that (1) allows flexible targeting of a broad range of cells; (2) targets the intended target cells with (a) high specificity (e.g., through use of different serotypes), (b) high efficacy; (3) offers long-term gene expression; (4) is non-immunogenic (e.g., virus particles do not evoke an immune response); (5) has an acceptable safety profile (e.g., non-integrating system).

Gene transfer vectors based on recombinant adeno-associated viruses (AAVs) meet all of these criteria and show great promise for in vivo gene transfer: rAAV vectors can infect a broad spectrum of non-dividing cells with high efficacy and specificity (including cells of the CNS such as photoreceptor cells), are safe (replication defective, lack viral coding sequences) and induce no significant immune response to transgene products. This allows for long-term and stable siRNA expression [11-13].

The inventors are the first to describe the utility of AAV-mediated RNA interference in a mammalian subject in vivo by administering in vivo a recombinant adeno-associated viral gene transfer vector comprising an RNAi expression cassette. AAV-mediated RNA interference has clear advantages over other approaches for in vivo applications:
(1) AAV-mediated gene transfer allows the flexible, yet specific targeting of a broad range of cells by using alternative serotypes. More than eight AAV serotypes have been discovered so far, with each serotype having a distinct tropism. This is a clear advantage of AAV over all non-viral methods and also over retroviral gene transfer (as retroviral vectors can only transduce dividing cells).
(2) AAV-mediated gene transfer is more specific and more efficacious compared to non-viral approaches, i.e., a specific cell type can be targeted (without inadvertently transducing neighbouring cells), and transduction efficiency of the intended cell type is high.
(3) AAV offers long-term gene expression and does not induce an immune response—as compared to e.g., adenoviral vectors, which still harbor viral genes and induce an immune response.
(4) AAV vectors are relatively safe compared to retroviral or lentiviral constructs as they do not (or only to a limited extent) integrate into the host genome.

(3.) Utility of the Present Invention

AAV-mediated RNA interference in a mammalian subject in vivo will provide useful and novel applications in at least 4 areas:
(1) Cancer therapy: siRNAs might be used to silence oncogenes [14-16]
(2) Anti-infective Therapy: siRNAs might inhibit the expression of essential viral genes or silence the expression of non-essential viral receptors [17-19], which could be used to treat infectious diseases such as virus infections (e.g., HIV) or bacterial infections.
(3) Treatment of (autosomal dominant) inherited disorders: siRNAs should be able to specifically silence mutated alleles (also in the context of gene therapy), an area, we would like to pursue with our grant application. To cure autosomal dominant diseases by gene therapy, the primary goal is not to introduce an intact copy of the mutated gene into the cells affected, but to inactivate the endogenous mutated copy, which causes the observed, undesired phenotype. Introduction of an intact copy in case of autosomal dominant mutations is only required if the patient is homozygous for the mutation, if the amount of correctly expressed protein is too low, or if the method chosen to inactivate the mutated copy also inactivates the second, non-mutated endogenous copy [20].
(4) Diseases caused by abnormal gene expression: Many diseases (such as endocrine disorders, immune disorders and so on) arise from the abnormal expression of a particular gene or group of genes within a mammal. The inhibition of the gene or group can therefore be used to treat these conditions.

In one aspect, the methods of the invention relate to the treatment or prevention of infection through the rAAV-mediated expression of one or more RNA molecules that inhibit one or more aspects of the life cycle of a pathogen through RNA interference with a target nucleic acid, such as a viral genome, a viral transcript or a host cell gene that is necessary for viral replication. The RNA coding region preferably comprises a sequence that is at least about 90% identical to a target sequence within the target nucleic acid. Preferably the target nucleic is necessary for the life cycle of a pathogen, for example, part of a pathogenic virus RNA genome or genome transcript, or part of a target cell gene involved in the life cycle of a pathogenic virus. In a particular embodiment the methods are used to disrupt the life cycle of a virus having an RNA genome, for example a retrovirus or lentivirus, by targeting the RNA genome directly. In another embodiment a viral genome transcript is targeted, including transcripts of individual viral genes. The methods also can be used to down-regulate a gene in a host cell, where the gene is involved in the viral life cycle, for example, a receptor or coreceptor necessary for viral entry into the host cell. According to the invention, one of skill in the art can target a cellular component, either an RNA or an RNA encoding a cellular protein necessary for the pathogen life cycle, particularly a viral life cycle. In a preferred embodiment, the cellular target chosen will not be a protein or RNA that is necessary for normal cell growth and viability. Suitable proteins for disrupting the viral life cycle include, for example, cell surface receptors involved in viral entry, including both primary receptors and secondary receptors, and transcription factors involved in the transcription of a viral genome, proteins involved in integration into a host chromosome, and proteins involved in translational or other regulation of viral gene expression.

A number of cellular proteins are known to be receptors for viral entry into cells. Some such receptors are listed in an article by E. Baranowski, C. M. Ruiz-Jarabo, and E. Domingo, “Evolution of Cell Recognition by Viruses,” Science 292: 1102-1105, which is hereby incorporated by reference in its entirety. Some cellular receptors that are involved in recognition by viruses are listed below: Adenoviruses: CAR, Integrins, MHC I, Heparan sulfate glycoaminoglycan, Siliac Acid; Cytomegalovirus: Heparan sulfate glycoami noglycan; Coxsackieviruses: Integrins, ICAM-1, CAR, MHC I; Hepatitis A: murine-like class I integral membrane clycoprotein; Hepatitis C: CD81, Low density lipoprotein receptor; HIV (Retroviridae): CD4, CXCR4, Heparan sulfate glycoaminoglycan; HSV: Heparan sulfate glycoaminoglycan, PVR, HveB, HveC; Influenza Virus: Sialic acid; Measles: CD46, CD55; Pol1ovirus,: PVR, HveB, HveC; Human papillomavirus: Integrins. One of skill in the art will recognize that the invention is not limited to use with receptors that are currently known. As new cellular receptors and coreceptors are discovered, the methods of the invention can be applied to such sequences.

The methods of the invention can be used to treat a variety of viral diseases, including, for example, human immunodeficiency virus (HIV-1 and HIV-2), hepatitis A, hepatitis B, hepatitis C. The invention also includes methods of treating a patient having a viral infection. In one embodiment the method comprises administering to the patient an effective amount of a recombinant AAV particle (or particles) encoding at least one double stranded RNA having at least 90% homology and preferably identical to a region of at least about 15 to 25 nucleotides in a nucleotide that is important for normal viral replication. For example, the dsRNA complex may have homology to a nucleic acid in a viral genome, a viral gene transcript or in a gene for a patient's cellular receptor that is necessary for the life cycle of the virus.

Other aspects and advantages of the invention will be readily apparent to one of skill in the art from the detailed description of the invention. Additional objects, advantages and novel features of the invention will be set forth in part in the description, examples and embodiments which follow, and in part will become apparent to those skilled in the art on examination of the following, or may be learned by practice of the invention.

### BRIEF DESCRIPTION OF DRAWINGS

FIG. 1

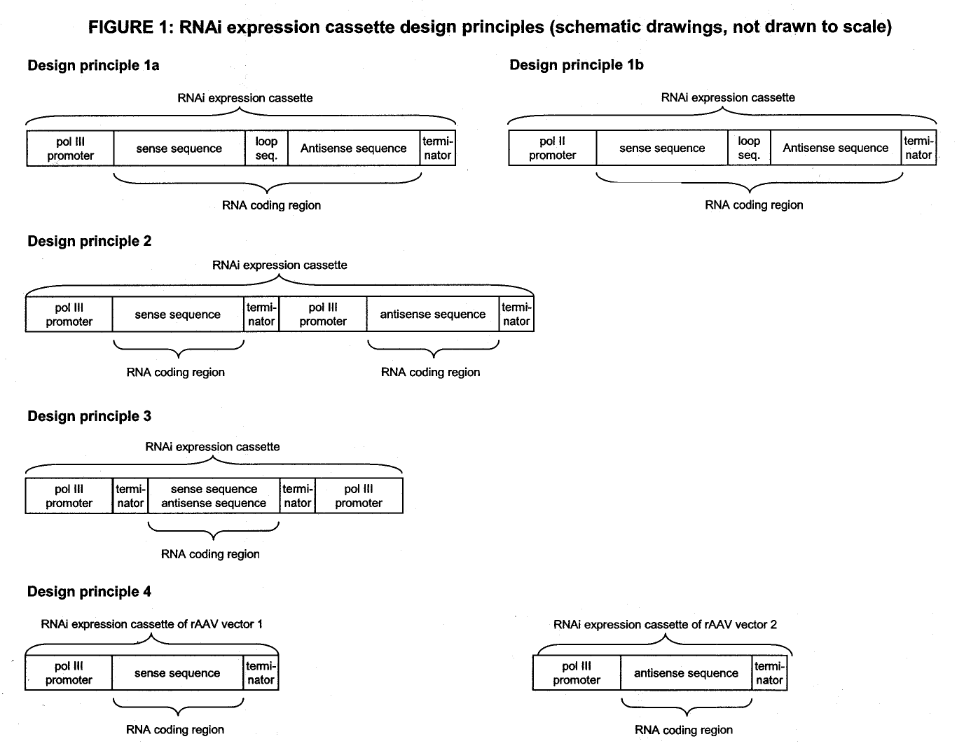

Design principle 1a: An rAAV vector comprising an RNAi expression cassette encoding a single RNA molecule capable of forming an RNAi inducing dsRNA complex intramolecularly (based on pol III promoter).

Design principle 1b: An rAAV vector comprising an RNAi expression cassette encoding a single RNA molecule capable of forming an RNAi inducing dsRNA complex intramolecularly (based on pol II promoter)

Design principle 2: An rAAV vector comprising an RNAi expression cassette comprising two RNA coding regions with each region encoding one RNA molecule with the two RNA molecules (encoded by the two different RNA coding regions) combined capable of forming an RNAi inducing dsRNA complex intermolecularly.

Design principle 3: An rAAV vector comprising an RNAi expression cassette comprising one RNA coding region transcribed into a sense and antisense RNA molecule with the sense and antisense RNA molecule combined capable of forming an RNAi inducing dsRNA complex intermolecularly.

Design principle 4: Two rAAV vectors each comprising an RNAi expression cassette with the first rAAV vector encoding a sense RNA molecule, the second rAAV vector encoding a (complementary) antisense RNA molecule with both RNA molecules combined (when expressed in same cell) capable of forming an RNAi inducing dsRNA complex intermolecularly.

### DETAILED DESCRIPTION

The present invention provides a method for decreasing or down-regulating gene expression at the mRNA level in a cell of a mammalian subject in vivo. The method involves administering to a (cell of a) mammalian subject in-vivo a recombinant adeno-associated viral vector with said vector comprising an RNA interference (RNAi) expression cassette whose RNA expression product(s) directly or indirectly lead to a decrease in expression of the corresponding RNAi target gene by forming a double-stranded RNA complex which induces “RNA mediated interference” or “RNA interference” (“RNAi”), a post-transcriptional gene silencing mechanism. The dsRNA complex comprises a nucleotide sequence that hybridizes under physiologic conditions of the cell to the nucleotide sequence of at least a portion of the mRNA transcript of the gene to be down-regulated (i.e., the RNAi target gene). In particular, the RNA expression products of the RNAi expression cassette will decrease the cellular concentration of the mRNA transcript of the RNAi target gene, thus resulting in decreased concentration of the protein encoded by the RNAi target gene in the mammalian subject. Down-regulation of gene expression is specific in that a nucleotide sequence from a portion of the RNAi target gene is chosen in designing the sequence properties of the RNA coding region of the RNAi expression cassette to be transferred via rAAV-mediated gene transfer into the cells of a mammalian subject in vivo; or alternatively said: Inhibition is sequence-specific in that nucleotide sequences corresponding to the duplex region of the double-stranded RNA complex are targeted for RNA interference.

Quantization of the amount of gene expression allows one to determine a degree of inhibition which is greater than 10%, 33%, 50%, 90%, 95% or 99% as compared to a cell not treated according to the present invention. Quantization of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

In another aspect, the present invention relates to methods of controlling the expression of known genes or known nucleic acid sequences in mammalian cells in vivo by expressing sense and antisense RNA sequences (with respect to the gene or nucleic acid sequence of the RNAi target) capable of forming double-stranded RNA complexes and inducing RNAi. In that context, the RNA molecules are expressed by administering in vivo a recombinant adeno-associated viral vector comprising an RNAi expression cassette encoding said RNA molecule(s). Thus, the invention also relates to rAAV-mediated expression of RNA molecules for forming dsRNA complexes, to DNA molecules (e.g., RNAi expression cassettes) encoding the RNA molecules for forming dsRNA complexes, to rAAV vectors/virions and cells comprising such molecules, to compositions comprising said rAAV vectors/virions, and to prophylactic and therapeutic methods for administering said rAAV vectors/virions.

The invention also provides RNAi expression cassettes that encode the RNA molecule(s) capable of forming a double-stranded RNA complex and thus capable of inducing RNA interference. Such RNAi expression cassettes may be a single DNA molecule as part of a rAAV genome which, when introduced into a cell, gives rise to a single RNA molecule capable of forming intramolecularly a dsRNA complex. However it will be understood from the following description that more than one rAAV genome or RNAi expression cassette or RNA coding region may be introduced into a cell, either simultaneously or sequentially, to give rise to two or more RNA molecules capable of forming intermolecularly a dsRNA complex. Typically, the two RNA moieties capable of forming a dsRNA complex, whether intra- or intermolecularly, are at least in part sense and at least in part antisense sequences of a gene or nucleic acid sequence whose expression is to be down-regulated or decreased. The transcribed RNA strands may or may not be polyadenylated; the RNA strands may or may not be capable of being translated into a polypeptide by a cell's translational apparatus.

The design of the RNAi expression cassette does not limit the scope of the present invention. Different strategies to design an RNAi expression cassette can be applied, and RNAi expression cassettes based on different designs will be able to induce RNA interference in vivo. (Although the design of the RNAi expression cassette does not limit the scope of the invention, some RNAi expression cassette designs are included in the detailed description of this invention and below. ) The RNAi expression cassette may use a regulatory region (e.g., promoter, enhancer, silencer, splice donor and acceptor, polyadenylation) to transcribe an RNA coding region. Down-regulation of gene expression may be targeted by specific transcription in an organ, tissue, or cell type, stimulation of an environmental condition (e.g., infection, stress, temperature, chemical inducers); and/or engineering transcription at a developmental stage or age.

Features common to all RNAi expression cassettes are that they comprise an RNA coding region which encodes an RNA molecule which is capable of inducing RNA interference either alone or in combination with (an)other RNA molecule(s) by forming a double-stranded RNA complex either intramolecularly or intermolecularly.

Different design principles can be used to achieve that same goal and are known to those of skill in the art. For example, the RNAi expression cassette may encode one or more RNA molecules. After or during RNA expression from the RNAi expression cassette, a double-stranded RNA complex may be formed by either a single, self-complementary RNA molecule or two complementary RNA molecules. Formation of the dsRNA complex may be initiated either inside or outside the nucleus.

In one aspect there is provided a double-stranded RNA complex, which comprises, a first RNA portion capable of hybridizing under physiological conditions to at least a portion of an mRNA molecule, and a second RNA portion wherein at least a part of the second RNA portion is capable of hybridizing under physiological conditions to the first portion. Preferably the first and second portions are part of the same RNA molecule and are capable of hybridization at physiological conditions, such as those existing within a cell, and upon hybridization the first and second portions form a double-stranded RNA complex.

In another aspect there is provided a linear RNA molecule for forming a double-stranded RNA complex, which RNA comprises a first portion capable of hybridizing to at least a portion of an mRNA molecule, preferably within a cell and a second portion wherein at least part of the second portion is capable of hybridizing to the first portion to form a hairpin dsRNA complex.

In yet another aspect, the method comprises AAV-mediated expression of RNA with partial or fully double-stranded character in vivo.

A dsRNA complex containing a nucleotide sequence identical to a portion of the RNAi target gene is preferred for inhibition. RNA sequences with insertions, deletions, and single point mutations relative to the RNAi target sequence have also been found to be effective for inhibition. Thus, sequence identity may be optimized by alignment algorithms known in the art and calculating the percent difference between the nucleotide sequences. Alternatively, the duplex region of the dsRNA complex may be defined functionally as a nucleotide sequence that is capable of hybridizing with a portion of the RNAi target gene transcript.

In the preferred embodiment, the RNAi expression cassette comprises at least one RNA coding region. Preferably the RNA coding region is a DNA sequence that can serve as a template for the expression of a desired RNA molecule in the host cell. In one embodiment, the RNAi expression cassette comprises two or more RNA coding regions. The RNAi expression cassette also preferably comprises at least one RNA Polymerase III promoter. The RNA Polymerase III promoter is operably linked to the RNA coding region, and the RNA coding region can also be linked to a terminator sequence. In addition, more than one RNA Polymerase III promoters may be incorporated.

In certain embodiments the invention employs ribozyme-containing RNA molecules to generate dsRNA complexes, thereby overcoming certain known difficulties associated with generating dsRNA, such as for example the removal polyadenylation signals, thus preventing or minimizing release of the RNA molecule from the nucleus of a cell. In other embodiments the invention is based on the ability of a portion of the RNA molecule to encode an RNA or protein that enhances specific activity of dsRNA. One example of this specific activity-enhancing portion of the RNA molecule is a portion of the molecule encoding the HIV Tat protein to inhibit the cellular breakdown of dsRNA complexes. Such a portion is additionally useful in treating disorders such as HIV infection.

In another aspect of the present invention, the RNA expression products of the RNAi expression cassette lead to the generation of a double-stranded RNA complex for inducing RNA interference and thus down-regulating or decreasing expression of a mammalian gene. The dsRNA complex comprises a first nucleotide sequence that hybridizes under stringent conditions, including a wash step of 0.2.times.SSC at 65.degree. C., to a nucleotide sequence of at least one mammalian gene and a second nucleotide sequence which is complementary to the first nucleotide sequence. The first nucleotide sequence might be linked to the second nucleotide sequence by a third nucleotide sequence (e.g., an RNA loop) so that the first nucleotide sequence and the second nucleotide sequence are part of the same RNA molecule (scenario 1); alternatively, the first nucleotide sequence might be part of one RNA molecule and the second nucleotide sequence might be part of another RNA molecule (scenario 2). Thus, in scenario 1, the dsRNA complex is formed by intramolecular hybridization or annealing whereas in scenario 2, the ds RNA complex is formed by intermolecular hybridization or annealing.

In one embodiment, the first nucleotide sequence of said ds RNA complex is at least 17, 18, 19, 20, 21, 22, 25, 50, 100, 200, 300, 400, 500, 800 nucleotides in length.

In another embodiment, the first nucleotide sequence of said ds RNA complex is identical to at least one mammalian gene.

In another embodiment, the first nucleotide sequence of said ds RNA complex is identical to (at least) one mammalian gene.

In yet another embodiment, the first nucleotide sequence of said ds RNA complex hybridizes under stringent conditions to at least one human gene.

In still another embodiment, the first nucleotide sequence of said ds RNA complex is identical to at least one human gene.

In still another embodiment, the first nucleotide sequence of said ds RNA complex is identical to one human gene.

In one embodiment, said double-stranded RNA complex is a hairpin comprising a first nucleotide sequence that hybridizes under stringent conditions to a nucleotide sequence of at least one mammalian gene, and a second nucleotide sequence which is a complementary inverted repeat of said first nucleotide sequence and hybridizes to said first nucleotide sequence to form a hairpin structure. The first nucleotide sequence of said double-stranded RNA complex can hybridize to either coding or non-coding sequence of at least one mammalian gene.

In another aspect of the invention, expression of the RNA coding region results in the down regulation of an RNAi target gene. Preferably the target gene comprises a sequence that is at least about 90% identical with the RNA coding region, more preferably at least about 95% identical, and even more preferably at least about 99% identical.

The RNAi target gene does not limit the scope of this invention and may be any gene derived from the cell, an endogenous gene, a transgene, or a gene of a pathogen which is present in the cell after infection thereof. Thus, the choice of the RNAi target gene is not limiting for the present invention: The artisan will know how to design an RNAi expression cassette to down-regulate the gene expression of any RNAi target gene of interest. Depending on the particular target gene and the dose of rAAV virions delivered, the procedure may provide partial or complete loss of function for the target gene.

Additionally, the RNAi target cell to be transduced in vivo does not limit the scope of this invention and may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. The RNAi target cell might be a muscle cell, a liver cell, a lung cell or a brain cell. In its preferred embodiment, the RNAi target cell is a photoreceptor cell.

Moreover, the use of a specific AAV serotype does not limit the scope of this invention. Different AAV serotypes can be used to transduce different types of cells, and the tissue tropism of different AAV serotypes are known to those of skill in the art or can be determined by the artisan without undue effort. Thus, the artisan will choose the most appropriate AAV serotype for the transfer of an RNAi expression cassette into the corresponding RNAi target cell type.

According to a further aspect of the invention, the rAAV vector may also comprise a nucleotide sequence encoding a gene of interest. The gene of interest is preferably operably linked to a Polymerase II promoter. Such a construct also can contain, for example, an enhancer sequence operably linked with the Polymerase II promoter. The gene of interest is not limited in any way and includes any gene that the skilled practitioner desires to have expressed. For example, the gene of interest may be one that encodes a protein that serves as a marker to identify transduced cells. In other embodiments the gene of interest encodes a protein that has a therapeutic or palliative effect on the mammalian subject. In addition, more than one gene of interest may be included in the rAAV vector. For example a gene encoding a marker protein may be placed after the primary gene of interest to allow for identification of cells that are expressing the desired protein.

In one embodiment, the RNAi target gene is the Rhodopsin gene and the gene of interest is a version of the Rhodopsin transgene (cDNA) with silent point mutations in the RNAi target sequence so that this Rhodopsin gene version with silent point mutations will not be subject to the RNA interference induced by rAAV-mediated transfer of such an RNAi expression cassette.

In another embodiment a fluorescent marker protein, preferably green fluorescent protein (GFP), is incorporated into the construct along with the gene of interest. If a second reporter gene is included, an internal ribosomal entry site (IRES) sequence is also preferably included.

In yet another embodiment, the gene of interest is a gene included for safety concerns to allow for the selective killing of the transduced RNAi target cells within a heterogeneous population, for example within a mammal, or more particularly within a human patient. In one such embodiment, the gene of interest is a thymidine kinase gene (TK) the expression of which renders a target cell susceptible to the action of the drug gancyclovir.

Practice of the present invention will provide useful medical applications as described below under “Utility Of The Present Invention”. Moreover, this discovery of the value of rAAV-gene transfer mediated RNAi for down-regulating, decreasing or inhibiting mammalian gene expression/offers a tool for developing new strategies for blocking gene function, and for producing AAV-based RNAi vectors to treat human disease. The invention provides the method, wherein the cells are mammalian cells, and in one embodiment the cells are human. In the present invention, the double-stranded RNA complex expressed by the RNAi expression cassette transferred into the RNAi target cell via a rAAV vector can be used to inhibit an RNAi target gene which causes or is likely to cause disease, i.e. it can be used for the treatment or prevention of disease. In the prevention of disease, the RNAi target gene may be one which is required for initiation or maintenance of the disease, or which has been identified as being associated with a higher risk of contracting the disease.

Thus, the invention provides a method for treating a mammalian subject with a genetic disorder or disease caused by overexpression of a gene or by expression of a mutated gene by administering to the mammalian subject in vivo a rAAV vector comprising an RNAi expression cassette for initiating down-regulation of the RNAi target gene expression at the mRNA level, wherein the method comprises using RNAi to achieve post-transcriptional gene silencing. In this embodiment, the preferred mammalian subject is a human patient. An embodied RNAi target cell in the method of the invention is a pathogen infected cell or a tumor cell, and the tumor cell may be malignant. In another preferred embodiment, the RNAi target cell is a photoreceptor cell, and the RNAi target gene is the Rhodopsin gene. Moreover, in these embodiments, as in the method above, the method further comprises initiating RNAi, wherein the dsRNA complex is specific for the intended RNAi target gene.

Thus, the present invention may be used for the treatment or prevention of disease by administering to a mammalian subject in vivo a recombinant adeno-associated viral vector comprising an RNAi expression cassette. For example, an RNAi expression cassette may be introduced into a cancerous cell or tumor via rAAV gene transfer and—upon expression of the RNAi cassette—inhibit gene expression of a gene required for maintenance of the carcinogenic/tumorigenic phenotype. To prevent a disease or other pathology, an RNAi target gene may be selected which is required for initiation or maintenance of the disease/pathology. Treatment would include amelioration of any symptom associated with the disease or clinical indication associated with the pathology.

A gene derived from any pathogen may be targeted for inhibition. For example, the gene could cause immuno-suppression of the host directly or be essential for replication of the pathogen, transmission of the pathogen, or maintenance of the infection. For example, cells at risk for infection by a pathogen or already infected cells, particularly human immunodeficiency virus (HIV) infections, may be targeted for treatment by introduction of an RNAi expression cassette via rAAV-mediated gene transfer according to the invention. The RNAi target gene might be a pathogen or host gene responsible for entry of a pathogen into its host, drug metabolism by the pathogen or host, replication or integration of the pathogen's genome, establishment or spread of an infection in the host, or assembly of the next generation of pathogen. Methods of prophylaxis (i.e., prevention or decreased risk of infection), as well as reduction in the frequency or severity of symptoms associated with infection, can be envisioned.

The present invention could be used for treatment or development of treatments for cancers of any type, including solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelibma, myxoma, myxosarcoma, osteoma, osteosarcoma, Ewing sarcoma, synovioma, adenofibroma, adenolymphoma, carcinosarcoma, chordoma, cranio-pharyngioma, dysgerminoma, hamartoma, mesenchymoma, mesonephroma, myosarcoma, ameloblastoma, cementoma, odontoma, teratoma, thymoma, trophoblastic tumor, adenocarcinoma, carcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyl lodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neoplasms (e.g., bone, breast, digestive system, colorectal, liver, pancreatic, pituitary, testicular, orbital, head and neck, central nervous system, acoustic, pelvic, respiratory tract, and urogenital), neurofibromatosis, and cervical dysplasia, and for treatment of other conditions in which cells have become immortalized or transformed. The invention could be used in combination with other treatment modalities, such as chemotherapy, cryotherapy, hyperthernia, radiation therapy, and the like.

As disclosed herein, the present invention is not limited to any type of RNAi target gene or nucleotide sequence. The following classes of possible RNAi target genes are listed for illustrative purposes: developmental genes (e.g., adhesion molecules, cyclin kinase inhibitors, Wnt family members, Pax family members, Winged helix family members, Hox family members, cytokines/lymphokines and their receptors, growth/differentiation factors and their receptors, angiogenic factors and their receptors such as VEGF, HIF, VEGFR, antiangiogenic factors and their receptors, neurotransmitters and their receptors); oncogenes (e.g., ABL1, BCL1, BCL2, BCL6, CBFA2, CBL, CSF1R, ERBA, ERBB, EBRB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, topoisomerases, and xylanases). In one preferred embodiment, the RNAi target gene is the Rhodopsin gene, either in its non-mutated (non-pathogenic) form or its mutated (pathogenic) form.

The practice of the present invention will employ, unless otherwise indicated, conventional methods of virology, microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)It must be noted that as used herein and in the appended claims, the singular forms “a” and “the” include plural referents unless the context clearly dictates otherwise. Thus, for example, reference to “a cell” or “the cell” includes a plurality (“cells” or “the cells”), and so forth. Moreover, the word “or” can either be exclusive in nature (i.e., either A or B, but not A and B together), or inclusive in nature (A or B, including A alone, B alone, but also A and B together) unless the context clearly dictates otherwise. One of skill in the art will realize which interpretation is the most appropriate unless it is detailed by reference in the text as “either A or B” (exclusive “or”) or “and/or” (inclusive “or”).

(1) Definitions

In describing the present invention, the following terms will be employed, and are intended to be defined as indicated below.

For purposes of this invention, the term “gene therapy” means the transfer of nucleic acid compositions into cells of a multicellular eukaryotic organism, be it in vivo, ex vivo or in vitro (see also [67] [68]). The term “gene therapy” should not be limited to the purpose of correcting metabolic disorders, but be interpreted more as a technical term for the transfer of nucleic acid compositions for therapeutic purposes in general, independent of a specific therapeutic purpose. Therefore, the term “gene therapy” would include without limitation correction of metabolic disorders, cancer therapy, vaccination, monitoring of cell populations, cell expansion, stem cell manipulation etc. by means of transfer of nucleic acid compositions.

For purposes of this invention, “transfection” is used to refer to the uptake of nucleic acid compositions by a cell. A cell has been “transfected” when an exogenous nucleic acid composition has crossed the cell membrane. A number of transfection techniques are generally known in the art. See, e.g., [69, 70], Sambrook et al. (1989) Molecular Cloning, a laboratory manual, Cold Spring Harbor Laboratories, New York, Davis et al. (1986) Basic Methods in Molecular Biology, Elsevier, and [71]. Such techniques can be used to introduce one or more nucleic acid compositions, such as a plasmid vector and other nucleic acid molecules, into suitable host cells. The term refers to both stable and transient uptake of the genetic material. For purposes of this invention, “transduction” is a special form of “transfection” via a viral vector.

For purposes of this invention, “transduction” denotes the delivery of a nucleic acid composition to, into or within a recipient cell either in vivo, in vitro or ex vivo, via a virus or viral vector, such as via a recombinant AAV virion. Transduction is a special form of transfection, i.e., the term transfection includes the term transduction.

For purposes of this invention, “nucleic acid composition transfer”, “nucleic acid composition delivery”, “gene transfer” or “gene delivery” refers to methods or systems for transferring nucleic acid compositions into host cells. Such methods can result in transient expression of non-integrated transferred DNA, extrachromosomal replication and expression of transferred replicons (e.g., episomes), or integration of transferred genetic material into the genomic DNA of host cells. Nucleic acid composition transfer provides a unique approach for the treatment of inherited and acquired diseases including cancer. A number of systems and methods have been developed for nucleic acids composition transfer into mammalian cells. The transfer of an RNAi expression cassette is one example of a nucleic acid composition transfer.

For purposes of this invention, by “vector”, “transfer vector”, “gene transfer vector” or “nucleic acid composition transfer vector” is meant any element, such as a plasmid, phage, transposon, cosmid, chromosome, virus, virion, etc., which is capable of transferring and/or transporting a nucleic acid composition to a host cell, into a host cell and/or to a specific location and/or compartment within a host cell. Thus, the term includes cloning and expression vehicles, as well as viral and non-viral vectors and potentially naked or complexed DNA. However, the term does not include cells that produce gene transfer vectors such as retroviral packaging cell lines.

For purposes of this invention, by “AAV vector”, “AAV-based vector”, “adeno-associated virus based vector”, “adeno-associated viral vector”, “rAAV vector” or “recombinant adeno-associated viral vector” is meant a vector derived from an adeno-associated virus serotype, including without limitation, AAV-1, AAV-2, AAV-3, AAV-4, AAV-5, AAV-6, AAV-7, AAV-8 etc. or any other virus or serotype which is substantially homologous in its capsid protein sequence to the AAV2 or AAV5 capsid protein sequence. The term also includes hybrid vectors combining characteristics of more than one AAV serotype. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, as long as the sequences provide for functional rescue, replication and packaging.

For purposes of this invention, by “AAV vector construct” or “rAAV vector construct” is meant a nucleic acid composition that is used in the production of rAAV virions (rAAV vectors). More specifically, rAAV vector constructs give rise to the rAAV genomes to be packaged into the rAAV capsids. AAV vector constructs are constructed using known techniques to at least provide, as operatively linked components in the direction of transcription, (a) control elements including a transcriptional initiation region, (b) the DNA of interest (here: at least one RNAi expression cassette), and (c) a transcriptional termination region. The resulting construct which contains the operatively linked components is bounded (5′ and 3′) with functional AAV ITR sequences. An example of an AAV vector construct can be a double-stranded DNA plasmid.

For purposes of this invention, by “recombinant virus”, “recombinant virion”, “recombinant vector” or “recombinant viral vector” is meant a virus that has been genetically altered, e.g., by the addition or insertion of a heterologous nucleic acid composition into the particle.

For purposes of this invention, by “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion, and both strands are equally infectious.

For purposes of this invention, a “recombinant AAV virion” or “rAAV virion” is defined herein as an infectious, replication-defective virus composed of an AAV protein shell, encapsidating a heterologous DNA molecule of interest which is flanked on one or both sides by AAV ITRs. A rAAV virion is produced in a suitable host cell which has had an AAV vector construct, AAV helper functions and accessory functions introduced therein. In this manner, the host cell is rendered capable of encoding AAV polypeptides that are required for packaging the AAV vector (comprising a recombinant nucleotide sequence of interest) into recombinant virion particles for subsequent gene delivery. The term “rAAV virion” and its synonyms and the term “rAAV vector” and its synonyms can be used interchangeably unless the context clearly dictates otherwise.

For purposes of this invention, “pseudotyped” (r)AAV refers to a recombinant AAV in which the capsid protein is of a serotype heterologous to the serotype(s) of the ITRs of the minigene. For example, a pseudotyped rAAV may be composed of a minigene carrying AAV5 ITRs and cap sid of AAV2, AAV1, AAV3, AAV4, AAV6, AAV7, AAV8 or another suitable AAV serotype, where the minigene is packaged in the heterologous capsid. Alternatively, a pseudotyped rAAV may be composed of an AAV5 capsid which has packaged therein a minigene containing ITRs from at least one of the other serotypes. Particularly desirable rAAV composed of AAV5 are described in U.S. patent application Ser. No. 60/200,409, filed Apr. 28, 2000 and International Patent Application No. PCT/USO1/13000, filed Apr. 23, 2001, both of which are incorporated by reference herein.

As defined herein, AAV capsid proteins include hybrid capsid proteins which contain a functional portion of one or more AAV capsid proteins. Such hybrid capsid proteins may be constructed such that a fragment of a capsid derived from one serotype is fused to a fragment of a capsid from another serotype to form a single hybrid capsid which is useful for packaging of an AAV minigene.

For purposes of this invention, the term “protein” means a polypeptide (native (i.e., naturally-occurring) or mutant), oligopeptide, peptide, or other amino acid sequence. As used herein, “protein” is not limited to native or full-length proteins, but is meant to encompass protein fragments having a desired activity or other desirable biological characteristics, as well as mutants or derivatives of such proteins or protein fragments that retain a desired activity or other biological characteristic. Mutant proteins encompass proteins having an amino acid sequence that is altered relative to the native protein from which it is derived, where the alterations can include amino acid substitutions (conservative or non-conservative), deletions, or additions (e.g., as in a fusion protein). “Protein” and “polypeptide” are used interchangeably herein without intending to limit the scope of either term.

For purpose of this invention, “desired protein” refers to proteins encoded by minicassettes or minigenes used in the present invention, which either act as target proteins for an immune response, or as a therapeutic or compensating protein in gene therapy regimens.

For purposes of this invention, by “DNA” is meant a polymeric form of desoxyribonucleotides (adenine [A], guanine [G], thymine [T], or cytosine [C]) in double-stranded or single-stranded form, either relaxed and supercoiled, either linear or circular. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the non-transcribed strand of DNA (i.e., the strand having the sequence homologous to the mRNA). The term captures molecules that include the four bases adenine, guanine, thymine, or cytosine, as well as molecules that include base analogues which are known in the art.

For purposes of this invention, the term “polynucleotide” as used herein means a polymeric form of nucleotides of any length, either ribonucleotides or desoxyribonucleotides. This term refers only to the primary structure of the molecule. Thus, the term includes double- and single-stranded DNA, as well as, double- and single-stranded RNA as well as DNA/RNA hybrids. It also includes modifications, such as methylation or capping, and unmodified forms of the polynucleotide. A polynucleotide can be delivered to a cell to express a protein, or to express an exogenous nucleotide sequence, to inhibit, eliminate, augment, or alter expression of an endogenous nucleotide sequence, or to express a specific physiological characteristic not naturally associated with the cell. Polynucleotides may be anti-sense.

For purposes of this invention, the term “nucleic acid composition” means any nucleic acid molecule, may it be single stranded, double stranded or triple helical or a mixture thereof, may it be DNA, RNA, PNA, a DNA/RNA hybrid (e.g., a chimeraplast), may it be linear or circular, chemically modified, coupled to other macromolecules (e.g. proteins) or a mixture thereof, independent of its size (single nucleotide, oligonucleotide, polynucleotide). It may be in the form of a plasmid, cosmid, bacteriophage-based genome (e.g., M13-based vectors), viral vector such as a (recombinant) adenoviral genome, adeno-associated viral genome, retroviral genome, lentiviral genome, herpes virus genome, bacterial artificial chromosome, yeast artificial chromosome, mammalian artificial chromosome, or any part or parts or combinations thereof. The nucleic acid composition comprises a nucleotide sequence that encodes a desired protein or peptide, serves as a template for functional nucleic acid molecules and/or functions as a functional unit in itself such as without limitation a ribozyme, an antisense molecule, an aptamer or a short interfering RNA (siRNA). The desired protein/peptide and/or functional nucleic acid molecule may be any product of medical, industrial or scientific interest. In many instances, the nucleic acid composition functions as a “transgene”.

In the context of this invention, the nucleic acid composition (functionally defined as an RNAi expression cassette) by means of its encoded product(s) leads either directly or indirectly to the down-regulation or decrease of the expression of an RNAi target gene via RNA interference. In one specific embodiment, the RNAi expression cassette encodes one nucleic acid composition which is an RNA molecule capable of forming intramolecularly a double-stranded RNA complex that is capable of inducing RNA interference. Said RNA molecule may or may not be a siRNA. In another specific embodiment, the RNAi expression cassette encodes two nucleic acid compositions, which are complementary RNA molecules capable of forming intermolecularly a double-stranded RNA complex that is capable of inducing RNA interference. In yet another embodiment, two rAAV vectors are used each comprising its own RNAi expression cassette and each expression cassette encoding one RNA molecule which the two RNA molecules being complementary to each other and capable of forming intermolecularly a double-stranded RNA complex that is capable of inducing RNA interference. One of skill in the art can generate any configuration of nucleic acid compositions and regulation mechanisms which can be used via the methods described herein to achieve the formation of an RNAi inducing dsRNA complex (Sambrook 1989, Lodish et al. 2000).

For the purpose of describing the relative position of nucleotide sequences in a particular nucleic acid molecule throughout the present invention, such as when a particular nucleotide sequence is described as being situated “upstream,” “downstream,” “5′,” or “3′” relative to another sequence, it is to be understood that it is the position of the sequences in the non-transcribed strand of a DNA molecule that is being referred to as is conventional in the art.

Natural “nucleic acids” have a phosphate backbone, artificial nucleic acids may contain other types of backbones. Nucleotides are the monomeric units of nucleic acid polymers. Sometimes, the term “base” is used interchangeably with “nucleotide” specifically in the context of polynucleotides and more specifically, in the context of double-stranded polynucleotides where the term “base pair” refers to two complementary, paired nucleotides within the double-stranded molecule. (For example, a double-ganglioma, stranded DNA molecule might comprise 20 nucleotides or bases, organized as 10 base pairs. ) The term “nucleic acid” includes de(s)oxyribonucleic acid (DNA) and ribonucleic acid (RNA). RNA may be in the form of a tRNA (transfer RNA), snRNA (small nuclear RNA), rRNA (ribosomal RNA), mRNA (messenger RNA), anti-sense RNA, siRNA, and ribozymes. The term “nucleic acid” also includes PNAs (peptide nucleic acids), phosphorothioates, and other variants of the phosphate backbone of native nucleic acids.

For purposes of this invention, a “gene sequence”, “coding sequence”, “coding region”, “open reading frame” or a sequence which “encodes” a particular RNA or protein, is a nucleic acid composition which is transcribed into RNA (in the case of DNA) and potentially translated (in the case of mRNA) into a polypeptide in vitro or in vivo when placed under the control of appropriate regulatory sequences. More specifically, a “protein coding region” or “protein coding sequence” encodes a protein, whereas an “RNA coding region” or “RNA coding sequence” encodes an RNA molecule. Said RNA molecule might possess a specific function either alone or in combination with other RNA molecules and/or proteins, such as—for example—inducing RNA interference or functioning as a ribozyme. Said RNA molecule might also be additionally translated into a protein. Thus, an “RNA coding region” or “RNA coding sequence” is a nucleic acid composition that can serve as a template for the synthesis of an RNA molecule, such as an siRNA. Preferably, the RNA coding region is a DNA sequence.

The boundaries of a gene encoding a protein are determined by a start codon corresponding to the 5′ (amino) terminus of the protein and potentially a translation stop codon corresponding to the 3′ (carboxy) terminus.

A gene sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

For purposes of this invention, by the term “transgene” is meant a nucleic acid composition made out of DNA, which encodes a peptide, oligopeptide or protein. The transgene may be operatively linked to regulatory components in a manner which permits transgene transcription, translation and/or ultimately directs expression of a product encoded by the nucleic acid composition in the host cell, e.g., the transgene is placed into operative association with a promoter and enhancer elements, as well as other regulatory sequences, such as introns or polyA sequences, useful for its regulation. The composite association of the transgene with its regulatory sequences is referred to herein as a “minicassette” or “minigene”. Minicasssettes or minigenes in their entirety are also nucleic acid compositions. The exact nucleic acid composition will depend upon the use to which the resulting nucleic acid transfer vector will be put and is known to the artisan (Sambrook 1989, Lodish et al. 2000). When taken up by a target cell, the nucleic acid composition may remain present in the cell as a functioning extrachromosomal molecule, or it may integrate into the cell's chromosomal DNA, depending on the kind of transfer vector used.

For purposes of this invention, “AAV minigene” refers to a construct composed of, at a minimum, AAV ITRs and a heterologous nucleic acid composition. For production of rAAV according to the invention, a minigene may be carried on any suitable vector, including viral vectors, plasmid vectors, and the like.

“RNA transcript” refers to the product resulting from RNA polymerase-catalyzed transcription of a DNA sequence. When the RNA transcript is a perfect complementary copy of the DNA sequence, it is referred to as the primary transcript; when the RNA sequence is derived from post-t ranscriptional processing of the primary transcript, it is referred to as the mature RNA. “Messenger RNA (mRNA)” refers to the RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded form using the Klenow fragment of DNA polymerase I. “Sense” RNA refers to an RNA transcript that includes the mRNA and can be translated into protein within a cell or in vitro. “Antisense RNA” refers to an RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, siRNA, or other RNA that may not be translated but yet has an effect on cellular processes. The terms “complement” and “reverse complement” are used interchangeably herein with respect to mRNA transcripts, and are meant to define the antisense RNA of the message.

For purposes of this invention, “heterologous” as it relates to nucleic acid compositions denotes sequences that are not normally joined together. Thus, a “heterologous” region of a nucleic acid composition is a segment of nucleic acid within or attached to another nucleic acid composition that is not found in association with the other molecule in nature. For example, a heterologous region of a nucleic acid composition could include a coding sequence flanked by sequences not found in association with the coding sequence in nature. Another example of a heterologous coding sequence is a construct where the coding sequence itself is not found in nature (e.g., synthetic sequences having codons different from the native gene). Allelic variation or naturally occurring mutational events do not give rise to heterologous DNA, as used herein.

For purposes of this invention, the term “control elements” or “regulatory sequences” refers collectively to promoter regions, polyadenylation signals, transcription termination (terminator) sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present as long as the selected coding sequence is capable of being replicated, transcribed and/or translated in an appropriate host cell. Sometimes, the entirety of control elements and coding sequence is referred to as “gene”; in other instances, “gene” only refers to the coding sequence. For purposes of this invention, “gene” refers to the entirety of control elements and coding sequence. Expression control elements include appropriate transcription initiation, termination, promoter and enhancer sequences, efficient RNA processing signals such as splicing and transcription termination signals (e.g., polyadenylation signal for RNA Polymerase II, 5 T residues for RNA Polymerase III), sequences that stabilize cytoplasmic mRNA or RNA in general, sequences that enhance translation efficacy (i.e., Kozak consensus sequence), sequences that enhance protein stability, and when desired, sequences that enhance protein processing and/or secretion. A great number of expression control elements, e.g., native, constitutive, inducible and/or tissue specific, are known in the art and may be utilized to drive expression of the gene, depending upon the type of expression desired.

For expression of proteins in eukaryotic cells, expression control elements typically include a promoter, an enhancer, such as one derived from an immunoglobulin gene, SV40, cytomegalovirus, etc., a polyadenylation sequence, and may include splice donor and acceptor sites. The polyadenylation sequence generally is inserted following the transgene sequences and before the 3′ ITR sequence in rAAV vectors. The regulatory sequences useful in the constructs of the present invention may also contain an intron, desirably located between the promoter/enhancer sequence and the gene. One possible intron sequence is derived from SV40, and is referred to as the SV40 T intron sequence. Another suitable regulatory sequence includes the woodchuck hepatitis virus post-transcriptional element [72]. Still other methods may involve the use of a second internal promoter, an alternative splice signal, a co- or post-translational proteolytic cleavage strategy, among others which are known to those of skill in the art. Selection of these and other common vector and regulatory sequences are conventional, and many such sequences are available. See, e.g., Sambrook et al, and references cited therein at, for example, pages 3.18-3.26 and 16.17-16.27 and Ausubel et al., CURRENT PROTOCOLS IN MOLECULAR BIOLOGY, John Wiley & Sons, New York, 1989.

One of skill in the art may make a selection among these regulatory sequences without departing from the scope of this invention. Suitable promoter/enhancer sequences may be selected by one of skill in the art using the guidance provided by this application. Such selection is a routine matter and is not a limitation of the present invention. For instance, one may select one or more regulatory sequences operably linked to the RNA coding region of the RNAi expression cassette for insertion in a “AAV mini-gene” which is composed of the 5′ ITRs, the RNAi expression cassette, and 3′ ITRs in the context of rAAV vectors. Thus, this system permits a great deal of latitude in the selection of the various components of the minigene. Provided with the teachings of this invention, the design of such a minigene can be made by resort to conventional techniques.

For purposes of this invention, the term “promoter” means a regulatory sequence capable of binding RNA polymerase and/or a regulatory sequence sufficient to direct transcription. “Promoter” is also meant to encompass those promoter (or enhancer) elements for cell-type specific, tissue-specific and/or inducible (by external signals or agents) transcription; such elements may be located in the 5′ or 3′ regions of a native gene. A promoter might bind RNA Polymerase I, RNA Polymerase II and/or RNA Polymerase III.

In some embodiments, RNA Polymerase III—based promoters are desired as part of the RNAi expression cassette. RNA Polymerase III promoters are well known to one of skill in the art. A suitable range of RNA Polymerase III promoters can be found, for example, in Paule and White. Nucleic Acids Research., Vol 28, pp 1283-1298 (2000), which is hereby incorporated by reference in its entirety. The definition of RNA Polymerase III promoters also include any synthetic or engineered DNA fragment that can direct RNA Polymerase III to transcribe a downstream RNA coding sequence. Further, the RNA Polymerase III (Pol III) promoter or promoters used as part of the rAAV vector can be inducible. Any suitable inducible Pol III promoter can be used with the methods of the invention. Particularly suited Pol III promoters include the tetracycline responsive promoters provided in Ohkawa and Taira Human Gene Therapy, Vol. 11, pp 577-585 (2000) and in Meissner et al. Nucleic Acids Research, Vol. 29, pp 1672-1682 (2001), which are incorporated herein by reference. Examples of RNA Polymerase III promoters include—but are not limited to—the U6[42] and H1 [73] promoters. One key advantage of using a Pol III system is that transcription terminates at a defined stretch of thymidine residues, leaving one to four uridines at the 3′-terminus of the nascent RNA, thereby making it similar to many siRNAs.

In yet other embodiments, the RNAi expression cassette comprises a RNA Polymerase I (RNA Pol I) or RNA Polymerase III (RNA Pol II) promoter. The inventors are the first to achieve RNA interference in the context of RNA Polymerase I driven RNA expression.

In other embodiments, high-level constitutive expression of a protein-coding gene of interest is desired. Examples of promoters useful for that purpose include, without limitation, the retroviral Rous sarcoma virus (RSV) LTR promoter (optionally with the RSV enhancer), the cytomegalovirus (CMV) promoter (optionally with the CMV enhancer) [74], the SV40 promoter, the dihydrofolate reductase promoter, the .beta.-actin promoter, the phosphoglycerol kinase (PGK) promoter, and the EF1.alpha. promoter (Invitrogen).

Inducible promoters are regulated by exogenously supplied compounds, including, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter [75], the tetracycline-repressible system [76], the tetracycline-inducible system [77]; see also U.S. patent application: 0030013189, [78], the RU486-inducible system [79, 80] and the rapamycin-inducible system [81]. Other types of inducible promoters which may be useful in the transgenes and other constructs described herein are those which are regulated by a specific physiological state, e.g., temperature, acute phase, a particular differentiation state of the cell, or in replicating cells only.

For purposes of this invention, the term “operative association” or “operative linkage” refers to an arrangement of elements or nucleic acid sequences wherein the components so described are configured so as to perform their intended function. Thus, (a) regulatory sequence(s) operably linked to a coding sequence are capable of effecting the expression of said coding sequence and are connected in such a way as to permit expression of the coding sequence when the appropriate molecules (e.g., transcriptional activator proteins) are bound to the regulatory sequence(s). The regulatory sequences need not be contiguous with the coding sequence, as long as they function to direct the expression thereof. Thus, for example, intervening untranslated yet transcribed sequences can be present between a promoter sequence and the coding sequence and the promoter sequence can still be considered “operably linked” to the coding sequence. “Operably linked” sequences include both expression control sequences that are contiguous with the coding sequences for the product of interest and expression control sequences that act in trans or at a distance to control the expression of the product of interest.

For purposes of this invention, “homology” or “homologous” refers to the percent homology between two polynucleotide or between two polypeptide moieties. The correspondence between the sequence from one moiety to another can be determined by techniques known in the art. Two polynucleotide or two polypeptide sequences are “substantially homologous” to each other when at least about 80%, preferably at least about 90%, and most preferably at least about 95% of the nucleotides or amino acids match over a defined length of the molecules, as determined using methods in the art.

The techniques for determining amino acid sequence homology are well known in the art. In general, “homology” means the exact amino acid to amino acid comparison of two or more polypeptides at the appropriate place, where amino acids are identical or possess similar chemical and/or physical properties such as charge or hydrophobicity. A so-termed “percent homology” then can be determined between the compared polypeptide sequences. The programs available in the Wisconsin Sequence Analysis Package (available from Genetics Computer Group, Madison, Wis.), for example, the GAP program, are capable of calculating homologies between two polypeptide sequences. Other programs for determining homology between polypeptide sequences are known in the art.

Homology for polynucleotides is determined essentially as follows: Two polynucleotides are considered to be “substantially homologous” to each other when at least about 80%, preferably at least about 90%, and most preferably at least about 95% of the nucleotides match over a defined length of the molecules, when aligned using the default parameters of the search algorithm BLAST 2.0. The BLAST 2.0 program is publicly available.

Alternatively, homology for polynucleotides can be determined by hybridization experiments. As used herein, a first nucleic acid sequence or fragment (such as for example, primers or probes), is considered to selectively hybridize to a second nucleic acid sequence, thus indicating “substantial homology”, if such a second sequence is capable of specifically hybridizing to the first sequence or a variant or capable of specifically priming a polymerase chain reaction: (i) under typical hybridization and wash conditions, such as those described, for example, in Maniatis, (Molecular Cloning: A Laboratory Manual, 2^{nd} Edition, 1989) where preferred hybridization conditions are those of lesser stringency and more preferred, higher stringency; or (ii) using stringent wash conditions that allow at most about 25-30% basepair mismatches, for example, 2.times.SSC, 0.1% SDS, at room temperature twice, for 30 minutes each; then 2.times.SSC, 0.1% SDS, 37 C, once for 30 minutes; the 2.times.SSC at room temperature twice, 10 minutes each or (iii) under standard PCR conditions or under “touch-down” PCR conditions such as described by [82]).

The term “hybridizing conditions” as used herein shall mean conditions permitting hybridization between two complementary strands of nucleic acid in general, and between two complementary strands of RNA having a length of at least seven nucleotides in particular. Hybridizing conditions are well known in the art, and include, without limitation, physiological conditions, such as, but not limited to, intracellular physiological conditions.

For purposes of this invention, “complementarity” or “complementary” refers to the percent complementarity between two polynucleotide moieties. The complementarity between the sequence from one moiety to another can be determined by techniques known in the art. Two DNA sequences, two RNA sequences, or one DNA and one RNA sequence are “substantially complementary” to each other when at least about 80%, preferably at least about 90%, and most preferably at least about 95% of the nucleotides are complementary matches over a defined length of the molecules, as determined using methods in the art, such as e.g., the search algorithm BLAST 2.0. The BLAST 2.0 program is publicly available. Thus, more specifically, “substantial complementarity” and “substantially complementary” as used herein indicate that two nucleic acids are at least 80% complementary, more preferably at least 90% complementary and most preferably at least 95% complementary over a region of more than about 15 nucleotides and more preferably more than about 19 nucleotides.

“Complementary match” means that (1)an adenine (A) residue in one moiety is paired with a thymidine (T) or uracil (U) residue in the other moiety, (2)a cytosine (C) residue in one moiety is paired with a guanine (G) residue in the other moiety(3)a thymidine (T) or uracil (U) residue in one moiety is paired with an adenine (A) residue in the other moiety, (4)a guanine (G) residue in one moiety is paired with a cytosine (C) residue in the other moiety.

Alternatively, complementarity for polynucleotide moieties can be determined by hybridization experiments. As used herein, a nucleic acid sequence is considered to selectively hybridize to another nucleic acid sequence, thus indicating “substantial complementarity”, if such a sequence is capable of (a) specifically hybridizing to the other sequence or a variant thereof or (b) specifically priming a polymerase chain reaction: (i) under typical hybridization and wash conditions, such as those described, for example, in Maniatis, (Molecular Cloning: A Laboratory Manual, 2^{nd} Edition, 1989) where preferred hybridization conditions are those of lesser stringency and more preferred, higher stringency; or (ii) using “stringent conditions” defined as preferentially, for example, 2.times.SSC, 0.1% SDS, at room temperature twice, for 30 minutes each; then 2.times.SSC, 0.1% SDS, 37 C, once for 30 minutes; the 2.times.SSC at room temperature twice, 10 minutes each or (iii) under standard PCR conditions or under “touch-down” PCR conditions such as described by [82]), or more preferentially, defined as 0.2.times SSC at 65.degree.C. instead of 2.times SSC and 37.degree.C., respectively.

For purposes of this invention, the term “cell” means any prokaryotic or eukaryotic cell, either ex vivo, in vitro or in vivo, either separate (in suspension) or as part of a higher structure such as but not limited to organs or tissues. A cell may be germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. Cells may be somatic, undifferentiated, dedifferentiated, neoplastic, chimera cells or transgenic animal cells. The cells may be of entodermal, ectodermal or neurodermal origin.

For purposes of this invention, “lung cells” may refer to one or more of the following types of cells (without limitation): type I pneumocytes, type II pneumocytes, pseudostratified columnar epithelial cells, stratified squamous epithelial cells, gland cells, duct cells, subepithelial connective tissue cells, goblet cells, mucosal cells, submucosal cells, hyaline cartilage cells, perichondrial cells, ciliated columnar cells, basal epithelial cells, brush cells, bronchial epithelial cells, submucosal gland cells, pseudostratified ciliated columnar epithelial cells, lung tissue cells, bronchial respiratory epithelial cells, cuboid epithelial cells of brionchioles, bronchiolar epithelial cells, alveolar cells, squamous (type I) alveolar cells, great (type II) alveolar cells, and alveolar macrophages.

For purposes of this invention, the term “host cell” means a cell that can be transduced and/or transfected by an appropriate gene transfer vector. The nature of the host cell may vary from gene transfer vector to gene transfer vector. In more specific contexts, “host cell” refers to a cell that allows for production of recombinant viral vectors. In one specific embodiment of this invention, the host cell is the human embryonic kidney (HEK) cell line 293 for the production of rAAV virions. In that specific context, the term “packaging cell” or “packaging cell line” is used as a synonym for “host cell”.

For purposes of this invention, “treatment” refers to prophylaxis and/or therapy.

“Pharmaceutically effective” levels are levels sufficient to achieve a physiologic effect in a human or veterinary subject, which effect may be therapeutic or prophylactic.

For purposes of this invention, by “mammalian subject” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, hamsters, rabbits and guinea pigs, and the like. The term does not denote a particular age or sex. Thus, adult and newborn subjects, as well as fetuses, whether male or female, are intended to be covered.

For purposes of this invention, the terms “individual” or “subject” or “patient” as used herein refer to vertebrates, particularly members of the mammalian species and include but are not limited to domestic animals, sports animals, primates and humans; more particularly the term refer to humans.

The term “decrease of gene expression”, “inhibition of gene expression” or “down-regulation of gene expression” refers to the (observable) decrease in the level of protein and/or mRNA product from a target gene. Inhibition of the expression of a target gene can be verified by observing or detecting an absence or observable decrease in the level of protein encoded by a target gene (this may be detected by for example a specific antibody or other techniques known to the skilled person) and/or mRNA product from a target gene (this may be detected by for example hybridization studies) and/or phenotype associated with expression of the gene. In the context of a medical treatment, verification of inhibition of the expression of a target gene may be by observing a change in the disease condition of a subject, such as a reduction in symptoms, remission, a change in the disease state and so on. Preferably, the inhibition is specific, i.e. the expression of the target gene is inhibited without manifest effects on the other genes of the cell.

For purposes of this invention, the term “RNA interference” or “RNAi” is broadly defined and includes all post-transcriptional and transcriptional mechanisms of RNA mediated inhibition of gene expression, such as those described in P. D. Zamore Science 296, 1265 (2002). RNA interference is mediated by double-stranded RNA (dsRNA), which can induce many different epigenetic gene-silencing processes in eukaryotes, including the degradation of homologous mRNAs—a process called RNA interference (RNAi) in animals and post-transcriptional gene silencing (PTGS) in plants. RNA interference (RNAi) has first been discovered in 1998 by Andrew Fire and Craig Mello in C. elegans, confirming former studies of PGTS in plants [21]. It now seems to be a ubiquitous mechanism—also applicable to humans [6, 7, 17, 22-29]. Double stranded RNA has been shown to inhibit gene expression of genes having a complementary sequence through a process termed RNA interference (see, for example, Hammond et al. Nat. Rev. Genet. 2:110-119 (2001)). According to the invention, a ds RNA complex corresponding to a region of a gene to be down-regulated is expressed in the cell via rAAV-mediated RNAi expression cassette transfer.

For purposes of this invention, the term “small interfering RNA” or “siRNA” as used herein means short interfering RNA which is a double-stranded RNA complex that is less than 30 base pairs (i.e., 60 nucleotides or bases) and preferably 21-25 base pairs (i.e., 42 50 bases or nucleotides) in length. More generally, double-stranded RNA that is responsible for inducing RNAi is termed interfering RNA. Thus, a “small interfering RNA” or “siRNA” is a double-stranded RNA complex that is capable of decreasing the expression of a gene with which it shares homology. The region of the gene or other nucleotide sequence over which there is homology is known as the “RNAi target region”, “target region”, “RNAi target sequence” or “target sequence”.

In one embodiment the siRNA may be a “hairpin” or stem-loop RNA molecule, comprising a sense region, a loop region and an antisense region complementary to the sense region and thus capable of forming an RNAi inducing dsRNA complex. In other embodiments the siRNA comprises two distinct RNA molecules that are non-covalently associated to form a dsRNA complex.

For purposes of this invention, the term “RNAi expression cassette” as used herein means a nucleic acid composition which encodes one or more RNA molecules which are capable of forming a double-stranded RNA complex and thus are capable of inducing RNA interference. In the context of the present invention, said RNAi expression cassette(s) are part of a rAAV vector or rAAV genome.

The design of the RNAi expression cassette does not limit the scope of the invention. Different strategies to design an RNAi expression cassette can be applied, and RNAi expression cassettes based on different designs will be able to induce RNA interference in vivo. (Although the design of the RNAi expression cassette does not limit the scope of the invention, some RNAi expression cassette designs are included in the detailed description of this invention and below. ) One of skill in the art will be able to choose among different designs without undue effort.

Features common to all RNAi expression cassettes are that they comprise an RNA coding region which encodes one or more RNA molecules. After or during RNA expression from the RNAi expression cassette, a double-stranded RNA complex may be formed by either a single, self-complementary RNA molecule (intramolecular formation) or two complementary RNA molecules (intermolecular formation). Formation of the dsRNA complex may be initiated either inside or outside the nucleus. The dsRNA complex will be capable of inducing RNA interference either directly or indirectly.

In some embodiments, the RNAi inducing double-stranded RNA complex (encoded by the RNAi expression cassette(s)) comprises a first RNA portion capable of hybridizing under physiological conditions to at least a portion of an mRNA molecule (the RNAi target sequence of the RNAi target mRNA of the RNAi target gene), and a second RNA portion wherein at least a part of the second RNA portion is capable of hybridizing under physiological conditions to the first portion. Preferably the first and second portions are part of the same RNA molecule and are capable of hybridization at physiological conditions, such as those existing within a cell and upon hybridization the first and second portions form a double-stranded RNA complex. For example, the RNAi inducing double-stranded RNA complex (encoded by the RNAi expression cassette(s)) is formed by a linear RNA molecule, which RNA comprises a first portion capable of hybridizing to at least a portion of an mRNA molecule and a second portion wherein at least part of the second portion is capable of hybridizing to the first portion to form a hairpin dsRNA complex. Thus, in some embodiments, when introduced into a cell via rAAV gene transfer, expression of the RNAi expression cassette gives rise to a single RNA molecule capable of forming intramolecularly an RNAi inducing dsRNA complex. However it will be understood from the following description that more than one rAAV genome or rAAV vector or RNAi expression cassette or RNA coding region may be introduced into a cell, either simultaneously or sequentially via rAAV mediated gene transfer, to give rise to two or more RNA molecules capable of forming intermolecularly an RNAi-inducing dsRNA complex. Typically, the two RNA sequences capable of forming a dsRNA complex, whether intra- or intermolecularly, are at least in part sense and at least in part antisense sequences of a gene or nucleic acid sequence whose expression is to be down-regulated or decreased.

In the preferred embodiment the RNAi expression cassette comprises at least one RNA coding region. In other embodiments, the RNAi expression cassette comprises two or more RNA coding regions. The RNAi expression cassette also preferably comprises at least one RNA Polymerase III promoter. The RNA Polymerase III promoter is operably linked to the RNA coding region, and the RNA coding region can also be linked to a termination sequence (terminator). In addition, more than one RNA Polymerase III promoters may be incorporated.

In certain embodiments the invention employs ribozyme-containing RNA molecules—encoded by the RNAi expression cassette—to generate dsRNA complexes, thereby overcoming certain known difficulties associated with generating dsRNA such as the removal of polyadenylation signals. In other embodiments the invention is based on the ability of a portion of the RNA molecule to encode an RNA or protein that enhances specific activity of dsRNA. One example of this specific activity enhancing portion of the RNA molecule is a portion of the molecule encoding the HIV Tat protein to inhibit the cellular breakdown of dsRNA complexes. Such a portion is additionally useful in treating disorders such as HIV infection.

For purposes of this invention, the term “RNA expression product” or “RNA product” refers to the RNA molecule or RNA transcript transcribed (synthesized) from an RNAi expression cassette.

For purposes of this invention the term “target gene” or “RNAi target gene” means a targeted nucleic acid composition, the expression of which is being decreased (down-regulated) by RNA interference induced through rAAV-mediated RNAi expression cassette transfer. The dsRNA complex—encoded by the RNAi expression cassette(s)—comprises a nucleotide sequence that hybridizes under physiologic conditions of the cell to the nucleotide sequence of at least a portion of the RNAi target gene. Examples of RNAi target genes are cellular genes present in the genome or viral and pro-viral genes. The RNAi target gene may be a protein-coding gene or a non-protein coding gene, such as a gene which codes for ribosmal RNAs, splicosomal RNA, tRNAs, etc. Preferred target genes include, but are not limited to viral genes and foreign genes which have been introduced into the cell, tissue or organ or alternatively, genes which are endogenous to the cell, tissue or organ. Wherein the RNAi target gene is a viral gene, it is particularly preferred that the viral gene encodes a function which is essential for replication or reproduction of the virus, such as but not limited to a DNA polymerase or RNA polymerase gene or a viral coat protein gene, amongst others. In one preferred embodiment, the RNAi target gene is the Rhodopsin gene. In another preferred embodiment, the RNAi target sequence is a portion of the Rhodopsin gene comprising a point mutation which leads to an autosomal-dominant disease phenotype.

Further examples of RNAi target genes, without limitation, are genes related to autosomal-dominant disorders (such as autosomal dominant Retinitis Pigmentosa), genetic disorders with a dominant negative phenotype (such as autosomal dominant Retinitis Pigmentosa), cancer, rheumatoid arthritis and viruses. Cancer-related genes include oncogenes (e.g., K-ras, c-myc, bcr/abl, c-myb, c-fms, c-fos and cerb-B), growth factor genes (e.g., genes encoding epidermal growth factor and its receptor, and fibroblast growth factor-binding protein), matrix metalloproteinase genes (e.g., the gene encoding MMP-9), adhesion-molecule genes (e.g., the gene encoding VLA-6 integrin), and tumor suppressor genes (e.g., bc/-2 and bcl-XI). Rheumatoid arthritis-related genes include, for example, genes encoding stromelysin and tumor necrosis factor. Viral genes include human papilloma virus genes (related, for example, to cervical cancer), hepatitis B and C genes, and cytomegalovirus genes (related, for example, to retinitis). In one embodiment of the instant method, the cell is HIV-infected and the gene is an HIV gene. HIV genes include, without limitation, tat, nef, rev, ma, ca, nc, p.sup.6, vpu, pr, vif, su, tm, vpr, rt and in. In the preferred embodiment, the HIV gene is tat.

The following classes of possible RNAi target genes are examples of the genes which the present invention may use to down-regulate: developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABLI, BCL1, BCL2, BCL6, CBFA2, CBL, CSFIR, ERBA, ERBB, EBRB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3 and YES); tumor suppresser genes (e.g., APC, BRCA1, BRCA2, MADH4, MCC, NF1, NF2, RB1, TP53 and WT1); and enzymes (e.g., ATPases, alcohol dehydrogenases, amylases, catalases, cyclooxygenases, decarboxylases, dextrinases, DNA and RNA polymerases, galactosidases, GTP ases, tyrosine kinases, helicases, integrases, invertases, isomerases, kinases, lactases, lipases, lipoxygenases, lysozymes, peroxidases, phosphatases, phospholipases, phosphorylases, proteinases and peptideases, recombinases, reverse transcriptases, topoisomerases), and receptors (G-protein coupled receptors, viral receptors, receptors with tyrosine kinase activity, Rhodopsin, insulin receptor, receptors for growth factors, receptors for second messengers, receptors for small molecules, receptors with protein ligands, VEGF receptor, HIF receptor, EGFR receptor).

For purposes of this invention, the “RNAi target cell” is the cell from which the RNAi target gene is expressed, and in which the gene expression is disrupted by RNAi, wherein exposure to the dsRNA complex homologous to the RNAi target gene initiates the disruption. The disruption is detected and measurable in terms of “inhibition” or reduction of the expression of the RNAi target gene, which is reflected in terms of a reduction or decrease of activity of the expression product, as compared with the activity, absent treatment with the homologous dsRNA, from the targeted gene.

The term “target mRNA” or “RNAi target mRNA” refers to any mRNA whose expression in the host is to be reduced. The RNAi target mRNA is the RNA transcript of the (RNAi) target gene.

The terms “double-stranded RNA complex” or “dsRNA complex” as used herein are equivalent, and each shall mean a complex formed either (a) by two linear molecules of RNA, wherein at least a portion of the sequence of one molecule is complementary to, and is capable of or has hybridized to, at least a portion of the sequence of the other RNA molecule, or (b) by two portions of a linear RNA molecule which are complementary to, and are capable of or have therefore hybridized to, each other. The dsRNA complex is generated by the RNA expression product(s) of the RNAi expression cassette(s) and is able to mediate either directly or indirectly RNA interference, thus mediating down-regulation of the expression of the RNAi target gene.

In certain embodiments, said double-stranded RNA complex for down-regulating expression of a mammalian gene comprises (i) a first nucleotide sequence that hybridizes under stringent conditions to a nucleotide sequence of at least one mammalian gene and (ii) a second nucleotide sequence which is complementary to said first nucleotide sequence. In a subgroup of those embodiments, an RNA loop connects the first with the second nucleotide sequence.

A dsRNA complex comprising a nucleotide sequence identical to a portion of the RNAi target gene is preferred for inhibition. RNA sequences with insertions, deletions, and single point mutations relative to the RNAi target sequence have also been found to be effective for inhibition. Thus, sequence identity may be optimized by alignment algorithms known in the art and calculating the percent difference between the nucleotide sequences. Alternatively, the RNA duplex region of the dsRNA complex may be defined functionally as a nucleotide sequence that is capable of hybridizing with a portion of the target gene transcript.

An example for a dsRNA complex are siRNAs. RNAi-inducing dsRNA complexes based on siRNAs are described, for example, in Bummelkamp et al. Science 296:550-553 (2202), Caplen et al. Proc. Natl. Acad. Sci. USA 98:9742-9747 (2001) and Paddison et al. Genes & Devel. 16:948-958 (2002). The dsRNA complex is generally at least about 15 base pairs in length and is preferably about 15 to about 30 base pairs in length. However, a significantly longer dsRNA complex can be used effectively in some organisms. In a more preferred embodiment, the dsRNA complex is between about 19 and 22 base pairs in length. The dsRNA complex is preferably identical to the target nucleotide sequence over this region. When the gene to be down-regulated is in a family of highly conserved genes, the sequence of the duplex region can be chosen with the aid of sequence comparison to target only the desired gene. On the other hand, if there is sufficient identity among a family of homologous genes within an organism, a duplex region can be designed that would down regulate a plurality of genes simultaneously. The RNA duplexes may be flanked by single stranded regions on one or both sides of the duplex. For example, in the case of the hairpin, the single stranded loop region would connect the duplex region at one end.

For purposes of this invention, the term “RNA duplex” or “RNA duplex region” means the part of the dsRNA complex that is homologous and/or complementary to the RNAi target region. In certain embodiments, the RNA duplex might comprise the whole dsRNA complex. The RNA duplex is substantially homologous and/or complementary (typically at least about 80% identical, more preferably at least about 90% identical) in sequence to the RNAi target sequence of the gene targeted for down regulation via RNA interference.

(2) General Methods

The present invention relates to methods for decreasing gene expression by administering to a mammalian subject a recombinant adeno-associated viral vector in vivo with said vector comprising an RNA interference (RNAi) expression cassette whose RNA expression products directly or indirectly lead to a decrease in expression of the corresponding RNAi target gene. Upon successful transduction with the recombinant adeno-associated viral vector, the RNA expression products of the RNAi expression cassette will decrease the cellular concentration of the RNAi target gene mRNA transcripts, thus resulting in decreased concentration of the protein encoded by the RNAi target gene.

The practice of the present invention will employ, unless otherwise indicated, conventional methods of virology, microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)Numerous experimental methods are relevant to this invention or experiments leading thereto, which are within routine skill in the art. These include: methods for isolating nucleic acid molecules, including, for example, phenol chloroform extraction, quick lysis and capture on columns [Kramvis et al., 1996; Sambrook et al., 1989, U.S. Pat. No. 5,582,988 and Yong et al. (1995)]; methods of detecting and quantitating nucleic acid molecules; methods of detecting and quantitating catalytic nucleic acid activity; methods of amplifying a nucleic acid sequence including, for example, PCR, SDA and TMA (also known as (SSR))[Chehab et al., 1987; Fahy et al., 1991; Jonas, V., et al., 1993; Saiki et al., 1985; U.S. Pat. Nos. 4,683,202; 4,683,195; 4,000,159; 4,965,188; 5,176,995; Walder et al., 1993; Walker et al., 1992]; and methods of determining whether a catalytic nucleic acid molecule cleaves an amplified nucleic acid segment including, by way of example, polyacrylamide gel electrophoresis and fluorescence resonance energy transfer (FRET) [Cuenoud and Szostak, 1995; and PCT International Publication No. WO 94/29481].

(2.1) Recombinant AAV Virions

The recombinant AAV virions of the preferred embodiment, comprising an RNAi expression cassette, can be produced using standard methodology, known to the artisan. The methods generally involve the steps of
(1) introducing an AAV vector construct into a host cell (e.g., 293 cells);
(2) introducing an AAV packaging construct into the host cell, where the packaging construct includes AAV coding regions (e.g., rep and cap sequences) capable of being expressed in the host cell to complement AAV packaging functions missing from the AAV vector construct;
(3) introducing one or more helper viruses and/or accessory function vector constructs into the host cell, wherein the helper virus and/or accessory function vector constructs provide accessory functions capable of supporting efficient recombinant AAV (“rAAV”) virion production in the host cell; and
(4) culturing the host cell to produce rAAV virions.

The AAV vector construct, AAV packaging construct and the helper virus or accessory function vector constructs can be introduced into the host cell either simultaneously or serially, using standard transfection techniques.

In one embodiment, pseudotyped rAAV virions are produced, in which a non-AAV5 serotype ITR based RNAi expression cassette is packaged in an AAV5 capsid. The inventors have previously found that this pseudotyping can be achieved by utilizing a Rep protein (or a functional portion thereof) of the same serotype or a cross-reactive serotype as that of the ITRs found in the minigene in the presence of sufficient packaging and accessory functions to permit packaging [83]. Thus, an AAV2 minigene (harboring an RNAi expression cassette) can be pseudotyped in an AAV5 capsid by use of a rep protein from AAV2 or a cross-reactive serotype, e.g., AAV1, AAV3, AAV4 or AAV6. Similarly, an AAV minigene containing AAV1 5′ ITRs and AAV2 3′ ITRs may be pseudotyped in an AAV5 capsid by use of a Rep protein from AAV 1, AAV2, or another cross-reactive serotype. However, because AAV5 is not cross-reactive with the other AAV serotypes, an AAV5 minigene can be pseudotyped in a heterologous AAV capsid only by use of an AAV5 Rep protein.

In certain embodiments, the invention provides an rAAV virion, in which both the AAV ITRs and capsid protein are independently selected from among AAV serotypes, including, without limitation, AAV1, AAV2, AAV3, AAV4, AAV5, AAV6, AAV7, AAV8. For example, the invention may utilize a rAAV1 vector, a rAAV2 vector, a rAAV2/1 vector, and rAAV1/2 vector and/or a rAAV2/5 vector, as desired following the nomenclature rAAVx/y with x: serotype source of ITRs, y: serotype source of capsid; rAAVz with z as serotype source of ITRs and capsid.

In another embodiment of this method, the delivery of vector with an AAV capsid protein may precede or follow delivery of a heterologous molecule (e.g., gene) via a vector with a different serotype AAV capsid protein. Thus, delivery via multiple rAAV vectors may be used for repeat delivery of a desired molecule to a selected host cell. Desirably, subsequently administered rAAV carry the same minigene as the first rAAV vector, but the subsequently administered vectors contain capsid proteins of serotypes which differ from the first vector. For example, if a first rAAV has an AAV5 capsid protein, subsequently administered rAAV may have capsid proteins selected from among the other serotypes, including AAV2, AAV1, AAV3A, AAV3B, AAV4 and AAV6.Alternatively, if a first rAAV has an AAV2 capsid protein, subsequently administered rAAV may have an AAV5 capsid. Still other suitable combinations will be readily apparent to one of skill in the art.

The host cell for rAAV virion production itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, W138, HeLa, 293 cells (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc. The requirements for the cell used is that it not carry any adenovirus gene other than E1, E2a and/or E4 ORF6; it not contain any other virus gene which could result in homologous recombination of a contaminating virus during the production of rAAV; and it is capable of infection or transfection of DNA and expression of the transfected DNA.

One host cell useful in the present invention is a host cell stably transformed with the sequences encoding rep and cap, and which is transfected with the adenovirus E1, E2a, and E40RF6 DNA and a construct carrying the minigene as described above. Stable rep and/or cap expressing cell lines, such as B-50 (PCT/US98/19463), or those described in U.S. Pat. No. 5,658,785, may also be similarly employed. Another desirable host cell contains the minimum adenoviral DNA which is sufficient to express E4 ORF6.

The preparation of a host cell according to this invention involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods which provide the desired nucleotide sequence.

Introduction of the molecules (as plasmids or viruses) into the host cell may also be accomplished using techniques known to the skilled artisan and as discussed throughout the specification. In the preferred embodiment, standard transfection techniques are used, e.g., CaPO.sub.4 transfection or electroporation, and/or infection by hybrid adenovirus/AAV vectors into cell lines such as the human embryonic kidney cell line HEK 293 (a human kidney cell line containing functional adenovirus E1 genes which provides trans-acting E1 proteins). Thus produced, the rAAV may be used to prepare the compositions and kits described herein, and used in the method of the invention.

(2.1.1) AAV Vector Constructs

AAV vector constructs are constructed using known techniques to at least provide, as operatively linked components in the direction of transcription, (a) control elements including a transcriptional initiation region, (b) the DNA of interest (here: at least an RNAi expression cassette), and (c) a transcriptional termination region. The control elements are selected to be functional in the targeted cell. The resulting construct, which contains the operatively linked components, is bounded (5′ and 3′) with functional AAV ITR sequences. The nucleotide sequences of AAV ITR regions are known. See, e.g., [84]; Berns, K. I. “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides.

Additionally, AAV ITRs may be derived from any of several AAV serotypes, including AAV-1, AAV-2, AAV-3, AAV-4, AAV-5, AAV-6, AAV-7, AAV-8, etc. The 5′ and 3′ ITRs which flank a selected transgene expression cassette in an AAV vector plasmid need not necessarily be identical or derived from the same AAV serotype, as long as they function as intended, i.e., to allow for excision and replication of the bounded nucleotide sequence of interest when AAV rep gene products are present in the cell. Thus, rAAV vector design and production allows for exchanging of the capsid proteins between different AAV serotypes: Homologous vectors comprising an expression cassette flanked by e.g., AAV2-ITRs and packaged in an AAV2 capsid, can be produced as well as heterologous, hybrid vectors where the transgene expression cassette is flanked by e.g., AAV2 ITRs, but the capsid originates from another AAV serotype: The following combinations are feasible: rAAV2/1-8, where the first number defines the genome and the second the capsid of the AAV of origin. In its preferred embodiment, the gene transfer vector is produced using a rAAV2/5 design.

Suitable minigenes for use in AAV vectors will generally be less than about 5 kilobases (kb) in size, which is the case for RNAi expression cassettes. Given the size of most RNAi expression cassettes, other minigenes might be included in the same AAV vector comprising another gene of interest.

The AAV sequences used in generating the minigenes, vectors, and capsids, and other constructs used in the present invention may be obtained from a variety of sources. For example, the sequences may be provided by AAV type 5, AAV type 2, AAV type 1, AAV type 3, AAV type 4, AAV type 6, or other AAV serotypes or other denso-viruses. A variety of these viral serotypes and strains are available from the American Type Culture Collection, Manassas, Va., or are available from a variety of academic or commercial sources. Alternatively, it may be desirable to synthesize sequences used in preparing the vectors and viruses of the invention using known techniques, which may utilize AAV sequences which are published and/or available from a variety of databases. The source of the sequences utilized in preparation of the constructs of the invention is not a limitation of the present invention.

(2.1.2) rAAV Virion Production

In order to produce rAAV virions, an AAV vector construct that has been constructed as described above is introduced into a suitable host cell using known techniques, such as by transfection. A number of transfection techniques are generally known in the art. See, e.g., [69, 70], Sambrook et al. (1989) Molecular Cloning, a laboratory manual, Cold Spring Harbor Laboratories, New York, Davis et al. (1986) Basic Methods in Molecular Biology, Elsevier, and [71]. Particularly suitable transfection methods include calcium phosphate co-precipitation [69], direct micro-injection into cultured cells [85], electroporation [86], liposome mediated gene transfer [87], lipid-mediated transduction [88], and nucleic acid delivery using high-velocity microprojectiles.

The AAV vector construct harboring the AAV minigene is preferably carried on a plasmid which is delivered to a host cell by transfection. The plasmids useful in this invention may be engineered such that they are suitable for replication and, optionally, integration in prokaryotic cells, mammalian cells, or both. These plasmids (or other vectors carrying the 5′ AAV ITR-heterologous molecule-3′ AAV ITR) may contain sequences permitting replication of the AAV minigene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the AAV minigene is transfected into the cell, where it may exist transiently or as an episome. Alternatively, the AAV minigene (carrying the 5′ AAV ITR-heterologous molecule-3′ AAV ITR) may be stably integrated into a chromosome of the host cell. Suitable transfection techniques are known and may readily be utilized to deliver the AAV minigene to the host cell.

Generally, when delivering the AAV vector construct comprising the AAV minigene by transfection, the vector is delivered in an amount from about 5 .mu.g to about 100 .mu.g DNA, and preferably about 10 to about 50 .mu.g DNA to about 1.times.10.sup.4 cells to about 1.times.10.sup.13 cells, and preferably about 10.sup.5 cells. However, the relative amounts of vector DNA to host cells may be adjusted, taking into consideration such factors as the selected vector, the delivery method and the host cells selected.

For the purposes of the invention, suitable host cells for producing rAAV virions include microorganisms, yeast cells, insect cells, and mammalian cells, that can be, or have been, used for transfection. The term includes the progeny of the original cell which has been transfected. Thus, a “host cell” as used herein generally refers to a cell which has been transfected with an exogenous DNA sequence. Cells from the stable human cell line, 293 (readily available through, e.g., the ATCC under Accession No. ATCC CRL1573) are preferred in the practice of the present invention. Particularly, the human cell line 293 is a human embryonic kidney cell line that has been transformed with adenovirus type-5 DNA fragments [89], and expresses the adenoviral E1a and E1b genes [90]. The 293 cell line is readily transfected, and provides a particularly convenient platform in which to produce rAAV virions.

The components required to be cultured in the host cell to package the AAV minigene in the AAV capsid may be provided to the host cell in trans. Alternatively, any one or more of the required components (e.g., minigene, rep sequences, cap sequences, and/or accessory functions) may be provided by a stable host cell which has been engineered to contain one or more of the required components using methods known to those of skill in the art.

The minigene, rep sequences, cap sequences, and accessory (helper) functions required for producing the rAAV of the invention may be delivered to the packaging host cell in the form of any genetic element, e.g., naked DNA, a plasmid, phage, transposon, cosmid, virus, etc. which transfer the sequences carried thereon. The selected genetic element may be delivered by any suitable method, including transfection, electroporation, liposome delivery, membrane fusion techniques, high velocity DNA-coated pellets, viral infection and protoplast fusion.

(2.1.3) AAV Packaging Functions

Host cells containing the above described AAV vector constructs must be rendered capable of providing AAV packaging functions in order to replicate and encapsidate the nucleotide sequences flanked by the AAV ITRs to produce rAAV virions. AAV packaging functions are generally AAV-derived coding sequences which can be expressed to provide AAV gene products that, in turn, function in trans for productive AAV replication and genome encapsidation. AAV packaging functions are used herein to complement necessary AAV functions that are missing from the AAV vectors. Thus, AAV packaging functions include one, or both of the major AAV ORFs, namely the rep and cap coding regions, or functional homologues thereof.

By “AAV rep coding region” is meant the art-recognized region of the AAV genome which encodes the replication proteins Rep78, Rep68, Rep52 and Rep40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., [84, 91]. Suitable homologues of the AAV rep coding region include the human herpesvirus 6 (HHV-6) rep gene which is also known to mediate AAV-2 DNA replication [92].

By “AAV cap coding region” is meant the art-recognized region of the AAV genome which encodes the capsid proteins VP1, VP2, and VP3, or functional homologues thereof. These cap expression products supply the packaging functions which are collectively required for packaging the viral genome. For a description of the AAV cap coding region, see, e.g., [84, 91].

AAV packaging functions are introduced into the host cell by transfecting the host cell with an AAV packaging construct either prior to, or concurrently with, the transfection of the AAV vector construct. AAV packaging constructs are thus used to provide at least transient expression of AAV rep and/or cap genes to complement missing AAV functions that are necessary for productive AAV infection. AAV packaging constructs lack AAV ITRs and can neither replicate nor package themselves. These constructs can be in the form of a plasmid, phage, transposon, cosmid, virus, or virion. A number of AAV packaging constructs have been described, such as the commonly used plasmids pAAV/Ad and pIM29+45 which encode both Rep and Cap expression products. See, e.g., [93, 94]. A number of other vectors have been described which encode Rep and/or Cap expression products. See, e.g., U.S. Pat. No. 5,139,941.

Additionally, when pseudotyping an AAV vector in an AAV5 capsid, the sequences encoding each of the essential Rep proteins may be supplied by the same AAV serotype as the ITRs, or the sequences encoding the Rep proteins may be supplied by different, but cross-reactive, AAV serotypes (e.g., AAV1, AAV2, AAV3, AAV4 and AAV6). For example, the Rep78/68 sequences may be from AAV2, whereas the Rep52/40 sequences may from AAV1.

In one embodiment, the host cell stably contains the capsid ORF under the control of a suitable promoter, such as those described above. Most desirably, in this embodiment, the capsid ORF is expressed under the control of an inducible promoter. In another embodiment, the capsid ORF is supplied to the host cell in trans. When delivered to the host cell in trans, the capsid ORF may be delivered via a plasmid which contains the sequences necessary to direct expression of the selected capsid ORF in the host cell. Most desirably, when delivered to the host cell in trans, the plasmid carrying the capsid ORF also carries other sequences required for packaging the rAAV, e.g., the rep sequences.

In another embodiment, the host cell stably contains the rep sequences under the control of a suitable promoter, such as those described above. Most desirably, in this embodiment, the essential Rep proteins are expressed under the control of an inducible promoter. In another embodiment, the rep ORF is supplied to the host cell in trans. When delivered to the host cell in trans, the rep ORF may be delivered via a plasmid which contains the sequences necessary to direct expression of the selected rep ORF in the host cell. Most desirably, when delivered to the host cell in trans, the plasmid carrying the rep ORF also carries other sequences required for packaging the rAAV, e.g., the cap sequences.

Thus, in one embodiment, the rep and cap sequences may be transfected into the host cell on a single nucleic acid molecule and exist in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

Optionally, the rep and/or cap sequences may be supplied on a vector that contains other DNA sequences that are to be introduced into the host cells. For instance, the vector may contain the rAAV vector construct comprising the AAV minigene. The vector may comprise one or more of the genes encoding the helper functions, e.g., the adenoviral proteins E1, E2a, and E40RF6, and the gene for VAI RNA.

In another embodiment, the promoter for rep is an inducible promoter, as discussed above in connection with regulatory sequences and promoters. One preferred promoter for rep expression is the T7 promoter. The vector comprising the rep gene regulated by the T7 promoter and the cap gene, is transfected or transduced into a cell which either constitutively or inducibly expresses the T7 polymerase. See WO 98/10088, published Mar. 12, 1998.

Preferably, the promoter used in the AAV packaging construct may be any of the constitutive, inducible or native promoters known to one of skill in the art or as discussed above. In one embodiment, an AAV p5 promoter sequence is employed. The selection of the AAV to provide any of these sequences does not limit the invention.

The spacer is an optional element in the design of the AAV packaging construct. The spacer is a DNA sequence interposed between the promoter and the rep gene ATG start site. The spacer may have any desired design; that is, it may be a random sequence of nucleotides, or alternatively, it may encode a gene product, such as a marker gene. The spacer may contain genes that typically incorporate start/stop and polyA sites. The spacer may be a non-coding DNA sequence from a prokaryote or eukaryote, a repetitive non-coding sequence, a coding sequence without transcriptional controls or a coding sequence with transcriptional controls. Two exemplary sources of spacer sequences are the X phage ladder sequences or yeast ladder sequences, which are available commercially, e.g., from Gibco or Invitrogen, among others. The spacer may be of any size sufficient to reduce expression of the rep78 and rep68 gene products, leaving the rep52, rep40 and cap gene products expressed at normal levels. The length of the spacer may therefore range from about 10 bp to about 10.0 kbp, preferably in the range of about 100 bp to about 8.0 kbp. To reduce the possibility of recombination, the spacer is preferably less than 2 kbp in length; however, the invention is not so limited.

Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell. The methods employed for constructing embodiments of this invention are conventional genetic engineering or recombinant engineering techniques such as those described in the references above. While this specification provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

(2.1.4) AAV Accessory Functions

The host cell (or packaging cell) must also be rendered capable of providing non-AAV derived functions, or “accessory functions”, in order to produce rAAV virions. Accessory functions are non-AAV derived viral and/or cellular functions upon which AAV is dependent for its replication. Thus, accessory functions include at least those non AAV proteins and RNAs that are required in AAV replication, including those involved in activation of AAV gene transcription, stage specific AAV mRNA splicing, AAV DNA replication, synthesis of rep and cap expression products and AAV capsid assembly. Viral-based accessory functions can be derived from any of the known helper viruses.

Particularly, accessory functions can be introduced into and then expressed in host cells using methods known to those of skill in the art. Commonly, accessory functions are provided by infection of the host cells with an unrelated helper virus. A number of suitable helper viruses are known, including adenoviruses, Herpes viruses such as Herpes Simplex Virus types 1 and 2, and vaccinia viruses. Non-viral accessory functions will also find use herein, such as those provided by cell synchronization using any of various known agents [95-97]. Alternatively and preferentially, accessory functions can be provided using an accessory function vector construct. Accessory function vector constructs include nucleotide sequences that provide one or more accessory functions. An accessory function vector is capable of being introduced into a suitable host cell in order to support efficient AAV virion production in the host cell. Accessory function vectors can be in the form of a plasmid, phage, virus, transposon or cosmid. Accessory vector constructs can also be in the form of one or more linearized DNA or RNA fragments which, when associated with the appropriate control elements and enzymes, can be transcribed or expressed in a host cell to provide accessory functions.

Nucleic acid sequences providing the accessory functions can be obtained from natural sources, such as from the genome of adenovirus (especially Adenovirus serotype 5), or constructed using recombinant or synthetic methods known in the art. In this regard, adenovirus-derived accessory functions have been widely studied, and a number of adenovirus genes involved in accessory functions have been identified and partially characterized. See, e.g., Carter, B. J. (1990) “Adeno-Associated Virus Helper Functions,” in CRC Handbook of Parvoviruses, vol. I (P. Tijssen, ed.), and [91]. Specifically, early adenoviral gene regions E1a, E2a, E4, VAI RNA and, possibly, E1b are thought to participate in the accessory process [98]. Herpes Virus-derived accessory functions have been described as well [99]. Vaccinia virus-derived accessory functions have also been described [95].

Most desirably, the necessary accessory functions are provided from an adenovirus source. In one embodiment, the host cell is provided with and/or contains an E1a gene product, an E1b gene product, an E2a gene product, and/or an E4 ORF6 gene product. The host cell may contain other adenoviral genes such as VAI RNA, but these genes are not required. In a preferred embodiment, no other adenovirus genes or gene functions are present in the host cell. The DNA sequences encoding the adenovirus E4 ORF6 genes and the E1 genes and/or E2a genes useful in this invention may be selected from among any known adenovirus type, including the presently identified 46 human types [see, e.g., American Type Culture Collection]. Similarly, adenoviruses known to infect other animals may supply the gene sequences. The selection of the adenovirus type for each E1, E2a, and E4 ORF6 gene sequence does not limit this invention. The sequences for a number of adenovirus serotypes, including that of serotype Ad5, are available from Genbank. A variety of adenovirus strains are available from the American Type Culture Collection (ATCC), Manassas, Va., or are available by request from a variety of commercial and institutional sources. Any one or more of human adenoviruses Types 1 to 46 may supply any of the adenoviral sequences, including E1, E2a, and/or E4 ORF6.

The adenovirus E1a, E1b, E2a, and/or E40RF6 gene products, as well as any other desired accessory functions, can be provided using any means that allows their expression in a cell. Each of the sequences encoding these products may be on a separate vector, or one or more genes may be on the same vector. The vector may be any vector known in the art or disclosed above, including plasmids, cosmids and viruses. Introduction into the host cellof the vector may be achieved by any means known in the art or as disclosed above, including transfection, infection, electroporation, liposome delivery, membrane fusion techniques, high velocity DNA-coated pellets, viral infection and protoplast fusion, among others. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiationstate or in replicating or quiescent cells) or by exogenously-added factors, for example.

As a consequence of the infection of the host cell with a helper virus, or transfection of the host cell with an accessory function vector construct, accessory functions are expressed which transactivate the AAV packaging construct to produce AAV Rep and/or Cap proteins. The Rep expression products direct excision of the recombinant DNA (including the DNA of interest) from the AAV vector construct. The Rep proteins also serve to replicate the AAV genome. The expressed Cap proteins assemble into capsids, and the recombinant AAV genome is packaged into the capsids. Thus, productive AAV replication ensues, and the DNA is packaged into rAAV virions.

Following recombinant AAV replication, rAAV virions can be purified from the host cell using a variety of conventional purification methods, such as CsCl gradients or column purification. Further, if helper virus infection is employed to express the accessory functions, residual helper virus can be inactivated, using known methods. For example, adenovirus can be inactivated by heating to temperatures of approximately 60.degree. C. for, e.g., 20 minutes or more. This treatment selectively inactivates the helper virus which is heat labile, while preserving the rAAV which is heat stable. The resulting rAAV virions are then ready for use for DNA delivery to a variety of target cells.

(2.2) In vivo Delivery of rAAV Virions and Pharmaceutical Compositions

The present invention relates to a method for the transfer of nucleic acid compositions to the cells of an individual in general and to the transfer of RNAi expression cassettes in particular. The method comprises the step of contacting cells of said individual with rAAV-based gene transfer vectors which include at least one RNAi expression cassette, thereby delivering said RNAi expression cassette to the nucleus within said cells. The rAAV vectors are administered to the cells of said individual on an in vivo basis, i.e., the contact with the cells of the individual takes place within the body of the individual in accordance with the procedures which are most typically employed.

The rAAV virion is preferably suspended in a pharmaceutically acceptable delivery vehicle (i.e., physiologically compatible carrier), for administration to a human or non-human mammalian patient. Suitable carriers may be readily selected by one of skill in the art and may depend on the nature of the nucleic acid transfer vector chosen. Pharmaceutical compositions will comprise sufficient genetic material to produce a therapeutically effective amount of dsRNA complexes. The pharmaceutical compositions will also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. Other exemplary carriers include lactose, sucrose, calcium phosphate, gelatin, dextran, agar, pectin, peanut oil, sesame oil, and water. The selection of the carrier is not a limitation of the present invention. Optionally, the compositions of the invention may contain, in addition to the rAAV virions and carrier(s), other conventional pharmaceutical ingredients, such as preservatives, or chemical stabilizers. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin and albumin. A thorough discussion of pharmaceutically acceptable excipients is available in REMINGTON'S PHARMACEUTICAL SCIENCES (Mack Pub. Co., N.J. 1991).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

In this invention, administering the instant pharmaceutical composition can be effected or performed using any of the various methods and delivery systems known to those skilled in the art. The administering can be performed, for example, intravenously, orally, via implant, transmucosally, transdermally, intramuscularly, and subcutaneously. In addition, the instant pharmaceutical compositions ideally contain one or more routinely used pharmaceutically acceptable carriers. Such carriers are well known to those skilled in the art. The following delivery systems, which employ a number of routinely used carriers, are only representative of the many embodiments envisioned for administering the instant composition.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Determining a therapeutically or prophylactically effective amount of the instant pharmaceutical composition can be done based on animal data using routine computational methods. Appropriate doses will depend, among other factors, on the specifics of the transfer vector chosen, on the route of administration, on the mammal being treated (e.g., human or non-human primate or other mammal), age, weight, and general condition of the subject to be treated, the severity of the cancer being treated, the location of the cancer being treated and the mode of administration. Thus, the appropriate dosage may vary from patient to patient. An appropriate effective amount can be readily determined by one of skill in the art. In one specific embodiment, the nucleic acid transfer vector is an AAV2/5 hybrid vector. A therapeutically effective human dosage for in vivo delivery of said vector according to the present invention is believed to be in the range of from about 20 to about 50 ml of saline solution containing concentrations of from about 10¹⁰ to 10¹⁴ functional vector/ml solution. The dosage will be adjusted to balance the therapeutic benefit against any side effects. In yet another embodiment, pharmaceutically effective dose of the rAAV is generally in the range of concentrations of from about 1.times.10.sup.5 to 1.times.10.sup.50 genomes rAAV, about 10.sup.8 to 10.sup.20 genomes rAAV, about 10.sup.10 to about 10.sup.16 genomes, or about 10.sup.11 to 10.sup.16 genomes rAAV. A preferred human dosage may be about 1.times.10.sup.13 AAV genomes rAAV. Such concentrations may be delivered in about 0.001 ml to 100 ml, 0.05 to 50 ml, or 10 to 25 ml of a carrier solution. Other effective dosages can be readily established by one of ordinary skill in the art through routine trials establishing dose response curves.

Dosage treatment may be a single dose schedule or a multiple dose schedule. Moreover, the subject may be administered as many doses as appropriate. One of skill in the art can readily determine an appropriate number of doses. However, the dosage may need to be adjusted to take into consideration an alternative route of administration, or balance the therapeutic benefit against any side effects. Such dosages may vary depending upon the therapeutic application for which the recombinant vector is employed.

The vector particles are administered in sufficient amounts to enter the desired cells and to guarantee sufficient levels of functionality of the transferred nucleic acid composition to provide a therapeutic benefit without undue adverse, or with medically acceptable, physiological effects which can be determined by those skilled in the medical arts.

In some embodiments, conventional pharmaceutically acceptable routes of administration of rAAV may be combined. These routes include, but are not limited to, direct delivery to the liver, intravenous, intramuscular, subcutaneous, intradermal, oral and other parental routes of administration.

Optionally, in specific embodiments, rAAV-mediated delivery according to the invention may be combined with delivery by other viral and non-viral vectors. Such other viral vectors including, without limitation, adenoviral vectors, retroviral vectors, lentiviral vectors. herpes simplex virus (HSV) vectors, and baculovirus vectors may be readily selected and generated according to methods known in the art. Similarly, non-viral vectors, including, without limitation, liposomes, lipid-based vectors, polyplex vectors, molecular conjugates, polyamines and polycation vectors, may be readily selected and generated according to methods known in the art. When administered by these alternative routes, the dosage is desirable in the range described above.

In one embodiment, the route of administration is inhalation with lung cells as RNAi target cells. In that instance, when prepared for use as an inhalant, the pharmaceutical compositions are prepared as fluid unit doses using the rAAV and a suitable pharmaceutical vehicle for delivery by an atomizing spray pump, or by dry powder for insufflation. For use as aerosols, the rAAV can be packaged in a pressurized aerosol container together with a gaseous or liquefied propellant, for example, dichlorodifluormethane, carbon dioxide, nitrogen, propane, and the like, with the usual components such as cosolvents and wetting agents, as may be necessary or desirable. A pharmaceutical kit of said embodiment, desirably contains a container for oral or intranasal inhalation, which delivers a metered dose in one, two, or more actuations. Suitably, the kit also contains instructions for use of the spray pump or other delivery device, instructions on dosing, and an insert regarding the active agent (i.e., the transgene and/or rAAV). A single actuation of a pump spray or inhaler generally delivers contains in the range of about 10.sup.5 to about 10.sup.15 genome copies (GC), about 10.sup.8 to about 10.sup.12, and/or about 10.sup.10 GC, in a liquid containing 10 .mu.g to 250 .mu.g carrier, 25 .mu.g to 100 .mu.g, or 40 .mu.g to 50 .mu.g, carrier. Suitably, a dose is delivered in one or two actuations. However, other suitable delivery methods may be readily determined. The doses may be repeated daily, weekly, or monthly, for a predetermined length of time or as prescribed.

(2.3) RNAi expression cassettes

In designing an RNAi expression cassette one has to make several choices in respect to the

(1) length of the dsRNA complex to be formed inside the cell during or upon expression of the RNA transcript(s) of the RNAi expression cassette(s)

(2) RNAi Target Sequence

(3) Design of the RNAi expression cassette: (3.1) Nature of the dsRNA complex (e.g., a dsRNA complex formed intermolecularly by two RNA molecules, or formed intramolecularly by one RNA molecule); (3.2) Selection and arrangement of regulatory sequences (e.g., choice of promoter and other regulatory sequences); (3.3) Choice of the number of rAAV vectors (e.g., use of two rAAV vectors each comprising one RNAi expression cassette, or use of one rAAV vector comprising an RNAi expression cassette).

To (1): Length of the dsRNA to be Formed Inside the Cell

Animals (including humans) possess a natural defense mechanism against pathogens with dsRNA genomes: The presence of dsRNA in the cytosol induces the activation of Interferon-related pathways, which suppress RNA interference. Thus, in order to avoid the activation of those pathways, the dsRNA complex should not exceed 30 base pairs in length—the prime rule in designing siRNA constructs [31].

In certain preferred embodiments, the length of the dsRNA complex is at least 20, 21 or 22 base pairs in length, e.g., corresponding in size to RNA products produced by Dicer-dependent cleavage. In certain embodiments, the dsRNA complex is at least 25, 50, 100, 200, 300 or 400 base pairs. In certain embodiments, the dsRNA complex is 400-800 base pairs in length.

The length of the linear RNA molecule must be sufficient to give rise to a dsRNA complex that is at least about 20 base pairs in length. Although there is no upper limit to the length of the linear RNA molecule, in one embodiment, the RNA molecule is about 20 and 3000 nucleotides in length (allowing for about 10 and 1500 base pairs of dsRNA). In another embodiment, the RNA molecule is between about 35 and 55 nucleotides in length. In yet another embodiment, the RNA molecule is about 100 and 1000 nucleotides in length.

In one embodiment, the RNAi expression cassette encodes an RNA molecule from about 14 to about 50 nucleotides in length. In another embodiment, the RNA molecule encoded by the RNAi expression cassette is about 14, 15, 16, 17, 18, 19, 20, 21, 22, 23, 24, 25, 26, 27, or 28 nucleotides in length. In yet another embodiment, the RNA molecule encoded by the RNAi expression cassette is about 23 nucleotides in length. In one embodiment, the RNA molecule encoded by the RNAi expression cassette is from about 28 to about 56 nucleotides in length. In another embodiment, the RNA molecule encoded by the RNAi expression cassette is about 40, 41, 42, 43, 44, 45, 46, 47, 48, 49, 50, 51, or 52 nucleotides in length. In yet another embodiment, an RNA molecule of the invention is about 46 nucleotides in length.

To (2): RNAi Target Sequence

The objective of expressing a dsRNA complex is to down-regulate the expression a specific gene in a mammalian cell or organism in vivo. Thus, the RNAi target sequence has to be selected with care: It should not be homologous to any other gene (in order to avoid side effects by unintended down-regulation of other genes). According to Vickers et al. [100], it might also be preferable to target an mRNA stretch with a lower degree of secondary structure. Interestingly, in the same report, Vickers et al. present data suggesting that siRNA activity is primarily cytoplasmic and therefore does not interact with pre-mRNA. Thus, designing siRNAs with complementarity to intronic sequences might show lower efficacy.

To summarize: The RNAi target sequence should be unique and preferentially in an exonic area with low degree of secondary structure.

A dsRNA complex containing a nucleotide sequences identical to a portion, of either coding or non-coding sequence, of the target gene are preferred for inhibition. RNA sequences with insertions, deletions, and single point mutations relative to the RNAi target sequence have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the RNA duplex and the portion of the RNAi target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50.degree. C. or 70.degree. C. hybridization for 12-16 hours; followed by washing).

The sequence of the RNA coding region, and thus the sequence of the dsRNA complex and RNA duplex, preferably is chosen to be complementary to the sequence of a gene whose expression is to be down-regulated in a cell or organism. The degree of down-regulation achieved with a given RNA duplex sequence for a given RNAi target gene will vary by sequence. One of skill in the art will be able to readily identify an effective sequence. For example, in order to maximize the amount of suppression in a mammalian subject, a number of sequences can be tested for their efficacy in cell culture. As an understanding of the sequence requirements for RNA interference is determined, the RNA duplex can be selected by one of skill in the art.

To (3): Design of the RNAi Expression Cassette

After determining the RNAi target sequence, the artisan can design the RNAi expression cassette without undue effort. Some basic design principles have already been discussed in prior art:(1)The dsRNA complex can be formed intramolecularly by a single RNA molecule as a stem-loop structure (similar to the synthesis of stRNAs and miRNAs [41, 42, 73, 101]) (2)The dsRNA complex can be formed intermolecularly by two RNA molecules, e.g., by using dual promoters to transcribe a sense- and antisense strand separately [43, 102].

The RNAi expression cassette generally comprises at least(1) a promoter, operative in the RNAi target cell, (2) an RNA coding region, (3)a DNA region comprising transcription termination and/or polyadenylation signals.

The RNAi expression cassette preferably comprises an RNA coding region operably linked to the RNA Polymerase III promoter. The RNA coding region preferably comprises a DNA sequence that can serve as a template for the expression of a desired RNA molecule. The RNA coding region can be immediately followed by a pol III terminator sequence which directs the accurate and efficient termination of RNA synthesis by pol III. The pol III terminator sequences generally comprise 4 or more consecutive thymidine residues. In a preferred embodiment, a cluster of 5 consecutive thymidines is used as the terminator by which pol III transcription is stopped at the second or third thymidine of the DNA template. As a result, only one to four uracil residues are added to the 3′ end of the RNA that is synthesized from the RNA coding region.

Both the U6 [42] and H1 [73] promoters have been used successfully. One key advantage of using a Pol III system is that transcription terminates at a defined stretch of thymidine residues, leaving one to four uridines at the 3′-terminus of the nascent RNA, thereby making it similar to many siRNAs. A variety of RNA Pol III promoters can be used with the invention, including for example, the promoter fragments derived from H1 RNA genes or U6 sn RNA genes of human or mouse origin or from any other species. In addition, pol III promoters can be modified/engineered to incorporate other desirable properties such as to be inducible by small chemical molecules either ubiquitously or in a tissue-specific manner, for example, one activated with tetracycline or IPTG (lacI system).

In one embodiment, the dsRNA complex is formed by a single RNA molecule as a stem-loop structure, whereas expression of the RNA molecule is under the control of an RNA Pol III promoter.

In its preferred embodiment, the RNA coding region of the RNAi expression cassette encodes a self-complementary RNA molecule having a sense region, an antisense region and a loop region. This RNA molecule might be a siRNA. Such an RNA molecule when expressed desirably forms a dsRNA complex with a “hairpin” structure through intramolecular hybridisation. The loop region is generally between about 2 and about 15 nucleotides in length. In a preferred embodiment, the loop region is from about 6 and about 9 nucleotides in length. In one such embodiment of the invention, the sense region and the antisense region are between about 10 and about 30 nucleotides in length. The stem-loop structures are either modeled after naturally occurring stRNAs or miRNAs or simply linked at one end by a few nucleotides. Many of the early reports suggest that even minor base changes in the stem or loop structure can affect silencing [73, 103]. The RNA duplex region of the hairpin molecule comprises a nucleotide sequence that is homologous to the RNAi target sequence. The sequence in the hairpin molecule is preferably at least about 90% identical to the RNAi target sequence, more preferably at least about 95% identical, even more preferably at least about 99% identical. The sense region is substantially homologous with or complementary to at least part of the nucleotide sequence of the RNAi target gene; the antisense region is substantially complementary to the sense region, thus allowing the RNA molecule to form a dsRNA complex by base pairing between the regions with sense and antisense nucleotide sequence resulting in a hairpin dsRNA structure.

In certain embodiments, the RNAi expression cassette includes a single RNA coding sequence which is operably linked to (two) transcriptional regulatory sequences which cause transcription in both directions to form complementary transcripts of the coding sequence capable of forming the dsRNA complex. In other embodiments, the RNAi expression cassette includes two RNA coding sequences which, respectively, give rise to the two complementary sequences which form the dsRNA complex when annealed.

In some embodiments of the invention, the RNAi expression cassette comprises a first RNA pol III promoter operably linked to a first RNA coding region, and a second RNA pol III promoter operably linked to the same first RNA coding region in the opposite direction, such that expression of the RNA coding region from the first RNA pol III promoter results in a synthesis of a first RNA molecule as the sense strand and expression of the RNA coding region from the second RNA pol III promoter results in synthesis of a second RNA molecule as an antisense strand that is substantially complementary to the first RNA molecule. In one such embodiment, both RNA Polymerase III promoters are separated from the RNA coding region by termination sequences, preferably termination sequences having five consecutive T residues. The methods of invention also include multiple RNA coding regions that encode hairpin-like self-complementary RNA molecules or other non-hairpin molecules.

In some embodiments of the invention, the RNAi expression cassette comprises a first RNA pol III promoter operably linked to a first RNA coding region, and a second RNA pol III promoter operably linked to a second RNA coding region, such that expression of the first RNA coding region from the first RNA pol III promoter results in a synthesis of a first RNA molecule and expression of the second RNA coding region from the second RNA pol III promoter results in synthesis of a second RNA molecule with the second RNA molecule designed to be substantially complementary to the first RNA molecule. Thus, the two RNA molecules will be able to hybridize and form an RNAi inducing dsRNA complex in vivo.

In another embodiment, the RNA coding region of the RNAi expression cassette encodes a linear RNA molecule for forming a double-stranded RNA complex, which RNA molecule comprises(i) a first sequence which, under hybridizing conditions, hybridizes to at least a portion of an mRNA molecule encoded by a gene or a nucleic acid; (ii) a second sequence which, under hybridizing conditions, hybridizes to the first sequence; and (iii) a third sequence situated between the first and second sequences so as to permit the first and second sequences to hybridize with each other, whereby, under hybridizing conditions, they will form a double-stranded RNA complex upon hybridization between the first and second sequences.

In some embodiments, the RNA coding region might further comprise sequences to enhance the efficiency of specific gene regulation. Such sequences capable of enhancing specific regulation by dsRNA would include, but not be restricted to, short viral or cellular dsRNAs (such as adenovirus VAI, HIV-1 TAR, EBER-1, and Alu RNAs).

In yet another embodiment a fourth sequence on a linear RNA molecule is provided that includes (i) a ribozyme and (ii) a ribozyme target sequence specifically recognized by the ribozyme and absent in the sense, antisense and loop region, whereby the complex-forming sense and anti-sense regions forms a double-stranded RNA complex upon hybridization and the ribozyme target sequence is cleaved by the ribozyme. The fourth sequence may also comprise a plurality of ribozymes and ribozyme target sequences cleaved thereby. Thus, the order of RNA regions within the linear RNA molecule comprises: sense region—loop region—antisense region—ribozyme(s) region with the ribozyme region comprised of the ribozyme target sequence and the ribozyme sequence. This design has the advantage of using Pol II promoters without having a polyadenylated RNA region as a result as the ribozyme will be able to remove itself and the polyadenylated region from the dsRNA complex.

The ribozyme can be any type of ribozyme. In the preferred embodiment, the ribozyme is a hammerhead ribozyme. Within the parameters of this invention, the binding domain lengths (also referred to herein as “arm lengths”) of a ribozyme can be of any permutation, and can be the same or different. Various permutations such as 7+7, 8+8 and 9+9 bases/nucleotides are envisioned. It is well established that the greater the binding domain length, the more tightly it will bind to its complementary mRNA sequence. According, in the preferred embodiment, each binding domain is nine nucleotides in length. A preferred ribozyme is a cis-acting hammerhead ribozyme.

In yet another embodiment of the invention, two rAAV vectors are used each comprising its own RNAi expression cassette. The RNAi expression cassette of the first vector comprises a first RNA pol III promoter, a first RNA coding region encoding a first RNA molecule operably linked to the first RNA pol III promoter; the RNAi expression cassette of the second vector comprises a second RNA pol III promoter and a second RNA coding region operably linked to the second RNA pol III promoter. Preferably, the RNA coding region of the second vector encodes an RNA molecule that is substantially complementary to the RNA molecule encoded by the first RNA coding region of the first vector, such that the two RNA molecules can form a double-stranded RNA complex when expressed.

In yet another embodiment of this invention, the invention relates to a linear RNA molecule encoded by an RNAi expression cassette capable of forming a dsRNA complex wherein the RNA molecule comprises: (a) a first portion that comprises a region of RNA that is complementary to at least a portion of a mRNA molecule encoded by a gene; (b) a second portion capable of hybridizing to at least part of the first portion; and (c) a third portion positioned between the first and second portions to permit the first and second portions to hybridize with one another.

In another embodiment of this invention, the invention relates to a linear RNA molecule encoded by an RNAi expression cassette capable of forming a dsRNA complex wherein the RNA molecule comprises: (a) a first portion that hybridizes to at least a portion of a mRNA molecule encoded by a gene; and (b) a second portion wherein at least part of the second portion is capable of hybridizing to the first portion and wherein the second portion comprises a transcription termination signal In a further embodiment there is provided a double-stranded RNA complex formed by the RNA transcripts of the RNAi expression cassette, which RNA comprises, (a) a first sequence which, under hybridizing conditions, hybridizes to at least a portion of an mRNA molecule encoded by a gene; and (b) a second sequence which, under hybridizing conditions, hybridizes to the first sequence; and the first and second sequences are part of independent linear RNA molecules.

In another embodiment there is provided a linear RNA molecule encoded by an RNAi expression cassette for forming a double-stranded RNA complex, which RNA comprises, (a) a first sequence which, under hybridizing conditions, hybridizes to at least a portion of an mRNA molecule encoded by a gene; and (b)a second sequence which, under hybridizing conditions, hybridizes to the first sequence; and the complex between sequences one and two produces an artificial hairpin dsRNA.

In another embodiment there is provided a linear RNA molecule encoded by an RNAi expression cassette for forming a double-stranded RNA complex, which RNA molecule comprises, (a) a portion encoding an RNA or protein that enhances the specific activity of dsRNA; and (b) a portion for forming a double-stranded RNA complex, which portion comprises (i) a first sequence which, under hybridizing conditions, hybridizes to at least a portion of an mRNA molecule encoded by the gene; (ii) a second sequence which, under hybridizing conditions, hybridizes to the first sequence; and (iii) a third sequence situated between the first and second sequences so as to permit the first and second sequences to hybridize with each other, whereby, under hybridizing conditions, the portion (b) forms a double-stranded RNA complex upon hybridization between the first and second sequences.

In another embodiment there is provided a double-stranded RNA complex formed by the RNA transcripts of an RNAi expression cassette, which RNA comprises, (a) first sequence which, under hybridizing conditions, hybridizes to at least a portion of an mRNA molecule encoded by a gene; and (b) a second sequence which, under hybridizing conditions, hybridizes to the first sequence; and the second sequence contains at its 3′ end, between the end of the region of complementarity with the first sequence and the polyadenylation signal, a cis-acting hammerhead ribozyme that can cleave within this same region and remove the polyadenylation signal.

This embodiment of the invention utilises a ribozyme to cleave the polyadenylation signal of the RNA molecule, thus retaining the RNA molecule and/or dsRNA in the nucleus.

### (3 .) DETAILED DESCRIPTION OF THE PREFERRED EMBODIMENTS

In one preferred embodiment, the RNAi target organ for inducing RNA interference via rAAV-mediated RNAi expression cassette transfer is the eye, the RNAi target cell is a photoreceptor cell, the RNAi target gene is the Rhodopsin gene, and the RNAi target sequence is a sequence within the Rhodopsin gene that carries a point mutation resulting into an autosomal-dominant disease phenotype.

In another preferred embodiment, the RNAi target organ for inducing RNA interference via rAAV-mediated RNAi expression cassette transfer is the eye, the RNAi target cell is a photoreceptor cell, the RNAi target gene is the Rhodopsin gene, and the RNAi target sequence is any sequence within the Rhodopsin gene. The rAAV vector comprises as gene of interest a Rhodopsin cDNA that is immunized against RNA interference induced by the RNAi expression cassette. Immunization—as meant in this context—can be achieved by introducing silent point mutations (e.g., point mutations within the cDNA that do not change the amino acid sequence of the translated protein) so that the cellular RNAi machinery does not recognize the immunized Rhodopsin version—once the cellular RNAi machinery has been activated by the dsRNA complex encoded by the RNAi expression cassette(s).

In yet another preferred embodiment, the RNAi expression cassette encodes an RNA molecule of the invention capable of forming a dsRNA complex wherein one portion of the RNA is complementary to the RNA of the Rhodopsin gene. In another embodiment, the RNAi expression cassette encodes an RNA molecule of the invention capable of forming a dsRNA complex wherein one portion of the RNA comprises a portion of a sequence of RNA having a Rhodopsin gene sequence.

In certain preferred embodiments, expression of the RNAi target gene is attenuated by at least 2 fold, and more preferably at least 5, 10, 20 or even 50 fold, e.g., relative to the untreated cell or a cell transduced with an RNAi expression construct which does not correspond to the target gene.

In certain preferred embodiments, the target gene is an endogenous gene of the cell. In other embodiments, the target gene is a heterologous gene relative to the genome of the cell, such as a pathogen gene, e.g., a viral gene.

In certain preferred embodiments, the cell is a primate cell, such as a human cell.

### EXAMPLES FOR RNAi EXPRESSION CASSETTES TARGETING THE LUCIFERASE GENE

(1) Example of RNA interference via rAAV-mediated RNAi expression cassette transfer in vivo to the muscle of a mammalian subject.

To demonstrate decrease of gene expression via AAV-mediated RNAi expression cassette transfer in vivo in the muscle of a mammalian subject, we first transduced muscle tissue in vivo with an AAV vector comprising a luciferase expression cassette. Subsequently, we transduced the same muscle tissue with an AAV vector of another serotype comprising an RNAi expression cassette targeting the luciferase gene. Thus, we expect and demonstrate luciferase gene expression to be decreased in mammalian subjects treated with the second AAV vector that comprises the RNAi expression cassette targeting the luciferase gene.

More specifically, AAV virions were prepared and titered as described herein and in prior art [83, 104, 105]. At day 1, 10.exp.11 genomic particles of AAV 2/2 CMV luciferase were injected into the right tibialis muscle of 40 Balb/c mice. At day 28 the following injections were made into the right tibialis muscle of said mice:

Experiment 1: Study Design

| | |
|---|---|
| Group 1 (5 animals) | 10.exp.11 genomic particles of AAV2/5 U6 lucRI- |
| | 1a |
| Group 2 (5 animals) | 10.exp.11 genomic particles of AAV 2/5 RSV |
| | lucRI-1b |
| Group 3 (5 animals) | 10.exp.11 genomic particles of AAV 2/5 U6/U6 |
| | lucRI-2 |
| Group 4 (5 animals) | 10.exp.11 genomic particles of AAV 2/5 U6/U6 |
| | lucRI-3 |
| Group 5 (5 animals) | 10.exp.11 genomic particles of AAV 2/5 U6 lucRI- |
| | 4(sense) and 10.exp.11 genomic particles of |
| | AAV 2/5 U6 lucRI-4(antisense) |
| Group 6 (5 animals) | 10.exp.11 particles of AAV2/5 pol1 lucRI |
| Group 7 (5 animals) | 10.exp.11 particles of AAV2/5 U6 eGFPRI-1a |
| Group 8 (5 animals) | PBS injections |

At day 60, the muscles were harvested, protein extracted and the luciferase activity determined according to manufacturer's instructions (Promega, Madison, Wis. (USA): Luciferase Assay System with Reporter Lysis Buffer #4030). The following results were obtained, expressed as luciferase activity relative to group 8 (PBS injections):

Experiment 1: Results

| | |
|---|---|
| Group 1 | 14% luciferase activity (+/−3% within 95% confidence |
| | interval) |
| Group 2 | 21% luciferase activity (+/−5% within 95% confidence |
| | interval) |
| Group 3 | 33% luciferase acitivty (+/−6% within 95% confidence |
| | interval) |
| Group 4 | 37% luciferase acitivty (+/−5% within 95% confidence |
| | interval) |
| Group 5 | 53% luciferase activity (+/−6% within 95% confidence |
| | interval) |
| Group 6 | 18% luciferase activity (+/−4% within 95% confidence |
| | interval) |
| Group 7 | 102% luciferase activity (+/−4% within 95% confidence |
| | interval) |
| Group 8 | 100% luciferase activity |

Thus, the luciferase-specific RNA interference vectors AAV2/5 U6 lucRI-1a, AAV2/5 U6 lucRI-1b, AAV2/5 U6/U6 lucRI-2, AAV2/5 U6/U6 lucRI-3, AAV2/5 pol1 lucRI and AAV2/5 U6 lucRI-4(sense); AAV2/5 U6 lucRI-4(antisense) were capable of significantly decreasing luciferase expression in muscle of a mammalian subject via AAV-mediated RNAi expression cassette transfer in vivo compared to an untreated control group (group 8). The decrease was specific as no significant decrease of luciferase activity was observed in group 7 (the group with the eGFP-RNAi control vector).

Moreover, the inventors are the first to show that RNA intereference can be achieved using an RNAi expression cassette comprising an RNA Polymerase I promoter.

(2) Example of RNA interference via rAAV-mediated RNAi expression cassette transfer in vivo to the lung of a mammalian subject.

To demonstrate decrease of gene expression via AAV-mediated RNAi expression cassette transfer in vivo in the lung of a mammalian subject, we first transduced lung tissue in vivo with an AAV vector comprising a luciferase expression cassette. Subsequently, we transduced the same lung tissue with an AAV vector of another serotype comprising an RNAi expression cassette targeting the luciferase gene. Thus, we expect and demonstrate luciferase gene expression to be decreased in mammalian subjects treated with the second AAV vector that comprises the RNAi expression cassette targeting the luciferase gene.

More specifically, AAV virions were prepared and titered as described herein and in prior art [83, 104, 105]. At day 1, 5 times 10.exp.11 genomic particles of AAV 2/2 CMV luciferase were administered to the lung of 15 Balb/c mice via nasal instillation. At day 28, the mice received the following administrations via nasal instillation:

Experiment 2: Study Design

| | |
|---|---|
| Group 1 (5 animals) | 10.exp.11 genomic particles of AAV2/5 U6 lucRI- |
| | 1a |
| Group 2 (5 animals) | 10.exp.11 particles of AAV2/5 U6 eGFPRI-1a |
| Group 3 (5 animals) | PBS control |

At day 60, the lungs were harvested, protein extracted and the luciferase activity determined according to manufacturer's instructions (Promega, Madison, Wis. (USA): Luciferase Assay System with Reporter Lysis Buffer #4030). The following results were obtained, expressed as luciferase activity relative to group 3 (PBS instillation):

Experiment 2: Results

| | |
|---|---|
| Group 1 | 27% luciferase activity (+/−5% within 95% confidence interval) |
| Group 2 | 98% luciferase activity (+/−3% within 95% confidence interval) |
| Group 3 | 100% luciferase activity |

Thus, the luciferase-specific RNA interference vector AAV2/5 U6 lucRI-1a was capable of significantly decreasing luciferase expression in lung of a mammalian subject via AAV-mediated RNAi expression cassette transfer in vivo compared to an untreated control group (group 3). The decrease was specific as no significant decrease of luciferase activity was observed in group 2 (the group with the eGFP-RNAi control vector).

(3) Example of RNA interference via rAAV-mediated RNAi expression cassette transfer in vivo to the liver of a mammalian subject.

To demonstrate decrease of gene expression via AAV-mediated RNAi expression cassette transfer in vivo in the liver of a mammalian subject, we first transduced liver tissue in vivo with an AAV vector comprising a luciferase expression cassette. Subsequently, we transduced the same liver tissue with an AAV vector of another serotype comprising an RNAi expression cassette targeting the luciferase gene. Thus, we expect and demonstrate luciferase gene expression to be decreased in mammalian subjects treated with the second AAV vector that comprises the RNAi expression cassette targeting the luciferase gene.

More specifically, AAV virions were prepared and titered as described herein and in prior art [83, 104, 105]. At day 1, 10.exp.12 genomic particles of AAV 2/2 CMV luciferase were administered to the liver of 15 Balb/c mice via portal vein injection. At day 28, the mice received the following administrations via portal vein injection:(

Experiment 3: Study Design

| | |
|---|---|
| Group 1 (5 animals) | 10.exp.12 genomic particles of AAV2/5 U6 lucRI- |
| | 1a |
| Group 1 (5 animals) | 10.exp.12 particles of AAV2/5 U6 eGFPRI-1a |
| Group 3 (5 animals) | PBS control |

At day 60, the livers were harvested, protein extracted and the luciferase activity determined according to manufacturer's instructions (Promega, Madison, Wis. (USA): Luciferase Assay System with Reporter Lysis Buffer #4030)). The following results were obtained, expressed as luciferase activity relative to group 3 (PBS injection):

Experiment 3

| | |
|---|---|
| Group 1 | 48% luciferase activity (+/−9% within 95% confidence interval) |
| Group 2 | 99% luciferase activity (+/−5% within 95% confidence interval) |
| Group 3 | 00% luciferase activity |

Thus, the luciferase-specific RNA interference vector AAV2/5 U6 lucRI-1a was capable of significantly decreasing luciferase expression in liver of a mammalian subject via AAV-mediated RNAi expression cassette transfer in vivo compared to an untreated control group (group 3). The decrease was specific as no significant decrease of luciferase activity was observed in group 2 (the group with the eGFP-RNAi control vector).

(4) Example of RNA interference via rAAV-mediated RNAi expression cassette transfer in vivo to the brain of a mammalian subject.

To demonstrate decrease of gene expression via AAV-mediated RNAi expression cassette transfer in vivo in the brain of a mammalian subject, we first transduced the brain in vivo with an AAV vector comprising a luciferase expression cassette. Subsequently, we transduced the same brain with an AAV vector of another serotype comprising an RNAi expression cassette targeting the luciferase gene. Thus, we expect and demonstrate luciferase gene expression to be decreased in mammalian subjects treated with the second AAV vector that comprises the RNAi expression cassette targeting the luciferase gene.

More specifically, AAV virions were prepared and titered as described herein and in prior art [83, 104, 105]. At day 1, 10.exp.10 genomic particles of AAV 2/2 CMV luciferase were administered to the brain of 15 Balb/c mice via intracranial injections. At day 28, the mice received the following administrations via intracranial injections:

Experiment 4: Study Design

| | |
|---|---|
| Group 1 (5 animals) | 10.exp.12 genomic particles of AAV2/5 U6 lucRI- |
| | 1a |
| Group 2 (5 animals) | 10.exp.12 particles of AAV2/5 U6 eGFPRI-1a |
| Group 3 (5 animals) | PBS control |

At day 60, the brains were harvested, protein extracted and the luciferase activity determined according to manufacturer's instructions (Promega, Madison, Wis. (USA): Luciferase Assay System with Reporter Lysis Buffer #4030). The following results were obtained, expressed as luciferase activity relative to group 3 (PBS injection):

Experiment 4: Results

| | |
|---|---|
| Group 1 | 21% luciferase activity (+/−5% within 95% confidence interval) |
| Group 2 | 101% luciferase activity (+/−2% within 95% confidence interval) |
| Group 3 | 100% luciferase activity |

Thus, the luciferase-specific RNA interference vector AAV2/5 U6 lucRI-1a was capable of significantly decreasing luciferase expression in brain of a mammalian subject via AAV-mediated RNAi expression cassette transfer in vivo compared to an untreated control group (group 3). The decrease was specific as no significant decrease of luciferase activity was observed in group 2 (the group with the eGFP-RNAi control vector).

(5) Example of RNA interference via rAAV-mediated RNAi expression cassette transfer in vivo to the eye of a mammalian subject.

To demonstrate decrease of gene expression via AAV-mediated RNAi expression cassette transfer in vivo in the eye of a mammalian subject, we first transduced the eye in vivo with an AAV vector comprising a luciferase expression cassette. Subsequently, we transduced the same eye with an AAV vector of another serotype comprising an RNAi expression cassette targeting the luciferase gene. Thus, we expect and demonstrate luciferase gene expression to be decreased in mammalian subjects treated with the second AAV vector that comprises the RNAi expression cassette targeting the luciferase gene.

More specifically, AAV virions were prepared and titered as described herein and in prior art [83, 104, 105]. At day 1, 5.exp.9 genomic particles of AAV 2/2 CMV luciferase were administered to the right eye of 15 Balb/c mice via intravitreal injections. At day 28, the mice received the following administrations via intravitreal injections into the same eye:(

Experiment 5: Study Design

| | |
|---|---|
| Group 1 (5 animals) | 5.exp.9 genomic particles of AAV2/5 U6 lucRI-1a |
| Group 2 (5 animals) | 5.exp.9 particles of AAV2/5 U6 eGFPRI-1a |
| Group 3 (5 animals) | PBS control |

At day 60, the right eyes were harvested, protein extracted and the luciferase activity determined according to manufacturer's instructions (Promega, Madison, Wis. (USA): Luciferase Assay System with Reporter Lysis Buffer #4030). The following results were obtained, expressed as luciferase activity relative to group 3 (PBS injection):

Experiment 5: Results

| | |
|---|---|
| Group 1 | 11% luciferase activity (+/−3% within 95% confidence interval) |
| Group 2 | 99% luciferase activity (+/−2% within 95% confidence interval) |
| Group 3 | 100% luciferase activity |

Thus, the luciferase-specific RNA interference vector AAV2/5 U6 lucRI-1a was capable of significantly decreasing luciferase expression in the eye of a mammalian subject via AAV-mediated RNAi expression cassette transfer in vivo compared to an untreated control group (group 3). The decrease was specific as no significant decrease of luciferase activity was observed in group 2 (the group with the eGFP-RNAi control vector).

(6) Example of RNA interference via rAAV-mediated RNAi expression cassette transfer in vivo to the photoreceptor cells within the eye of a mammalian subject.

To demonstrate decrease of endogenous gene expression via AAV-mediated RNAi expression cassette transfer in vivo in photoreceptor cells of the eye of a mammalian subject, we transduced the eye of a GFP transgenic mammal with an AAV2 vector comprising an RNAi expression cassette targeting the GFP gene. Thus, we expect and demonstrate endogenous gene expression to be decreased in mammalian subjects treated with an AAV vector that comprises an RNAi expression cassette targeting an endogenous gene.

More specifically, AAV2/2 virions were prepared and titered as described herein and in prior art [83, 104, 105]. At day 1, 5.exp.9 genomic particles of AAV 2/2 U6 eGFPRI-1a were administered to the right eye of 10 Balb/c mice via intravitreal injections. The left eyes of the same animals received 1, 5.exp.9 genomic particles of AAV 2/2 U6 luciferase-1a via intravitreal injections and functioned as a negative control.

At day 28, the eyes were harvested, and cryosections of left and right eyes were made as described in prior art. Subsequently, the percentage of GFP-negative photoreceptor cells was determined by fluorescence microscopy. We analyzed a total of 2,587 photoreceptor cells from the left eye cryosections close to the site of vector administration and found 2,103 GFP negative photoreceptor cells, a reduction of gene expression in ˜81% of the target photoreceptor cells. For comparison: We analyzed 1,908 photoreceptor cells from the right eye cryosections close to the injection site and found a total of 87 GFP negative photoreceptor cells.

Thus, the GFP-specific RNA interference vector AAV2/2 U6 eGFPRI-1a was capable of significantly decreasing GFP expression in the eye of a GFP transgenic mammalian subject via AAV-mediated RNAi expression cassette transfer in vivo. The decrease was specific as no significant decrease of GFP activity was observed in control eyes. Generally, AAV-mediated RNAi expression cassette transfer to photoreceptor cells in vivo can be used to decrease expression of endogenous genes.

The artisan will be able to reconstruct all the plasmid constructs described herein from the sequence information provided. For example, the artisan might choose to order overlapping oligonucleotides according to the sequence information provided to clone any construct s/he desires to reproduce. Alternatively, commercial cloning services are available that will reproduce any construct based on sequence information provided (Qiagen, Hilden (Germany); Invitrogen, Carlsbad, Calif. (USA)).

Although the present invention has been described with reference to specific embodiments, numerous modifications and variations can be made and still the result will come within the scope of the invention. No limitation with respect to the specific embodiments disclosed herein is intended or should be inferred. Those skilled in the art will readily appreciate that the specific experiments detailed are only illustrative of the invention as described more fully in the claims. Additionally, throughout this application, various publications are cited. The disclosure of these publications is hereby incorporated by reference into this application to describe more fully the state of the art to which this invention pertains. The examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

Sequence of AAV2/2 CMV luciferase and AAV2/5 CMV luciferase
5′-agcgcccaatacgcaaaccgcctctccccgcgcgttggccgattcattaatg
cagctggcacgacaggtttcccgactggaaagcgggcagtgagcgcaacg-
caattaatgtgagttagctcactcattaggcaccccaggctttacactttat-
gcttccggctcgtatgttgtgtggaattgtgagcggataacaatttca-
cacaggaaacagctatgaccatgattacgccagatttaattaaggct-
gcgcgctcgctcgctcactgaggccgcccgggcaaagcccgggcgtcgggc-
gacctttggtcgcccggcctcagtgagcgagcgagcgcgcaga-
gagggagtggccaactccatcactaggggttccttgtagttaatgattaacc-
cgccatgctacttatctacgtagccatgctctaggaagatcggaattcgccct-
taagctagctagttattaatagtaatcaattacggggtcattagttcatagcc-
catatatggagttccgcgttacataacttacggtaaatggcccgcctggctgac-
cgcccaacgacccccgcccattgacgtcaataatgacgtatgttcccatag-
taacgccaatagggactttccattgacgtcaatgggtggagtatttacg-
gtaaactgcccacttggcagtacatcaagtgtatcatatgccaagtacgccccc-
tattgacgtcaatgacggtaaatggcccgcctggcattatgcccagtacatgac-
cttatgggactttcctacttggcagtacatctacgtattagtcatcgctattac-
catggtgatgcggttttggcagtacatcaatgggcgtggatagcggtttgact-
cacggggatttccaagtctccaccccattgacgtcaatgggagtttgttttg-
gcaccaaaatcaacgggactttccaaaatgtcgtaacaactccgccc-
cattgacgcaaatgggcggtaggcgtgtacggtgggaggtctatataagca-
gagctggtttagtgaaccgtcagatcctgcagaagttggtcgtgag-
gcactgggcaggtaagtatcaaggttacaagacaggtttaaggagaccaata-
gaaactgggcttgtcgagacagagaagactcttgcgtttctgataggcacc-
tattggtcttactgacatccactttgcctttctctccacaggtgtccaggcggc-
cgccatggaagacgccaaaaacataaagaaaggcccggcgccattctatc-
cgctggaagatggaaccgctggagagcaactgcataaggctatgaagagat-
acgccctggttcctggaacaattgcttttacagatgcacatatcgaggtgga-
catcacttacgctgagtacttcgaaatgtccgttcggttggcagaagctat-
gaaacgatatgggctgaatacaaatcacagaatcgtcgtatgcagt-
gaaaactctcttcaattctttatgccggtgttgggcgcgttatttatcg-
gagttgcagttgcgcccgcgaacgacatttataatgaacgtgaattgct-
caacagtatgggcatttcgcagcctaccgtggtgttcgtttc-
caaaaaggggttgcaaaaaattttgaacgtgcaaaaaaagctcccaatcatc-
caaaaaattattatcatggattctaaaacggattaccagggatttcagtcgatg-
tacacgttcgtcacatctcatctacctcccggttttaatgaatacgattttgtgcca-
gagtccttcgatagggacaagacaattgcactgatcatgaactcctctggatc-
tactggtctgcctaaaggtgtcgctctgcctcatagaactgcctgcgtga-
gattctcgcatgccagagatcctatttttggcaatcaaatcattccggatactgc-
gattttaagtgttgttccattccatcacggttttggaatgtttactacactcg-
gatatttgatatgtggatttcgagtcgtcttaatgtatagatttgaagaagagct-
gtttctgaggagccttcaggattacaagattcaaagtgcgctgctggtgccaac-
cctattctccttcttcgccaaaagcactctgattgacaaatacgatttatctaatt-
tacacgaaattgcttctggtggcgctcccctctctaaggaagtcggggaagcg-
gttgccaagaggttccatctgccaggtatcaggcaaggatatgggctcactga-
gactacatcagctattctgattacacccgagggggatgataaaccgggcgcg-
gtcggtaaagttgttccattttttgaagcgaaggttgtggatctggatac-
cgggaaaacgctgggcgttaatcaaagaggcgaactgtgtgtgagaggtcc-
tatgattatgtccggttatgtaaacaatccggaagcgaccaacgccttgattga-
caaggatggatggctacattctggagacatagcttactgggacgaagacgaa-
cacttcttcatcgttgaccgcctgaagtctctgattaagtacaaaggc-
tatcaggtggctcccgctgaattggaatccatcttgctccaacaccccaa-
catcttcgacgcaggtgtcgcaggtcttcccgacgatgacgccggtgaacttc-
ccgccgccgttgttgttttggagcacggaaagacgatgacggaaaaaga-
gatcgtggattacgtcgccagtcaagtaacaaccgcgaaaaagttgcgcg-
gaggagttgtgtttgtggacgaagtaccgaaaggtcttaccggaaaactc-
gacgcaagaaaaatcagagagatcctcataaaggccaagaagggcggaaa-
gatcgccgtgtaataagcttggatccaatcaacctctggattacaaaatttgt-
gaaagattgactggtattcttaactatgttgctccttttacgctatgtggat-
acgctgctttaatgcctttgtatcatgctattgcttcccgtatggctttcattttctc-
ctccttgtataaatcctggttgctgtctctttatgaggagttgtggcc-
cgttgtcaggcaacgtggcgtggtgtgcactgtgtttgctgacgcaacccc-
cactggttggggcattgccaccacctgtcagctcctttccgggactttcgctttc-
cccctccctattgccacggcggaactcatcgccgcctgccttgcccgctgctg-
gacaggggctcggctgttgggcactgacaattccgtggt-
gttgtcggggaagctgacgtcctttccatggctgctcgcctgtgttgccacctg-
gattctgcgcgggacgtccttctgctacgtcccttcggccctcaatccagcg-
gaccttccttcccgcggcctgctgccggctctgcggcctcttccgcgtcttcga-
gatctgcctcgactgtgccttctagttgccagccatctgttgtttgcccctcccc-
cgtgccttccttgaccctggaaggtgccactcccactgtcctttcctaataaaat-
gaggaaattgcatcgcattgtctgagtaggtgtcattc-
tattctggggggtggggtggggcaggacagcaagggggaggattgggaa-
gacaatagcaggcatgctggggactcgagttaagggcgaattcccgattag-
gatcttcctagagcatggctacgtagataagtagcatggcgggttaatcat-
taactacaaggaacccctagtgatggagttggccactccctctct-
gcgcgctcgctcgctcactgaggccgggcgaccaaaggtcgcccgacgcc-
cgggctttgcccgggcggcctcagtgagcgagcgagcgcgcagccttaat-
taacctaattcactggccgtcgttttacaacgtcgtgactgggaaaaccctg-
gcgttacccaacttaatcgccttgcagcacatccccctttcgccagctggcg-
taatagcgaagaggcccgcaccgatcgcccttcccaacagttgcgcagcct-
gaatggcgaatgggacgcgccctgtagcggcgcattaagcgcggcgggt-
gtggtggttacgcgcagcgtgaccgctacacttgccagcgccctagcgcc-
cgctcctttcgctttcttcccttcctttctcgccacgttcgccggctttccccgt-
caagctctaaatcgggggctccctttagggttccgatttagtgctttacggcac-
ctcgaccccaaaaaacttgattagggtgatggttcacgtagtgggccatcgcc-
ccgatagacggtttttcgccctttgacgctggagttcacgttcctcaatagtg-
gactcttgttccaaactggaacaacactcaaccctatctcggtctattcttttgatt-
tataagggatttttccgatttcggcctattggttaaaaaatgagctgatttaa-
caaaaatttaacgcgaattttaacaaaatattaacgtttataatttcaggtg-
gcatctttcggggaaatgtgcgcggaacccctatttgtttatttttctaaata-
cattcaaatatgtatccgctcatgagacaataaccctgataaat-
gcttcaataatattgaaaaaggaagagtatgagtattcaacatttccgtgtcgcc-
cttattcccttttttgcggcattttgccttcctgtttttgctcacccagaaacgctg-
gtgaaagtaaaagatgctgaagatcagttgggtgcacgagtgggttacatc-
gaactggatctcaatagtggtaagatccttgagagttttcgccccgaa-
gaacgttttccaatgatgagcacttttaaagttctgctatgtggcgcggtattatc-
ccgtattgacgccgggcaagagcaactcggtcgccgcatacactattctca-
gaatgacttggttgagtactcaccagtcacagaaaagcatcttacggatg-
gcatgacagtaagagaattatgcagtgctgccataaccatgagtgataacact-
gcggccaacttacttctgacaacgatcggaggaccgaaggagctaac-
cgcttttttgcacaacatgggggatcatgtaactcgccttgatcgttgggaac-
cggagctgaatgaagccataccaaacgacgagcgtgacaccacgatgcctg-
tagtaatggtaacaacgttgcgcaaactattaactggcgaactacttactc-
tagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′
Sequence of AAV2/5 U6 lucRI-1a
5′- agcgcccaatacgcaaaccgc-
ctctccccgcgcgttggccgattcattaatgcagctggcacgacaggtttccc-
gactggaaagcgggcagtgagcgcaacgcaattaatgtgagttagctcact-
cattaggcaccccaggctttacactttatgcttccggctcgtatgttgtgtg-
gaattgtgagcggataacaatttcacacaggaaacagctatgaccatgat-
tacgccagatttaattaaggctgcgcgctcgctcgctcactgaggccgcc-
cgggcaaagcccgggcgtcgggcgacctttggtcgcccggcctcagtgagc-
gagcgagcgcgcagagagggagtggccaactccatcactaggggttccttg-
tagttaatgattaacccgccatgctacttatctacgtagccatgctctaggaa-
gatcggaattcgcccttaagctagcccccagtggaaa-
gacgcgcaggcaaaacgcaccacgtgacggagcgtgaccgcgcgccgagc-
ccaaggtcgggcaggaagagggcctatttcccatgattccttcatatttgcatat-
acgatacaaggctgttagagagataattagaattaatttgactgtaaacacaaa-
gatattagtacaaaatacgtgacgtagaaag-
taataatttcttgggtagtttgcagttttaaaattatgttttaaaatggactat-
catatgcttaccgtaacttgaaagtatttcgatttcttggctttatatatcttgtg-
gaaaggacgaaacacccttacgctgagtacttcgattcaagagatcgaag-
tactcagcgtaagtttttctcgagttaagggcgaattcccgattaggatcttccta-
gagcatggctacgtagataagtagcatggcgggttaatcattaactacaag-
gaacccctagtgatggagttggccactccctctctgcgcgctcgctcgctcact-
gaggccgggcgaccaaaggtcgcccgacgcccgggctttgcccgggcggc-
ctcagtgagcgagcgagcgcgcagccttaattaacctaattcactggc-
cgtcgttttacaacgtcgtgactgggaaaaccctggcgttacccaact-
taatcgccttgcagcacatccccctttcgccagctggcgtaatagcgaagag-
gcccgcaccgatcgcccttcccaacagttgcgcagcctgaatggc-
gaatgggacgcgccctgtagcggcgcattaagcgcggcgggtgtggtggt-
tacgcgcagcgtgaccgctacacttgccagcgccctagcgcccgctc-
ctttcgctttcttcccttcctttctcgccacgttcgccggctttccccgtcaagctc-
taaatcgggggctccctttagggttccgatttagtgctttacggcacctcgaccc-
caaaaaacttgattagggtgatggttcacgtagtgggccatcgccccgata-
gacggtttttcgccctttgacgctggagttcacgttcctcaatagtg-
gactcttgttccaaactggaacaacactcaaccctatctcggtctattcttttgatt-
tataagggatttttccgatttcggcctattggttaaaaaatgagctgatttaa-
caaaaatttaacgcgaattttaacaaaatattaacgtttataatttcaggtg-
gcatctttcggggaaatgtgcgcggaacccctatttgtttatttttctaaata-
cattcaaatatgtatccgctcatgagacaataaccctgataaat-
gcttcaataatattgaaaaaggaagagtatgagtattcaacatttccgtgtcgcc-
cttattcccttttttgcggcattttgccttcctgtttttgctcacccagaaacgctg-
gtgaaagtaaaagatgctgaagatcagttgggtgcacgagtgggttacatc-
gaactggatctcaatagtggtaagatccttgagagttttcgccccgaa-
gaacgttttccaatgatgagcacttttaaagttctgctatgtggcgcggtattatc-
ccgtattgacgccgggcaagagcaactcggtcgccgcatacactattctca-
gaatgacttggttgagtactcaccagtcacagaaaagcatcttacggatg-
gcatgacagtaagagaattatgcagtgctgccataaccatgagtgataacact-
gcggccaacttacttctgacaacgatcggaggaccgaaggagctaac-
cgcttttttgcacaacatgggggatcatgtaactcgccttgatcgttgggaac-
cggagctgaatgaagccataccaaacgacgagcgtgacaccacgatgcctg-
tagtaatggtaacaacgttgcgcaaactattaactggcgaactacttactc-
tagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′
Sequence of AAV2/5 CMV lucRI-1b
5′- agcgcccaatacgcaaac-
cgcctctccccgcgcgttggccgattcattaatgcagctggcacgacaggtttc-
ccgactggaaagcgggcagtgagcgcaacgcaattaatgtgagttagct-
cactcattaggcaccccaggctttacactttatgcttccggctcgtatgttgtgtg-
gaattgtgagcggataacaatttcacacaggaaacagctatgaccatgat-
tacgccagatttaattaaggctgcgcgctcgctcgctcactgaggccgcc-
cgggcaaagcccgggcgtcgggcgacctttggtcgcccggcctcagtgagc-
gagcgagcgcgcagagagggagtggccaactccatcactaggggttccttg-
tagttaatgattaacccgccatgctacttatctacgtagccatgctctaggaa-
gatcggaattcgcccttaagctagctagttattaatagtaatcaattacggggt-
cattagttcatagcccatatatggagttccgcgttacataacttacggtaaatg-
gcccgcctggctgaccgcccaacgacccccgcccattgacgtcaataatgacg-
tatgttcccatagtaacgccaatagggactttccattgacgtcaatgggtggag-
tatttacggtaaactgcccacttggcagtacatcaagtgtatcatatgccaag-
tacgccccctattgacgtcaatgacggtaaatggcccgcctggcattatgc-
ccagtacatgaccttatgggactttcctacttggcagtacatctacgtattagt-
catcgctattaccatggtgatgcggttttggcagtacatcaatgggcgtg-
gatagcggtttgactcacggggatttccaagtctccaccccattgacgt-
caatgggagtttgttttggcaccaaaatcaacgggactttccaaaatgtcgtaa-
caactccgccccattgacgcaaatgggcggtaggcgtgtacggtgggaggtc-
tatataagcagagctggtttagtgaaccgtcttacgctgagtacttcgattcaa-
gagatcgaagtactcagcgtaaggctagcacacaaaaaaccaacacaca-
gatctaatgaaaataaagatcttttactcgagttaagggcgaattcccgattag-
gatcttcctagagcatggctacgtagataagtagcatggcgggttaatcat-
taactacaaggaacccctagtgatggagttggccactccctctct-
gcgcgctcgctcgctcactgaggccgggcgaccaaaggtcgcccgacgcc-
cgggctttgcccgggcggcctcagtgagcgagcgagcgcgcagccttaat-
taacctaattcactggccgtcgttttacaacgtcgtgactgggaaaaccctg-
gcgttacccaacttaatcgccttgcagcacatccccctttcgccagctggcg-
taatagcgaagaggcccgcaccgatcgcccttcccaacagttgcgcagcct-
gaatggcgaatgggacgcgccctgtagcggcgcattaagcgcggcgggt-
gtggtggttacgcgcagcgtgaccgctacacttgccagcgccctagcgcc-
cgctcctttcgctttcttcccttcctttctcgccacgttcgccggctttccccgt-
caagctctaaatcgggggctccctttagggttccgatttagtgctttacggcac-
ctcgaccccaaaaaacttgattagggtgatggttcacgtagtgggccatcgcc-
ccgatagacggtttttcgccctttgacgctggagttcacgttcctcaatagtg-
gactcttgttccaaactggaacaacactcaaccctatctcggtctattcttttgatt-
tataagggatttttccgatttcggcctattggttaaaaaatgagctgatttaa-
caaaaatttaacgcgaattttaacaaaatattaacgtttataatttcaggtg-
gcatctttcggggaaatgtgcgcggaacccctatttgtttatttttctaaata-
cattcaaatatgtatccgctcatgagacaataaccctgataaat-
gcttcaataatattgaaaaaggaagagtatgagtattcaacatttccgtgtcgcc-
cttattcccttttttgcggcattttgccttcctgtttttgctcacccagaaacgctg-
gtgaaagtaaaagatgctgaagatcagttgggtgcacgagtgggttacatc-
gaactggatctcaatagtggtaagatccttgagagttttcgccccgaa-
gaacgttttccaatgatgagcacttttaaagttctgctatgtggcgcggtattatc-
ccgtattgacgccgggcaagagcaactcggtcgccgcatacactattctca-
gaatgacttggttgagtactcaccagtcacagaaaagcatcttacggatg-
gcatgacagtaagagaattatgcagtgctgccataaccatgagtgataacact-
gcggccaacttacttctgacaacgatcggaggaccgaaggagctaac-
cgcttttttgcacaacatgggggatcatgtaactcgccttgatcgttgggaac-
cggagctgaatgaagccataccaaacgacgagcgtgacaccacgatgcctg-
tagtaatggtaacaacgttgcgcaaactattaactggcgaactacttactc-
tagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′
Sequence of AAV2/5 U6/U6 lucRI-2
5′-agcgcccaatacgcaaaccgcctctccccgcgcgttggccgattcattaatg
cagctggcacgacaggtttcccgactggaaagcgggcagtgagcgcaacg-
caattaatgtgagttagctcactcattaggcaccccaggctttacactttat-
gcttccggctcgtatgttgtgtggaattgtgagcggataacaatttca-
cacaggaaacagctatgaccatgattacgccagatttaattaaggct-
gcgcgctcgctcgctcactgaggccgcccgggcaaagcccgggcgtcgggc-
gacctttggtcgcccggcctcagtgagcgagcgagcgcgcaga-
gagggagtggccaactccatcactaggggttccttgtagttaatgattaacc-
cgccatgctacttatctacgtagccatgctctaggaagatcggaattcgccct-
taagctagcccccagtggaaagacgcgcaggcaaaacgcaccacgtgacg-
gagcgtgaccgcgcgccgagcccaaggtcgggcaggaagagggcctatttc-
ccatgattccttcatatttgcatatacgatacaaggctgttagagagataatta-
gaattaatttgactgtaaacacaaagatattagtacaaaatacgtgacgta-
gaaagtaataatttcttgggtagtttgcagttttaaaattatgttttaaaatggac-
tatcatatgcttaccgtaacttgaaagtatttcgatttcttggctt-
tatatatcttgtggaaaggacgaaacacccttacgctgagtacttcgattttcc-
ccagtggaaagacgcgcaggcaaaacgcaccacgtgacggagcgtgac-
cgcgcgccgagcccaaggtcgggcaggaagagggcctatttcccatgattc-
cttcatatttgcatatacgatacaaggctgttagagagataattagaat-
taatttgactgtaaacacaaagatattagtacaaaatacgtgacgtagaaag-
taataatttcttgggtagtttgcagttttaaaattatgttttaaaatggactat-
catatgcttaccgtaacttgaaagtatttcgatttcttggctttatatatcttgtg-
gaaaggacgaaacacctcgaagtactcagcgtaagtttttctcgagt-
taagggcgaattcccgattaggatcttcctagagcatggctacgtagataag-
tagcatggcgggttaatcattaactacaaggaacccctagtgatggagttggc-
cactccctctctgcgcgctcgctcgctcactgaggccgggcgaccaaag-
gtcgcccgacgcccgggctttgcccgggcggcctcagtgagcgagc-
gagcgcgcagccttaattaacctaattcactggccgtcgttttacaacgtcgt-
gactgggaaaaccctggcgttacccaacttaatcgccttgcagcacatcccc-
ctttcgccagctggcgtaatagcgaagaggcccgcaccgatcgcccttcc-
caacagttgcgcagcctgaatggcgaatgggacgcgccctgtagcggcg-
cattaagcgcggcgggtgtggtggttacgcgcagcgtgaccgcta-
cacttgccagcgccctagcgcccgctcctttcgctttcttcccttcctttctcgc-
cacgttcgccggctttccccgtcaagctctaaatcgggggctccctttagggttc-
cgatttagtgctttacggcacctcgaccccaaaaaacttgattagggtgatg-
gttcacgtagtgggccatcgccccgatagacggtttttcgccctttgacgctg-
gagttcacgttcctcaatagtggactcttgttccaaactggaacaacactcaacc-
ctatctcggtctattcttttgatttataagggatttttccgatttcggcctattggt-
taaaaaatgagctgatttaacaaaaatttaacgcgaattttaacaaaatat-
taacgtttataatttcaggtggcatctttcggggaaatgtgcgcggaacccc-
tatttgtttatttttctaaatacattcaaatatgtatccgctcatgagacaataacc-
ctgataaatgcttcaataatattgaaaaaggaagagtatgagtattcaacatttc-
cgtgtcgcccttattcccttttttgcggcattttgccttcctgtttttgctcaccca-
gaaacgctggtgaaagtaaaagatgctgaagatcagttgggtgcac-
gagtgggttacatcgaactggatctcaatagtggtaagatccttga-
gagttttcgccccgaagaacgttttccaatgatgagcacttttaaagttctgctat-
gtggcgcggtattatcccgtattgacgccgggcaagagcaactcggtcgccg-
catacactattctcagaatgacttggttgagtactcaccagtcacagaaaag-
catcttacggatggcatgacagtaagagaattatgcagtgctgccataaccat-
gagtgataacactgcggccaacttacttctgacaacgatcggaggaccgaag-
gagctaaccgcttttttgcacaacatgggggatcatgtaactcgc-
cttgatcgttgggaaccggagctgaatgaagccataccaaacgacgagcgt-
gacaccacgatgcctgtagtaatggtaacaacgttgcgcaaactattaactg-
gcgaactacttactctagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′
Sequence of AAV2/5 U6/U6 lucRIU6-3
5′-agcgcccaatacgcaaaccgcctctccccgcgcgttggccgattcattaatg
cagctggcacgacaggtttcccgactggaaagcgggcagtgagcgcaacg-
caattaatgtgagttagctcactcattaggcaccccaggctttacactttat-
gcttccggctcgtatgttgtgtggaattgtgagcggataacaatttca-
cacaggaaacagctatgaccatgattacgccagatttaattaaggct-
gcgcgctcgctcgctcactgaggccgcccgggcaaagcccgggcgtcgggc-
gacctttggtcgcccggcctcagtgagcgagcgagcgcgcaga-
gagggagtggccaactccatcactaggggttccttgtagttaatgattaacc-
cgccatgctacttatctacgtagccatgctctaggaagatcggaattcgccct-
taagctagcccccagtggaaagacgcgcaggcaaaacgcaccacgtgacg-
gagcgtgaccgcgcgccgagcccaaggtcgggcaggaagagggcctatttc-
ccatgattccttcatatttgcatatacgatacaaggctgttagagagataatta-
gaattaatttgactgtaaacacaaagatattagtacaaaatacgtgacgta-
gaaagtaataatttcttgggtagtttgcagttttaaaattatgttttaaaatggac-
tatcatatgcttaccgtaacttgaaagtatttcgatttcttggctt-
tatatatcttgtggaaaggacgaaacacctttttcttacgctgagtacttc-
gatttttggtgtttcgtcctttccacaagatatataaagccaagaaatcgaaat-
actttcaagttacggtaagcatatgatagtccattttaaaacataattttaaaact-
gcaaactacccaagaaattattactttctacgtcacgtattttgtac-
taatatctttgtgtttacagtcaaattaattctaattatctctctaacagccttg-
tatcgtatatgcaaatatgaaggaatcatgggaaataggccctcttcctgccc-
gaccttgggctcggcgcgcggtcacgctccgtcacgtggtgcgttttgcct-
gcgcgtctttccactggggctcgagttaagggcgaattcccgattaggatcttc-
ctagagcatggctacgtagataagtagcatggcgggttaatcattaacta-
caaggaacccctagtgatggagttggccactccctctctgcgcgctcgctcgct-
cactgaggccgggcgaccaaaggtcgcccgacgcccgggctttgcc-
cgggcggcctcagtgagcgagcgagcgcgcagccttaattaacc-
taattcactggccgtcgttttacaacgtcgtgactgggaaaaccctggcgttac-
ccaacttaatcgccttgcagcacatccccctttcgccagctggcgtaatagc-
gaagaggcccgcaccgatcgcccttcccaacagttgcgcagcctgaatggc-
gaatgggacgcgccctgtagcggcgcattaagcgcggcgggtgtggtggt-
tacgcgcagcgtgaccgctacacttgccagcgccctagcgcccgctc-
ctttcgctttcttcccttcctttctcgccacgttcgccggctttccccgtcaagctc-
taaatcgggggctccctttagggttccgatttagtgctttacggcacctcgaccc-
caaaaaacttgattagggtgatggttcacgtagtgggccatcgccccgata-
gacggtttttcgccctttgacgctggagttcacgttcctcaatagtg-
gactcttgttccaaactggaacaacactcaaccctatctcggtctattcttttgatt-
tataagggatttttccgatttcggcctattggttaaaaaatgagctgatttaa-
caaaaatttaacgcgaattttaacaaaatattaacgtttataatttcaggtg-
gcatctttcggggaaatgtgcgcggaacccctatttgtttatttttctaaata-
cattcaaatatgtatccgctcatgagacaataaccctgataaat-
gcttcaataatattgaaaaaggaagagtatgagtattcaacatttccgtgtcgcc-
cttattcccttttttgcggcattttgccttcctgtttttgctcacccagaaacgctg-
gtgaaagtaaaagatgctgaagatcagttgggtgcacgagtgggttacatc-
gaactggatctcaatagtggtaagatccttgagagttttcgccccgaa-
gaacgttttccaatgatgagcacttttaaagttctgctatgtggcgcggtattatc-
ccgtattgacgccgggcaagagcaactcggtcgccgcatacactattctca-
gaatgacttggttgagtactcaccagtcacagaaaagcatcttacggatg-
gcatgacagtaagagaattatgcagtgctgccataaccatgagtgataacact-
gcggccaacttacttctgacaacgatcggaggaccgaaggagctaac-
cgcttttttgcacaacatgggggatcatgtaactcgccttgatcgttgggaac-
cggagctgaatgaagccataccaaacgacgagcgtgacaccacgatgcctg-
tagtaatggtaacaacgttgcgcaaactattaactggcgaactacttactc-
tagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′
Sequence of AAV2/5 U6 lucRI-4 (sense)
5′-agcgcccaatacgcaaaccgcctctccccgcgcgttggccgattcattaatg
cagctggcacgacaggtttcccgactggaaagcgggcagtgagcgcaacg-
caattaatgtgagttagctcactcattaggcaccccaggctttacactttat-
gcttccggctcgtatgttgtgtggaattgtgagcggataacaatttca-
cacaggaaacagctatgaccatgattacgccagatttaattaaggct-
gcgcgctcgctcgctcactgaggccgcccgggcaaagcccgggcgtcgggc-
gacctttggtcgcccggcctcagtgagcgagcgagcgcgcaga-
gagggagtggccaactccatcactaggggttccttgtagttaatgattaacc-
cgccatgctacttatctacgtagccatgctctaggaagatcggaattcgccct-
taagctagcccccagtggaaagacgcgcaggcaaaacgcaccacgtgacg-
gagcgtgaccgcgcgccgagcccaaggtcgggcaggaagagggcctatttc-
ccatgattccttcatatttgcatatacgatacaaggctgttagagagataatta-
gaattaatttgactgtaaacacaaagatattagtacaaaatacgtgacgta-
gaaagtaataatttcttgggtagtttgcagttttaaaattatgttttaaaatggac-
tatcatatgcttaccgtaacttgaaagtatttcgatttcttggctt-
tatatatcttgtggaaaggacgaaacacccttacgctgagtacttcgattttctc-
gagttaagggcgaattcccgattaggatcttcctagagcatggctacgta-
gataagtagcatggcgggttaatcattaactacaaggaacccctagtgatg-
gagttggccactccctctctgcgcgctcgctcgctcactgaggccgggcgac-
caaaggtcgcccgacgcccgggctttgcccgggcggcctcagtgagcgagc-
gagcgcgcagccttaattaacctaattcactggccgtcgttttacaacgtcgt-
gactgggaaaaccctggcgttacccaacttaatcgccttgcagcacatcccc-
ctttcgccagctggcgtaatagcgaagaggcccgcaccgatcgcccttcc-
caacagttgcgcagcctgaatggcgaatgggacgcgccctgtagcggcg-
cattaagcgcggcgggtgtggtggttacgcgcagcgtgaccgcta-
cacttgccagcgccctagcgcccgctcctttcgctttcttcccttcctttctcgc-
cacgttcgccggctttccccgtcaagctctaaatcgggggctccctttagggttc-
cgatttagtgctttacggcacctcgaccccaaaaaacttgattagggtgatg-
gttcacgtagtgggccatcgccccgatagacggtttttcgccctttgacgctg-
gagttcacgttcctcaatagtggactcttgttccaaactggaacaacactcaacc-
ctatctcggtctattcttttgatttataagggatttttccgatttcggcctattggt-
taaaaaatgagctgatttaacaaaaatttaacgcgaattttaacaaaatat-
taacgtttataatttcaggtggcatctttcggggaaatgtgcgcggaacccc-
tatttgtttatttttctaaatacattcaaatatgtatccgctcatgagacaataacc-
ctgataaatgcttcaataatattgaaaaaggaagagtatgagtattcaacatttc-
cgtgtcgcccttattcccttttttgcggcattttgccttcctgtttttgctcaccca-
gaaacgctggtgaaagtaaaagatgctgaagatcagttgggtgcac-
gagtgggttacatcgaactggatctcaatagtggtaagatccttga-
gagttttcgccccgaagaacgttttccaatgatgagcacttttaaagttctgctat-
gtggcgcggtattatcccgtattgacgccgggcaagagcaactcggtcgccg-
catacactattctcagaatgacttggttgagtactcaccagtcacagaaaag-
catcttacggatggcatgacagtaagagaattatgcagtgctgccataaccat-
gagtgataacactgcggccaacttacttctgacaacgatcggaggaccgaag-
gagctaaccgcttttttgcacaacatgggggatcatgtaactcgc-
cttgatcgttgggaaccggagctgaatgaagccataccaaacgacgagcgt-
gacaccacgatgcctgtagtaatggtaacaacgttgcgcaaactattaactg-
gcgaactacttactctagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′
Sequence of AAV2/5 U6 lucRI-4(antisense)
5′-agcgcccaatacgcaaaccgcctctccccgcgcgttggccgattcattaatg
cagctggcacgacaggtttcccgactggaaagcgggcagtgagcgcaacg-
caattaatgtgagttagctcactcattaggcaccccaggctttacactttat-
gcttccggctcgtatgttgtgtggaattgtgagcggataacaatttca-
cacaggaaacagctatgaccatgattacgccagatttaattaaggct-
gcgcgctcgctcgctcactgaggccgcccgggcaaagcccgggcgtcgggc-
gacctttggtcgcccggcctcagtgagcgagcgagcgcgcaga-
gagggagtggccaactccatcactaggggttccttgtagttaatgattaacc-
cgccatgctacttatctacgtagccatgctctaggaagatcggaattcgccct-
taagctagcccccagtggaaagacgcgcaggcaaaacgcaccacgtgacg-
gagcgtgaccgcgcgccgagcccaaggtcgggcaggaagagggcctatttc-
ccatgattccttcatatttgcatatacgatacaaggctgttagagagataatta-
gaattaatttgactgtaaacacaaagatattagtacaaaatacgtgacgta-
gaaagtaataatttcttgggtagtttgcagttttaaaattatgttttaaaatggac-
tatcatatgcttaccgtaacttgaaagtatttcgatttcttggctt-
tatatatcttgtggaaaggacgaaacacctcgaagtactcagcgtaagttttctc-
gagttaagggcgaattcccgattaggatcttcctagagcatggctacgta-
gataagtagcatggcgggttaatcattaactacaaggaacccctagtgatg-
gagttggccactccctctctgcgcgctcgctcgctcactgaggccgggcgac-
caaaggtcgcccgacgcccgggctttgcccgggcggcctcagtgagcgagc-
gagcgcgcagccttaattaacctaattcactggccgtcgttttacaacgtcgt-
gactgggaaaaccctggcgttacccaacttaatcgccttgcagcacatcccc-
ctttcgccagctggcgtaatagcgaagaggcccgcaccgatcgcccttcc-
caacagttgcgcagcctgaatggcgaatgggacgcgccctgtagcggcg-
cattaagcgcggcgggtgtggtggttacgcgcagcgtgaccgcta-
cacttgccagcgccctagcgcccgctcctttcgctttcttcccttcctttctcgc-
cacgttcgccggctttccccgtcaagctctaaatcgggggctccctttagggttc-
cgatttagtgctttacggcacctcgaccccaaaaaacttgattagggtgatg-
gttcacgtagtgggccatcgccccgatagacggtttttcgccctttgacgctg-
gagttcacgttcctcaatagtggactcttgttccaaactggaacaacactcaacc-
ctatctcggtctattcttttgatttataagggatttttccgatttcggcctattggt-
taaaaaatgagctgatttaacaaaaatttaacgcgaattttaacaaaatat-
taacgtttataatttcaggtggcatctttcggggaaatgtgcgcggaacccc-
tatttgtttatttttctaaatacattcaaatatgtatccgctcatgagacaataacc-
ctgataaatgcttcaataatattgaaaaaggaagagtatgagtattcaacatttc-
cgtgtcgcccttattcccttttttgcggcattttgccttcctgtttttgctcaccca-
gaaacgctggtgaaagtaaaagatgctgaagatcagttgggtgcac-
gagtgggttacatcgaactggatctcaatagtggtaagatccttga-
gagttttcgccccgaagaacgttttccaatgatgagcacttttaaagttctgctat-
gtggcgcggtattatcccgtattgacgccgggcaagagcaactcggtcgccg-
catacactattctcagaatgacttggttgagtactcaccagtcacagaaaag-
catcttacggatggcatgacagtaagagaattatgcagtgctgccataaccat-
gagtgataacactgcggccaacttacttctgacaacgatcggaggaccgaag-
gagctaaccgcttttttgcacaacatgggggatcatgtaactcgc-
cttgatcgttgggaaccggagctgaatgaagccataccaaacgacgagcgt-
gacaccacgatgcctgtagtaatggtaacaacgttgcgcaaactattaactg-
gcgaactacttactctagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′
Sequence of AAV2/2 U6 eGFPRI-1a
5′-agcgcccaatacgcaaaccgcctctccccgcgcgttggccgattcattaatg
cagctggcacgacaggtttcccgactggaaagcgggcagtgagcgcaacg-
caattaatgtgagttagctcactcattaggcaccccaggctttacactttat-
gcttccggctcgtatgttgtgtggaattgtgagcggataacaatttca-
cacaggaaacagctatgaccatgattacgccagatttaattaaggct-
gcgcgctcgctcgctcactgaggccgcccgggcaaagcccgggcgtcgggc-
gacctttggtcgcccggcctcagtgagcgagcgagcgcgcaga-
gagggagtggccaactccatcactaggggttccttgtagttaatgattaacc-
cgccatgctacttatctacgtagccatgctctaggaagatcggaattcgccct-
taagctagcccccagtggaaagacgcgcaggcaaaacgcaccacgtgacg-
gagcgtgaccgcgcgccgagcccaaggtcgggcaggaagagggcctatttc-
ccatgattccttcatatttgcatatacgatacaaggctgttagagagataatta-
gaattaatttgactgtaaacacaaagatattagtacaaaatacgtgacgta-
gaaagtaataatttcttgggtagtttgcagttttaaaattatgttttaaaatggac-
tatcatatgcttaccgtaacttgaaagtatttcgatttcttggctt-
tatatatcttgtggaaaggacgaaacaccgaagaagtcgtgctgcttcttcaa-
gagagaagcagcacgacttcttcttttctcgagttaagggcgaattccc-
gattaggatcttcctagagcatggctacgtagataagtagcatggcgggt-
taatcattaactacaaggaacccctagtgatggagttggccactccctctct-
gcgcgctcgctcgctcactgaggccgggcgaccaaaggtcgcccgacgcc-
cgggctttgcccgggcggcctcagtgagcgagcgagcgcgcagccttaat-
taacctaattcactggccgtcgttttacaacgtcgtgactgggaaaaccctg-
gcgttacccaacttaatcgccttgcagcacatccccctttcgccagctggcg-
taatagcgaagaggcccgcaccgatcgcccttcccaacagttgcgcagcct-
gaatggcgaatgggacgcgccctgtagcggcgcattaagcgcggcgggt-
gtggtggttacgcgcagcgtgaccgctacacttgccagcgccctagcgcc-
cgctcctttcgctttcttcccttcctttctcgccacgttcgccggctttccccgt-
caagctctaaatcgggggctccctttagggttccgatttagtgctttacggcac-
ctcgaccccaaaaaacttgattagggtgatggttcacgtagtgggccatcgcc-
ccgatagacggtttttcgccctttgacgctggagttcacgttcctcaatagtg-
gactcttgttccaaactggaacaacactcaaccctatctcggtctattcttttgatt-
tataagggatttttccgatttcggcctattggttaaaaaatgagctgatttaa-
caaaaatttaacgcgaattttaacaaaatattaacgtttataatttcaggtg-
gcatctttcggggaaatgtgcgcggaacccctatttgtttatttttctaaata-
cattcaaatatgtatccgctcatgagacaataaccctgataaat-
gcttcaataatattgaaaaaggaagagtatgagtattcaacatttccgtgtcgcc-
cttattcccttttttgcggcattttgccttcctgtttttgctcacccagaaacgctg-
gtgaaagtaaaagatgctgaagatcagttgggtgcacgagtgggttacatc-
gaactggatctcaatagtggtaagatccttgagagttttcgccccgaa-
gaacgttttccaatgatgagcacttttaaagttctgctatgtggcgcggtattatc-
ccgtattgacgccgggcaagagcaactcggtcgccgcatacactattctca-
gaatgacttggttgagtactcaccagtcacagaaaagcatcttacggatg-
gcatgacagtaagagaattatgcagtgctgccataaccatgagtgataacact-
gcggccaacttacttctgacaacgatcggaggaccgaaggagctaac-
cgcttttttgcacaacatgggggatcatgtaactcgccttgatcgttgggaac-
cggagctgaatgaagccataccaaacgacgagcgtgacaccacgatgcctg-
tagtaatggtaacaacgttgcgcaaactattaactggcgaactacttactc-
tagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′
Sequence of AAV2/5 pol1 lucRI
5′-agcgcccaatacgcaaaccgcctctccccgcgcgttggccgattcattaatg
cagctggcacgacaggtttcccgactggaaagcgggcagtgagcgcaacg-
caattaatgtgagttagctcactcattaggcaccccaggctttacactttat-
gcttccggctcgtatgttgtgtggaattgtgagcggataacaatttca-
cacaggaaacagctatgaccatgattacgccagatttaattaaggct-
gcgcgctcgctcgctcactgaggccgcccgggcaaagcccgggcgtcgggc-
gacctttggtcgcccggcctcagtgagcgagcgagcgcgcaga-
gagggagtggccaactccatcactaggggttccttgtagttaatgattaacc-
cgccatgctacttatctacgtagccatgctctaggaagatcggaattcgccct-
taagctagctttccgagtccccgtggggagccggggaccgtcccgcccc-
cgtcccccgggtgccggggagcggtccctctgccgcgatcctttctggc-
gagtccccgtgcggagtcggagagcgctccctgagcgcgcgtgcggcccga-
gaggtcgcgcctggccggccttcggtccctcgtgtgtcccggtcgtag-
gaggggccggccgaaaatgcttccggctcccgctctggagacacgggccg-
gccccctgcgtgtggcacgggcggccgggagggcgtccccggcccg-
gcgctgctcccgcgtgtgtcctggggttgaccagagggccccgggcgctc-
cgtgtgtggctgcgatggtggcgtttttggggacaggtgtccgt-
gtcgcgcgtcgcctgggccggcggcgtggtcggtgacgcgacctcccggcc-
ccggggaggtatatctttcgctccgagtcggcattttgggccgccgggt-
tattcttacgctgagtacttcgattcaagagatcgaagtactcagcgtaagag-
gtcgaccagattaatccgctcgagttaagggcgaattcccgattaggatcttcc-
tagagcatggctacgtagataagtagcatggcgggttaatcattaactacaag-
gaacccctagtgatggagttggccactccctctctgcgcgctcgctcgctcact-
gaggccgggcgaccaaaggtcgcccgacgcccgggctttgcccgggcggc-
ctcagtgagcgagcgagcgcgcagccttaattaacctaattcactggc-
cgtcgttttacaacgtcgtgactgggaaaaccctggcgttacccaact-
taatcgccttgcagcacatccccctttcgccagctggcgtaatagcgaagag-
gcccgcaccgatcgcccttcccaacagttgcgcagcctgaatggc-
gaatgggacgcgccctgtagcggcgcattaagcgcggcgggtgtggtggt-
tacgcgcagcgtgaccgctacacttgccagcgccctagcgcccgctc-
ctttcgctttcttcccttcctttctcgccacgttcgccggctttccccgtcaagctc-
taaatcgggggctccctttagggttccgatttagtgctttacggcacctcgaccc-
caaaaaacttgattagggtgatggttcacgtagtgggccatcgccccgata-
gacggtttttcgccctttgacgctggagttcacgttcctcaatagtg-
gactcttgttccaaactggaacaacactcaaccctatctcggtctattcttttgatt-
tataagggatttttccgatttcggcctattggttaaaaaatgagctgatttaa-
caaaaatttaacgcgaattttaacaaaatattaacgtttataatttcaggtg-
gcatctttcggggaaatgtgcgcggaacccctatttgtttatttttctaaata-
cattcaaatatgtatccgctcatgagacaataaccctgataaat-
gcttcaataatattgaaaaaggaagagtatgagtattcaacatttccgtgtcgcc-
cttattcccttttttgcggcattttgccttcctgtttttgctcacccagaaacgctg-
gtgaaagtaaaagatgctgaagatcagttgggtgcacgagtgggttacatc-
gaactggatctcaatagtggtaagatccttgagagttttcgccccgaa-
gaacgttttccaatgatgagcacttttaaagttctgctatgtggcgcggtattatc-
ccgtattgacgccgggcaagagcaactcggtcgccgcatacactattctca-
gaatgacttggttgagtactcaccagtcacagaaaagcatcttacggatg-
gcatgacagtaagagaattatgcagtgctgccataaccatgagtgataacact-
gcggccaacttacttctgacaacgatcggaggaccgaaggagctaac-
cgcttttttgcacaacatgggggatcatgtaactcgccttgatcgttgggaac-
cggagctgaatgaagccataccaaacgacgagcgtgacaccacgatgcctg-
tagtaatggtaacaacgttgcgcaaactattaactggcgaactacttactc-
tagcttcccggcaacaattaatagactggatggaggcg-
gataaagttgcaggaccacttctgcgctcggcccttccggctggctggtt-
tattgctgataaatctggagccggtgagcgtgggtctcgcggtat-
cattgcagcactggggccagatggtaagccctcccgtatcgtagttatctacac-
gacggggagtcaggcaactatggatgaacgaaatagacagatcgctga-
gataggtgcctcactgattaagcattggtaactgtcagaccaagtttact-
catatatactttagattgatttaaaacttcatttttaatttaaaaggatctaggt-
gaagatcctttttgataatctcatgaccaaaatcccttaacgtgagttttcgttc-
cactgagcgtcagaccccgtagaaaagatcaaaggatcttcttgagatc-
ctttttttctgcgcgtaatctgctgcttgcaaacaaaaaaaccaccgc-
taccagcggtggtttgtttgccggatcaagagctaccaactctttttccgaag-
gtaactggcttcagcagagcgcagataccaaatactgtccttctagtgtagccg-
tagttaggccaccacttcaagaactctgtagcaccgcctacatacctcgctctgc-
taatcctgttaccagtggctgctgccagtggcgataagtcgtgtcttac-
cgggttggactcaagacgatagttaccggataaggcgcagcggtcgggct-
gaacggggggttcgtgcacacagcccagcttggagcgaacgacctacacc-
gaactgagatacctacagcgtgagctatgagaaagcgccacgcttccc-
gaagggagaaaggcggacaggtatccggtaagcggcagggtcg-
gaacaggagagcgcacgagggagcttccagggggaaacgcctggtatctt-
tatagtcctgtcgggtttcgccacctctgacttgagcgtcgatttttgtgat-
gctcgtcaggggggcggagcctatggaaaaacgccagcaacgcggcctttt-
tacggttcctggccttttgctgcggttttgctcacatgttctttcctgcgttatccc-
ctgattctgtggataaccgtattaccgcctttgagtgagctgataccgctcgc-
cgcagccgaacgaccgagcgcagcgagtcagtgagcgaggaagcggaag-3′

Prior Art

U.S. Patent Documents

U.S. Patent Documents

| | | |
|---|---|---|
| U.S. Pat. No. 6,506,559 | Fire et al. | January, 2003 |
| U.S. Pat. No. 6,573,099 | Graham | June, 2003 |
| U.S. Pat. No. 5,658,785 | Johnson | August, 1997 |
| U.S. Pat. No. 5,107,065 | Shewmaker et al. | April, 1992 |
| U.S. Pat. No. 5,139,941 | Muzyczka et al. | August, 1992 |
| U.S. Pat. No. 6,458,587 | Ferrari et al. | October, 2002 |
| U.S. Pat. No. 5,478,745 | Samulski et al. | December, 1995 |

References
1. Grassi, G. and J. C. Marini, Ribozymes: structure, function, and potential therapy for dominant genetic disorders. Ann Med, 1996. 28(6): p. 499-510.
2. Lonnberg, H. and E. Vuorio, Towards genomic drug therapy with antisense oligonucleotides. Ann Med, 1996. 28(6): p. 511-22.
3. Beerli, R. R. and C. F. Barbas, 3rd, Engineering polydactyl zinc-finger transcription factors. Nat Biotechnol, 2002. 20(2): p. 135-41.
4. Imanishi, M., et al., Design of novel zinc finger proteins: towards artificial control of specific gene expression. Eur J Pharm Sci, 2001. 13(1): p. 91-7.
5. Cohen, P. A., Intrabodies. Targeting scFv expression to eukaryotic intracellular compartments. Methods Mol Biol, 2002. 178: p. 367-78.
6. McManus, M. T. and P. A. Sharp, Gene silencing in mammals by small interfering RNAs. Nat Rev Genet, 2002. 3(10): p. 737-47.
7. Sharp, P. A., RNA interference—2001. Genes Dev, 2001. 15(5): p. 485-90.
8. Sharp, P. A. and P. D. Zamore, Molecular biology. RNA interference. Science, 2000. 287(5462): p. 2431-3.
9. Tuschl, T., et al., Targeted mRNA degradation by double-stranded RNA in vitro. Genes Dev, 1999. 13(24): p. 3191-7.
10. Zamore, P. D., et al., RNAi: double-stranded RNA directs the ATP-dependent cleavage of mRNA at 21 to 23 nucleotide intervals. Cell, 2000. 101(1): p. 25-33.
11. Dejneka, N. S. and J. Bennett, Gene therapy and retinitis pigmentosa: advances and future challenges. Bioessays, 2001. 23(7): p. 662-8.
12. Murata, T., et al., Ocular gene therapy: experimental studies and clinical possibilities. Ophthalmic Res, 1997. 29(5): p. 242-51.
13. Bennett, J., Gene therapy for retinitis pigmentosa. Curr Opin Mol Ther, 2000. 2(4): p. 420-5.
14. Borkhardt, A., Blocking oncogenes in malignant cells by RNA interference—new hope for a highly specific cancer treatment? Cancer Cell, 2002. 2(3): p. 167-8.
15. Wilda, M., et al., Killing of leukemic cells with a BCR/ABL fusion gene by RNA interference (RNAi). Oncogene, 2002. 21(37): p. 5716-24.
16. Brummelkamp, T. R., R. Bernards, and R. Agami, Stable suppression of tumorigenicity by virus-mediated RNA interference. Cancer Cell, 2002. 2(3): p. 243-7.
17. Kitabwalla, M. and R. M. Ruprecht, RNA interference—a new weapon against HIV and beyond. N Engl J Med, 2002. 347(17): p. 1364-7.
18. Surabhi, R. M. and R. B. Gaynor, RNA interference directed against viral and cellular targets inhibits human immunodeficiency Virus Type 1 replication. J Virol, 2002. 76(24): p. 12963-73.
19. Randall, G., A. Grakoui, and C. M. Rice, Clearance of replicating hepatitis C virus replicon RNAs in cell culture by small interfering RNAs. Proc Natl Acad Sci USA, 2003. 100(1): p. 235-40.
20. Caplen, N. J., et al., Rescue of polyglutamine-mediated cytotoxicity by double-stranded RNA-mediated RNA interference. Hum Mol Genet, 2002. 11(2): p. 175-84.
21. Fire, A., et al., Potent and specific genetic interference by double-stranded RNA in Caenorhabditis elegans. Nature, 1998. 391(6669): p. 806-11.
22. Bosher, J. M. and M. Labouesse, RNA interference: genetic wand and genetic watchdog. Nat Cell Biol, 2000. 2(2): p. E31-6.
23. Hammond, S. M., A. A. Caudy, and G. J. Hannon, Post-transcriptional gene silencing by double-stranded RNA. Nat Rev Genet, 2001. 2(2): p. 110-9.
24. Zamore, P. D., RNA interference: listening to the sound of silence. Nat Struct Biol, 2001. 8(9): p. 746-50.
25. Moss, E. G., RNA interference: it's a small RNA world. Curr Biol, 2001. 11(19): p. R772-5.
26. Fjose, A., et al., RNA interference: mechanisms and applications. Biotechnol Annu Rev, 2001. 7: p. 31-57.
27. Tuschl, T., RNA interference and small interfering RNAs. Chembiochem, 2001. 2(4): p. 239-45.
28. Cullen, B. R., RNA interference: antiviral defense and genetic tool. Nat Immunol, 2002. 3(7): p. 597-9.
29. Hannon, G. J., RNA interference. Nature, 2002. 418(6894): p. 244-51.
30. Bernstein, E., et al., Role for a bidentate ribonuclease in the initiation step of RNA interference. Nature, 2001. 409(6818): p. 363-6.
31. Elbashir, S. M., W. Lendeckel, and T. Tuschl, RNA interference is mediated by 21- and 22-nucleotide RNAs. Genes Dev, 2001.15(2): p. 188-200.
32. Elbashir, S. M., et al., Duplexes of 21-nucleotide RNAs mediate RNA interference in cultured mammalian cells. Nature, 2001. 411(6836): p. 494-8.
33. Hutvagner, G., et al., A cellular function for the RNA-interference enzyme Dicer in the maturation of the let-7 small temporal RNA. Science, 2001. 293(5531): p. 834-8.
34. Grishok, A. and C. C. Mello, RNAi (Nematodes: Caenorhabditis elegans). Adv Genet, 2002. 46: p. 339-60.
35. Grishok, A., et al., Genes and mechanisms related to RNA interference regulate expression of the small temporal RNAs that control C. elegans developmental timing. Cell, 2001. 106(1): p. 23-34.
36. Ambros, V., microRNAs: tiny regulators with great potental. Cell, 2001. 107(7): p. 823-6.
37. Grosshans, H. and F. J. Slack, Micro-RNAs: small is plentiful. J Cell Biol, 2002. 156(1): p. 17-21.
38. Moss, E. G., MicroRNAs: hidden in the genome. Curr Biol, 2002. 12(4): p. R138-40.
39. Xia, H., et al ., siRNA-mediated gene silencing in vitro and in vivo. Nat Biotechnol, 2002. 20(10): p. 1006-10.
40. Paddison, P. J., et al., Short hairpin RNAs (shRNAs) induce sequence-specific silencing in mammalian cells. Genes Dev, 2002. 16(8): p. 948-58.
41. Paddison, P. J., A. A. Caudy, and G. J. Hannon, Stable suppression of gene expression by RNAi in mammalian cells. Proc Natl Acad Sci USA, 2002. 99(3): p. 1443-8.
42. Paul, C. P., et al., Effective expression of small interfering RNA in human cells. Nat Biotechnol, 2002. 20(5): p. 505-8.
43. Yu, J. Y., S. L. DeRuiter, and D. L. Turner, RNA interference by expression of short-interfering RNAs and hairpin RNAs in mammalian cells. Proc Natl Acad Sci USA, 2002. 99(9): p. 6047-52.
44. Lee, N. S., et al., Expression of small interfering RNAs targeted against HIV-1 rev transcripts in human cells. Nat Biotechnol, 2002. 20(5): p. 500-5.
45. McCaffrey, A. P., et al., RNA interference in adult mice. Nature, 2002. 418(6893): p. 38-9.
46. Lewis, D. L., et al., Efficient delivery of siRNA for inhibition of gene expression in postnatal mice. Nat Genet, 2002. 32(1): p. 107-8.
47. Sijen, T., et al., On the role of RNA amplification in dsRNA-triggered gene silencing. Cell, 2001. 107(4): p. 465-76.
48. Voorhoeve, P. M. and R. Agami, Knockdown stands up. Trends Biotechnol, 2003. 21(1): p. 2-4.
49. Scarpa, M., et al., Characterization of recombinant helper retroviruses from Moloney-based vectors in ecotropic and amphotropic packaging cell lines. Virology, 1991. 180(2): p. 849-52.
50. Miller, A. D. and G. J. Rosman, Improved retroviral vectors for gene transfer and expression. Biotechniques, 1989. 7(9): p. 980-2, 984-6, 989-90.
51. Miller, A. D., Retrovirus packaging cells. Hum Gene Ther, 1990. 1(1): p. 5-14.
52. Burns, J. C., et al., Vesicular stomatitis virus G glycoprotein pseudotyped retroviral vectors: concentration to very high titer and efficient gene transfer into mammalian and nonmammalian cells. Proc Natl Acad Sci USA, 1993. 90(17): p. 8033-7.
53. Boris-Lawrie, K. A. and H. M. Temin, Recent advances in retrovirus vector technology. Curr Opin Genet Dev, 1993. 3(1): p. 102-9.
54. Li, Z., et al., Murine leukemia induced by retroviral gene marking. Science, 2002. 296(5567): p. 497.
55. Hacein-Bey-Abina, S., et al., A serious adverse event after successful gene therapy for X-linked severe combined immunodeficiency. N Engl J Med, 2003. 348(3): p. 255-6.
56. Haj-Ahmad, Y. and F. L. Graham, Development of a helper-independent human adenovirus vector and its use in the transfer of the herpes simplex virus thymidine kinase gene. J Virol, 1986. (1): p. 267-74.
57. Mittereder, N., et al., Evaluation of the efficacy and safety of in vitro, adenovirus-mediated transfer of the human cystic fibrosis transmembrane conductance regulator cDNA. Hum Gene Ther, 1994. 5(6): p. 717-29.
58. Barr, E., et al., Efficient catheter-mediated gene transfer into the heart using replication-defective adenovirus. Gene Ther, 1994. 1(1): p. 51-8.
59. Bett, A. J., L. Prevec, and F. L. Graham, Packaging capacity and stability of human adenovirus type 5 vectors. J Virol, 1993. 67(10): p. 5911-21.
60. Berkner, K. L., Development of adenovirus vectors for the expression of heterologous genes. Biotechniques, 1988. 6(7): p. 616-29.
61. Seth, P., Adenovirus-dependent release of choline from plasma membrane vesicles at an acidic pH is mediated by the penton base protein. J Virol, 1994. 68(2): p. 1204-6.
62. Rich, D. P., et al., Development and analysis of recombinant adenoviruses for gene therapy of cystic fibrosis. Hum Gene Ther, 1993. 4(4): p. 461-76.
63. Lai, C. M., Y. K. Lai, and P. E. Rakoczy, Adenovirus and adeno-associated virus vectors. DNA Cell Biol, 2002. 21(12): p. 895-913.
64. Owens, R. A., Second generation adeno-associated virus type 2-based gene therapy systems with the potential for preferential integration into AAVS1. Curr Gene Ther, 2002. 2(2): p. 145-59.
65. Paterna, J. C. and H. Bueler, Recombinant adeno-associated virus vector design and gene expression in the mammalian brain. Methods, 2002. 28(2): p. 208-18.
66. Nicklin, S. A. and A. H. Baker, Tropism-modified adenoviral and adeno-associated viral vectors for gene therapy. Curr Gene Ther, 2002. 2(3): p. 273-93.
67. Izraeli, S. and G. Rechavi, Molecular medicine—an overview. Isr Med Assoc J, 2002. 4(8): p. 638-40.
68. Mohr, L. and M. Geissler, [Gene therapy: new developments]. Schweiz Rundsch Med Prax, 2002. 91(51-52): p. 2227-35.
69. Graham, F. L. and A. J. van der Eb, A new technique for the assay of infectivity of human adenovirus 5 DNA. Virology, 1973. 52(2): p. 456-67.
70. Graham, F. L. and A. J. van der Eb, Transformation of rat cells by DNA of human adenovirus 5. Virology, 1973. 54(2): p. 536-9.
71. Chu, G. and P. A. Sharp, SV40 DNA transfection of cells in suspension: analysis of efficiency of transcription and translation of T-antigen. Gene, 1981. 13(2): p. 197-202.
72. Wang, L., et al., Sustained correction of bleeding disorder in hemophilia B mice by gene therapy. Proc Natl Acad Sci USA, 1999. 96(7): p. 3906-10.
73. Brummelkamp, T. R., R. Bernards, and R. Agami, A system for stable expression of short interfering RNAs in mammalian cells. Science, 2002. 296(5567): p. 550-3.
74. Boshart, M., et al., A very strong enhancer is located up-stream of an immediate early gene of human cytomegalovirus. Cell, 1985. 41(2): p. 521-30.
75. No, D., T. P. Yao, and R. M. Evans, Ecdysone-inducible gene expression in mammalian cells and transgenic mice. Proc Natl Acad Sci USA, 1996. 93(8): p. 3346-51.
76. Gossen, M. and H. Bujard, Tight control of gene expression in mammalian cells by tetracycline-responsive promoters. Proc Natl Acad Sci USA, 1992. 89(12): p. 5547-51.
77. Gossen, M., et al., Transcriptional activation by tetracyclines in mammalian cells. Science, 1995. 268(5218): p. 1766-9.
78. Harvey, D. M. and C. T. Caskey, Inducible control of gene expression: prospects for gene therapy. Curr Opin Chem Biol, 1998. 2(4): p. 512-8.
79. Wang, Y., et al., Ligand-inducible and liver-specific target gene expression in transgenic mice. Nat Biotechnol, 1997. (3): p. 239-43.
80. Wang, Y., et al., Positive and negative regulation of gene expression in eukaryotic cells with an inducible transcriptional regulator. Gene Ther, 1997. 4(5): p. 432-41.
81. Magari, S. R., et al., Pharmacologic control of a humanized gene therapy system implanted into nude mice. J Clin Invest, 1997. 100(11): p. 2865-72.
82. Roux, K. H., Using mismatched primer-template pairs in touchdown PCR. Biotechniques, 1994. 16(5): p. 812-4.
83. Hildinger, M., et al., Hybrid vectors based on adeno-as-sociated virus serotypes 2 and 5 for muscle-directed gene transfer. J Virol, 2001. 75(13): p. 6199-203.
84. Kotin, R. M., Prospects for the use of adeno-associated virus as a vector for human gene therapy. Hum Gene Ther, 1994. 5(7): p. 793-801.
85. Capecchi, M. R., High efficiency transformation by direct microinjection of DNA into cultured mammalian cells. Cell, 1980. 22(2 Pt 2): p. 479-88.
86. Shigekawa, K. and W. J. Dower, Electroporation of eukaryotes and prokaryotes: a general approach to the introduction of macromolecules into cells. Biotechniques, 1988. 6(8): p. 742-51.
87. Mannino, R. J. and S. Gould-Fogerite, Liposome mediated gene transfer. Biotechniques, 1988. 6(7): p. 682-90.
88. Felgner, P. L., et al., Lipofection: a highly efficient, lipid-mediated DNA-transfection procedure. Proc Natl Acad Sci USA, 1987. 84(21): p. 7413-7.
89. Graham, F. L., et al., Characteristics of a human cell line transformed by DNA from human adenovirus type 5. J Gen Virol, 1977. 36(1): p. 59-74.
90. Aiello, L., et al., Adenovirus 5 DNA sequences present and RNA sequences transcribed in transformed human embryo kidney cells (HEK-Ad-5 or 293). Virology, 1979. 94(2): p. 460-9.
91. Muzyczka, N., Use of adeno-associated virus as a general transduction vector for mammalian cells. Curr Top Microbiol Immunol, 1992. 158: p. 97-129.
92. Thomson, B. J., et al., Human herpesvirus 6 (HHV-6) is a helper virus for adeno-associated virus type 2 (AAV-2) and the AAV-2 rep gene homologue in HHV-6 can mediate AAV-2 DNA replication and regulate gene expression. Virology, 1994. 204(1): p. 304-11.
93. Samulski, R. J., L. S. Chang, and T. Shenk, Helper-free stocks of recombinant adeno-associated viruses: normal integration does not require viral gene expression. J Virol, 1989. 63(9): p. 3822-8.
94. McCarty, D. M., M. Christensen, and N. Muzyczka, Sequences required for coordinate induction of adeno-associated virus p19 and p40 promoters by Rep protein. J Virol, 1991. 65(6): p. 2936-45.
95. Schlehofer, J. R., M. Ehrbar, and H. zur Hausen, Vaccinia virus, herpes simplex virus, and carcinogens induce DNA amplification in a human cell line and support replication of a helpervirus dependent parvovirus. Virology, 1986. 152(1): p. 110-7.
96. McPherson, R. A., L. J. Rosenthal, and J. A. Rose, Human cytomegalovirus completely helps adeno-associated virus replication. Virology, 1985. 147(1): p. 217-22.
97. Buller, R. M., et al., Herpes simplex virus types 1 and 2 completely help adenovirus-associated virus replication. J Virol, 1981. 40(1): p. 241-7.
98. Janik, J. E., M. M. Huston, and J. A. Rose, Locations of adenovirus genes required for the replication of adenovirus-associated virus. Proc Natl Acad Sci USA, 1981. 78(3): p. 1925-9.
99. Young, J. F. and H. D. Mayor, Adeno-associated virus—an extreme state of viral defectiveness. Prog Med Virol, 1979. 25: p. 113-32.
100. Vickers, T. A., et al., Efficient reduction of target RNA's by siRNA and RNase H dependent antisense agents: A comparative analysis. J Biol Chem, 2002.
101. Sui, G., et al., A DNA vector-based RNAi technology to suppress gene expression in mammalian cells. Proc Natl Acad Sci USA, 2002. 99(8): p. 5515-20.
102. Miyagishi, M. and K. Taira, U6 promoter-driven siRNAs with four uridine 3′ overhangs efficiently suppress targeted gene expression in mammalian cells. Nat Biotechnol, 2002. 20(5): p. 497-500.
103. Elbashir, S. M., et al., Functional anatomy of siRNAs for mediating efficient RNAi in Drosophila melanogaster embryo lysate. Embo J, 2001. 20(23): p. 6877-88.
104. Auricchio, A., et al., Isolation of highly infectious and pure adeno-associated virus type 2 vectors with a single-step gravity-flow column. Hum Gene Ther, 2001. 12(1): p. 71-6.
105. Auricchio, A., et al., A single-step affinity column for purification of serotype-5 based adeno-associated viral vectors. Mol Ther, 2001. 4(4): p. 372-4.

1. A method of decreasing the expression of a target gene in a cell of a mammalian subject comprising administering to the subject in vivo a therapeutically effective amount of an RNAi expression cassette, comprising:
(a) providing a recombinant adeno-associated viral vector, wherein said vector comprises said RNAi expression cassette whose RNA expression product(s) directly or indirectly lead to the decrease of expression of an RNAi target gene, wherein the RNA expression product(s) of the RNAi expression cassette comprise a nucleotide sequence that hybridizes under stringent conditions to a nucleotide sequence of the RNAi target gene mRNA transcript
(b) delivering said recombinant adeno-associated viral vector to and/or within said mammalian subject wherein transduction of suitable target cells results in expression of said RNAi expression cassette.

2. A method of decreasing the expression of (at least) one target gene in a cell of a mammalian subject comprising administering to the subject in vivo a therapeutically effective amount of (at least) one RNAi expression cassette, comprising:
(a) providing (at least) one recombinant adeno-associated viral vector, wherein said vector comprises (at least) one RNAi expression cassette whose RNA expression product(s) directly or indirectly lead to the decrease of expression of an RNAi target gene, wherein the RNA expression product(s) of the RNAi expression cassette comprise a nucleotide sequence that hybridizes under stringent conditions to a nucleotide sequence of the RNAi target gene mRNA transcript
(b) delivering said recombinant adeno-associated viral vector(s) to and/or within said mammalian subject wherein transduction of suitable target cells results in expression of said RNAi expression cassette.

3. The method of claims 1 and 2, wherein expression of the RNA coding region of the RNAi expression cassette results in the down-regulation of the expression of the RNAi target gene, wherein the target gene comprises a sequence that is at least about 90% identical with the RNA coding region.

4. The method of claims 1 and 2, in which the RNAi target gene expression is inhibited by at least 10%.

5. The method of claims 1 and 2, wherein said RNAi expression cassette(s) encode one or more RNA molecules which are capable of forming an RNA interference inducing double-stranded RNA complex.

6. The method of claims 1 and 2, wherein said RNAi expression cassette encodes (at least) one RNA molecule which is self-complementary.

7. The method of claims 1 and 2, wherein said RNAi expression cassette encodes (at least) two separate complementary single-stranded RNA molecules.

8. The RNA molecule or RNA molecules of claims 6 and 7, wherein said RNA molecule or RNA molecules are capable of forming an RNA interference inducing double-stranded RNA complex.

9. The method of claims 1 and 2, wherein (at least) two recombinant adeno-associated viral vectors are used with each vector comprising its own RNAi expression cassette, and each RNAi expression cassette encoding at least one RNA molecule which is complementary to the RNA molecule expressed by the other RNAi expression cassette.

10. The RNA molecule(s) of claims 5, 6, 7, 8 and 9 having a nucleotide sequence which is substantially identical and/or complementary to at least a part of the RNAi target gene.

11. The RNA molecule(s) of claims 5, 6, 7, 8 and 9 with the RNA molecule(s) being siRNA.

12. The method of claims 1 and 2, wherein said RNAi expression cassette encodes a self-complementary RNA molecule comprising a sense region, a loop region and an antisense region.

13. The method of claim 12, wherein the loop region is about 2 to about 10 nucleotides in length.

14. The method of claim 12, wherein the sense region and the antisense region are each between about 10 and about 30 nucleotides in length.

15. The method of claim 12, wherein the sense region hybridizes under stringent conditions to a nucleotide sequence of the RNAi target gene, and the antisense region, which is a complementary inverted repeat of said sense region, hybridizes to said sense region to form a hairpin structure.

16. The method of claims 1 and 2, wherein said RNAi expression cassette comprises a first promoter and a second promoter, each operably linked to an RNA coding region, such that expression of the RNA coding region from the first promoter results in the synthesis of a first RNA molecule and expression of the RNA coding region from the second promoter results in the synthesis of a second RNA molecule substantially complementary to the first RNA molecule.

17. The method of claims 1 and 2, wherein said RNAi expression cassette comprises two promoters operably linked to the same RNA coding region, such that expression of the RNA coding region from the first promoter results in the synthesis of a first RNA molecule and expression of the RNA coding region from the second promoter results in the synthesis of a second RNA molecule substantially complementary to the first RNA molecule.

18. The method of claims 1 and 2, wherein said RNAi expression cassette encodes (at least) two RNA molecules, wherein
(a) one of the (at least) two RNA molecules consists essentially of a ribonucleotide sequence which corresponds to a nucleotide sequence of the RNAi target gene and another of the (at least) two RNA molecules consists essentially of a ribonucleotide sequence which is complementary to said nucleotide sequence of the RNAi target gene
(b) the (at least) two RNA molecules are separate complementary strands that hybridize to each other to form a double-stranded RNA complex, and the double-stranded RNA complex directly or indirectly inhibits expression of the RNAi target gene.

19. The method of claims 1 and 2, wherein said RNAi expression cassette comprises a promoter operably linked to a DNA sequence which, when expressed by a host cell produces one RNA molecule having:
(a) homology to at least one target mRNA expressed by the host cell
(b) two (internally) complementary RNA regions
wherein the expressed RNA reduces the intracellular concentration of the target mRNA or any substantially similar endogenous mRNA either directly or indirectly.

20. The method of claims 1 and 2, wherein said RNAi expression cassette encodes (at least) one RNA molecule for inhibiting expression of a target gene, comprising a first nucleotide sequence that hybridizes under stringent conditions to a nucleotide sequence of the RNAi target gene, and a second nucleotide sequence which is a complementary inverted repeat of said first nucleotide sequence and hybridizes to said first nucleotide sequence to form a hairpin structure.

21. The RNA molecule of claim 19, wherein the two nucleotide sequences are joined by an RNA loop structure.

22. The method of claims 1 and 2, wherein expression of said RNAi expression cassette leads to the generation of a double-stranded RNA complex comprising:
(a) a first RNA portion capable of hybridizing under physiological conditions to at least a part of an mRNA molecule encoded by a gene; and
(b) a second RNA portion wherein at least a part of the second RNA portion is capable of hybridizing under physiological conditions to the first RNA portion.

23. The RNA complex of claim 22 wherein the first and second portions are separate ribonucleic acid molecules.

24. The RNA complex of claim 22 wherein the first and second portions are comprised within the same RNA molecule.

25. The method of claims 1 and 2, wherein said RNAi expression cassette encodes a linear RNA molecule capable of forming a double-stranded RNA complex wherein the RNA molecule comprises:
(a) a first portion that hybridizes under physiologic conditions to at least a portion of an mRNA molecule encoded by a gene; and
(b) a second portion wherein at least part of the second portion is capable of hybridizing to the first portion to form a hairpin double-stranded RNA complex.

26. The linear RNA molecule of claim 25 further comprising a third portion of ribonucleic acid interposed between the first and second portions.

27. The linear RNA molecule of claim 26 wherein the third portion promotes hybridization between the first and second portion.

28. The method of claims 1 and 2, wherein said RNAi expression cassette encodes a linear RNA molecule capable of forming a double-stranded RNA complex wherein the RNA molecule comprises:
(a) a first portion that comprises a region of RNA that is complementary to at least a portion of an mRNA molecule encoded by a gene
(b) a second portion capable of hybridizing to at least part of the first portion
(c) a third portion positioned between the first and second portions to facilitate the hybridization of the first and second portions with one another.

29. The linear RNA molecule of claim 22 and 25 wherein the second sequence comprises a transcription termination signal positioned at the 3′ end of the linear RNA molecule.

30. The method of claims 1 and 2, wherein the recombinant adeno-associated viral vector further comprises a gene of interest.

31. The method of claims 1 and 2, wherein the rAAV vector is of serotype 1, 2, 3, 4, 5, 6, 7 or 8 or any homologous serotypes or hybrids thereof.

32. The method of claims 1 and 2, wherein said RNAi expression cassette comprises an RNA Polymerase III promoter.

33. The method of claims 1 and 2, wherein said RNAi expression cassette comprises an RNA Polymerase II promoter.

34. The method of claims 1 and 2, wherein said RNAi expression cassette comprises an RNA Polymerase I promoter.

35. The method of claims 1 and 2, wherein said RNAi target gene causes or is likely to cause disease.

36. The method of claims 1 and 2, wherein said RNAi target genes are the Rhodopsin gene, the CCR5 gene, the CXCR4 gene, the VEGF gene, the HIF gene or any other gene of therapeutic interest.

37. The method of claims 1 and 2, wherein said RNAi target gene is the Rhodopsin gene.

38. The method of claims 1 and 2, wherein said transduced cells are cells of and/or in the eye, retinal cells, retinal pigment epithelial cells, photoreceptor cells, cells of the eye, gut cells, muscle cells, lung cells, intestinal cells, liver cells, pancreatic cells, hematopoietic cells, stem cells, skin cells, endothelial cells, neurons, cells of ectodermal origin, cells of neurodermal original, cells of endodermal original and/or brain cells.

39. The method of claims 1 and 2, wherein said transduced cells are photoreceptor cells.

40. The pharmaceutical preparation comprising a recombinant adeno-associated viral vector comprising an RNAi expression cassette as claimed in claim 1.

41. The pharmaceutical preparation as claimed in claim 40, wherein said preparation is suitable for and/or administered by intravenous administration.

42. The pharmaceutical preparation as claimed in claim 40, wherein said preparation is suitable for and/or administered by intra-arterial administration.

43. The pharmaceutical preparation as claimed in claim 40, wherein said preparation is suitable for and/or administered by intracavity injection.

44. The pharmaceutical preparation as claimed in claim 40, wherein said preparation is suitable for and/or administered by injection into tissue.

45. The pharmaceutical preparation as claimed in claim 40, wherein said preparation is suitable for and/or administered by injection into gaps in tissue.

46. The pharmaceutical preparation as claimed in claim 40, wherein said preparation is suitable for and/or administered by local administration.

47. The pharmaceutical preparation as claimed in claim 40, wherein said preparation is suitable for and/or administered by inhalation and/or nasal instillation.

48. The pharmaceutical preparation as claimed in claim 40, wherein said preparation is suitable for and/or administered by intraocular and/or intravitreal administration.

49. A method for treating a mammalian subject with an autosomal-dominant disorder or other disease including cancer and infectious diseases by administering to the subject an adeno-associated viral vector for initiating decrease of RNAi target gene expression at the mRNA level, wherein the method comprises using RNAi to achieve post-transcriptional gene silencing.

50. The method of claim 49, wherein the mammalian subject is a human patient.


SEQUENCE LISTING (11 sequences)

SEQ ID NO: 1 - DNA, 6437 bp, Artificial
  sequence for recombinant adeno-associated viral vector, including plasmid backbone, with AAV2 internal terminal repeats that flank expression cassette; referred to as AAV2/2 CMV luciferase
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - protein, 550 aa, Artificial
  sequence for recombinant adeno-associated viral vector, including plasmid backbone, with AAV2 internal terminal repeats that flank expression cassette; referred to as AAV2/2 CMV luciferase
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 3 - DNA, 6437 bp, Artificial; identical to SEQ ID NO: 1
  sequence for recombinant adeno-associated viral vector, including plasmid backbone, with AAV2 internal terminal repeats that flank expression cassette; referred to as AAV2/5 CMV luciferase

SEQ ID NO: 4 - protein, 550 aa, Artificial; identical to SEQ ID NO: 2
  sequence for recombinant adeno-associated viral vector, including plasmid backbone, with AAV2 internal terminal repeats that flank expression cassette; referred to as AAV2/5 CMV luciferase

SEQ ID NO: 5 - DNA, 3618 bp, Artificial
  sequence for recombinant adeno-associated viral vector, including plasmid backbone, with AAV2 internal terminal repeats that flank expression cassette; referred to as AAV2/5 U6 lucRI-1a
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcccccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccttacgctgagtacttcgattcaagagatcgaagtactcagcgtaagtttttctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 6 - DNA, 3920 bp, Artificial
  sequence for recombinant adeno-associated viral vector, including plasmid backbone, with AAV2 internal terminal repeats that flank expression cassette; referred to as AAV2/5 U6 lucRI-1b
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcttacgctgagtacttcgattcaagagatcgaagtactcagcgtaaggctagcacacaaaaaaccaacacacagatctaatgaaaataaagatcttttactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 7 - DNA, 3923 bp, Artificial
  sequence for recombinant adeno-associated viral vector, including plasmid backbone, with AAV2 internal terminal repeats that flank expression cassette; referred to as AAV2/5 U6/U6 lucRIU6-3
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcccccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctttttcttacgctgagtacttcgatttttggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttgggctcggcgcgcggtcacgctccgtcacgtggtgcgttttgcctgcgcgtctttccactggggctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 8 - DNA, 3589 bp, Artificial
  sequence for recombinant adeno-associated viral vector, including plasmid backbone, with AAV2 internal terminal repeats that flank expression cassette; referred to as AAV2/5 U6 lucRI-4(sense)
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcccccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccttacgctgagtacttcgattttctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 9 - DNA, 3589 bp, Artificial
  sequence for recombinant adeno-associated viral vector, including plasmid backbone, with AAV2 internal terminal repeats that flank expression cassette; referred to as AAV2/5 U6 lucRI-4(antisense)
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcccccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctcgaagtactcagcgtaagttttctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 10 - DNA, 3617 bp, Artificial
  sequence for recombinant adeno-associated viral vector, including plasmid backbone, with AAV2 internal terminal repeats that flank expression cassette; referred to as AAV2/2 U6 eGFPRI-1a
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcccccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaagaagtcgtgctgcttcttcaagagagaagcagcacgacttcttcttttctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 11 - DNA, 3787 bp, Artificial
  sequence for recombinant adeno-associated viral vector, including plasmid backbone, with AAV2 internal terminal repeats that flank expression cassette; referred to as AAV2/5 pol1 lucRI
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctttccgagtccccgtggggagccggggaccgtcccgcccccgtcccccgggtgccggggagcggtccctctgccgcgatcctttctggcgagtccccgtgcggagtcggagagcgctccctgagcgcgcgtgcggcccgagaggtcgcgcctggccggccttcggtccctcgtgtgtcccggtcgtaggaggggccggccgaaaatgcttccggctcccgctctggagacacgggccggccccctgcgtgtggcacgggcggccgggagggcgtccccggcccggcgctgctcccgcgtgtgtcctggggttgaccagagggccccgggcgctccgtgtgtggctgcgatggtggcgtttttggggacaggtgtccgtgtcgcgcgtcgcctgggccggcggcgtggtcggtgacgcgacctcccggccccggggaggtatatctttcgctccgagtcggcattttgggccgccgggttattcttacgctgagtacttcgattcaagagatcgaagtactcagcgtaagaggtcgaccagattaatccgctcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag